(19) United States
(12) Patent Application Publication
(10) Pub. No.: US 2017/0050961 A1
(43) Pub. Date: Feb. 23, 2017

(54) IMIDAZO[1,2-A]PYRIDINES AS SOLUBLE GUANYLATE CYCLASE STIMULATORS FOR THE TREATMENT OF CARDIOVASCULAR DISEASES

(71) Applicant: BAYER PHARMA AKTIENGESELLSCHAFT, Berlin (DE)

(72) Inventors: Alexandros VAKALOPOULOS, Hilden (DE); Gaelle VALOT, Wuppertal (DE); Markus FOLLMANN, Köln (DE); Frank WUNDER, Wuppertal (DE); Johannes-Peter STASCH, Grottaferrata (RM) (IT); Tobias MARQUARDT, Wuppertal (DE); Lisa DIETZ, Wuppertal (DE); Volkhart Min-Jian LI, Velbert (DE); Nicholas Charles RAY, Harlow Essex (GB); Dominika BACHERA, Groningen (NL)

(73) Assignee: BAYER PHARMA AKTIENGESELLSCHAFT, Berlin (DE)

(21) Appl. No.: 15/308,148

(22) PCT Filed: Apr. 29, 2015
(86) PCT No.: PCT/EP2015/059282

§ 371 Date: Nov. 1, 2016

(30) Foreign Application Priority Data
May 2, 2014 (EP) 14166909.3

(51) Int. Cl. C07D 471/04; C07F 7/08; A61K 31/437; A61K 31/695; A61K 45/06; A61K 31/444

(52) U.S. Cl. CPC C07D 471/04; A61K 45/06; A61K 31/444; A61K 31/437; A61K 31/695; C07F 7/083; C07F 7/0812

(57) ABSTRACT

The present application relates to novel substituted imidazo[1,2-a]pyridine-3-carboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

The present application relates to novel 6-substituted imidazo[1,2-a]pyridine-3-carboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be classified into two groups either by structural features or by the type of ligands: the particulate guanylate cyclases which can be stimulated by natriuretic peptides, and the soluble guanylate cyclases which can be stimulated by NO. The soluble guanylate cyclases consist of two subunits and very probably contain one haem per heterodimer, which is part of the regulatory centre. This is of central importance for the activation mechanism. NO is able to bind to the iron atom of haem and thus markedly increase the activity of the enzyme. Haem-free preparations cannot, by contrast, be stimulated by NO. Carbon monoxide (CO) is also able to bind to the central iron atom of haem, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the aforementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase, use has exclusively been made of compounds such as organic nitrates whose effect is based on NO. The latter is formed by bioconversion and activates soluble guanylate cyclase by attack at the central iron atom of haem. In addition to the side effects, the development of tolerance is one of the crucial disadvantages of this mode of treatment.

In recent years, some substances have been described which stimulate soluble guanylate cyclase directly, i.e. without prior release of NO, such as, for example, 3-(5′-hydroxymethyl-2′-furyl)-1-benzylindazole [YC-1; Wu et al., Blood 84 (1994), 4226; Milsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Various imidazo[1,2-a]pyridine derivatives which can be used for treating disorders are described, inter alia, in EP 0 266 890-A1, WO 89/03833-A1, JP 01258674-A [cf. Chem. Abstr. 112:178986], WO 96/34866-A1, EP 1 277 754-A1, WO 2006/015737-A1, WO 2008/008539-A2, WO 2008/082490-A2, WO 2008/134553-A1, WO 2010/030538-A2, WO 2011/113606-A1 and WO 2012/165399-A1.

It was an object of the present invention to provide novel substances which act as stimulators of soluble guanylate cyclase and are suitable as such for treatment and/or prophylaxis of diseases.

The present invention provides compounds of the general formula (I)

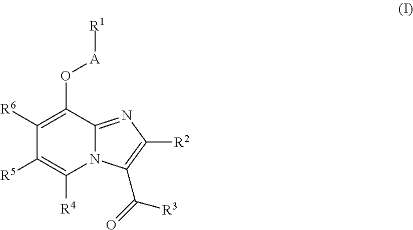

in which
A represents CH₂, CD₂ or CH(CH₃),
R¹ represents (C₃-C₇)-cycloalkyl, pyridyl or phenyl,
   where (C₃-C₇)-cycloalkyl may be substituted by 1 to 4 substituents independently of one another selected from the group consisting of fluorine, trifluoromethyl and (C₁-C₄)-alkyl,
   and
   where phenyl may be substituted by 1 to 4 substituents independently of one another selected from the group consisting of halogen, cyano, monofluoromethyl, difluoromethyl, trifluoromethyl, (C₁-C₄)-alkyl, (C₃-C₅)-cycloalkyl, (C₁-C₄)-alkoxy, difluoromethoxy and trifluoromethoxy,
   where pyridyl may be substituted by 1 to 4 substituents independently of one another selected from the group consisting of fluorine, chlorine, monofluoromethyl, difluoromethyl, trifluoromethyl and (C₁-C₄)-alkyl,
R² represents hydrogen, chlorine, (C₁-C₄)-alkyl, (C₁-C₄)-alkoxy, cyclopropyl, cyclobutyl, monofluoromethyl, difluoromethyl or trifluoromethyl,
   where (C₁-C₄)-alkyl may be substituted by (C₁-C₄)-alkoxy,
   where cyclopropyl and cyclobutyl may be up to disubstituted by fluorine,
R³ represents a group of the formula

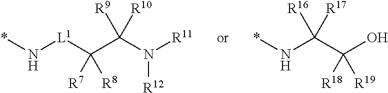

where
* represents the point of attachment to the carbonyl group,
L¹ represents a bond or (C₁-C₄)-alkanediyl,
R⁷ represents hydrogen, fluorine or (C₁-C₆)-alkyl,
   in which (C₁-C₆)-alkyl may be substituted up to five times by fluorine,
R⁸ represents hydrogen, fluorine, methyl or ethyl,
R⁹ represents hydrogen or (C₁-C₆)-alkyl,
   in which (C₁-C₆)-alkyl may be substituted up to five times by fluorine,
   in which (C₁-C₆)-alkyl may be substituted by trimethylsilyl,
R¹⁰ represents hydrogen or (C₁-C₄)-alkyl,
R¹¹ represents hydrogen or (C₁-C₃)-alkyl,
R¹² represents hydrogen or (C₁-C₃)-alkyl,
R¹⁶ represents hydrogen, (C₁-C₆)-alkyl or 5-membered heteroaryl,
   in which (C₁-C₆)-alkyl may be substituted up to five times by fluorine,
   in which 5-membered heteroaryl is substituted by methyl, difluoromethyl or trifluoromethyl,
R¹⁷ represents hydrogen or methyl,
R¹⁸ represents hydrogen or (C₁-C₄)-alkyl,
   in which (C₁-C₆)-alkyl may be substituted up to five times by fluorine,
R¹⁹ represents hydrogen or (C₁-C₄)-alkyl,
R⁴ represents hydrogen,
R⁵ represents hydrogen, chlorine, (C₁-C₄)-alkyl, (C₂-C₄)-alkynyl, (C₃-C₅)-cycloalkyl, difluoromethoxy, trifluoromethoxy or 5- or 6-membered heteroaryl,
   where (C₁-C₄)-alkyl may be substituted by (C₁-C₄)-alkoxy,
R⁶ represents hydrogen,
   and the N-oxides, salts, solvates, salts of the N-oxides and solvates of the N-oxides and salts thereof.

The present invention provides compounds of the general formula (I)

in which
A represents CH₂, CD₂ or CH(CH₃),
R¹ is (C₃-C₇)-cycloalkyl, pyridyl or phenyl,
   where (C₃-C₇)-cycloalkyl may be substituted by 1 to 4 substituents independently of one another selected from the group consisting of fluorine, trifluoromethyl and (C₁-C₄)-alkyl,
   and
   where phenyl may be substituted by 1 to 4 substituents independently of one another selected from the group consisting of halogen, cyano, monofluoromethyl, difluoromethyl, trifluoromethyl, (C₁-C₄)-alkyl, (C₃-C₅)-cycloalkyl, (C₁-C₄)-alkoxy, difluoromethoxy and trifluoromethoxy,
   where pyridyl may be substituted by 1 to 4 substituents independently of one another selected from the group consisting of fluorine, chlorine, monofluoromethyl, difluoromethyl, trifluoromethyl and (C₇-C₄)-alkyl,
R² represents hydrogen, (C₁-C₄)-alkyl, cyclopropyl, cyclobutyl, monofluoromethyl, difluoromethyl or trifluoromethyl,
   where (C₁-C₄)-alkyl may be substituted by (C₁-C₄)-alkoxy,
R³ represents a group of the formula

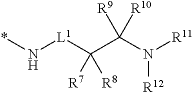

where
* represents the point of attachment to the carbonyl group,
L¹ represents a bond or (C₁-C₄)-alkanediyl,
R⁷ represents hydrogen, fluorine or (C₁-C₆)-alkyl,
   in which (C₁-C₆)-alkyl may be substituted up to five times by fluorine,
R⁸ represents hydrogen, fluorine, methyl or ethyl,
R⁹ represents hydrogen or (C₁-C₆-alkyl,
   in which (C₁-C₆)-alkyl may be substituted up to five times by fluorine,
R¹⁰ represents hydrogen or (C₁-C₄)-alkyl,
R¹¹ represents hydrogen or (C₁-C₃)-alkyl,
R¹² represents hydrogen or (C₁-C₃)-alkyl,
R⁴ represents hydrogen,
R⁵ represents hydrogen, chlorine, (C₁-C₄)-alkyl, (C₂-C₄)-alkynyl, (C₃-C₅)-cycloalkyl, difluoromethoxy, tri fluoromethoxy or 5- or 6-membered heteroaryl,
   where (C₁-C₄)-alkyl may be substituted by (C₁-C₄)-alkoxy,
R⁶ represents hydrogen,
   and the N-oxides, salts, solvates, salts of the N-oxides and solvates of the N-oxides and salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are mentioned below as working examples and the salts, solvates and solvates of the salts thereof if the compounds that are encompassed by formula (I) and are mentioned below are not already salts, solvates and solvates of the salts.

Preferred salts in the context of the present invention are physiologically acceptable salts of the compounds of the invention. Also encompassed are salts which are not themselves suitable for pharmaceutical applications but can be used, for example, for isolation or purification of the compounds of the invention.

Physiologically acceptable salts of the compounds of the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatographic processes are preferably used for this purpose, especially HPLC chromatography on an achiral or chiral phase.

If the compounds of the invention can occur in tautomeric forms, the present invention encompasses all the tautomeric forms.

The present invention also encompasses all suitable isotopic variants of the compounds of the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting materials.

The present invention additionally also encompasses prodrugs of the compounds of the invention. The term “prodrugs” in this context refers to compounds which may themselves be biologically active or inactive but are reacted (for example metabolically or hydrolytically) to give compounds of the invention during their residence time in the body.

In the context of the present invention, unless specified otherwise, the substituents are defined as follows:

Alkyl in the context of the invention is a straight-chain or branched alkyl radical having the particular number of carbon atoms specified. The following may be mentioned by way of example and by way of preference: methyl, ethyl, n-propyl, isopropyl, n-butyl, isobutyl, 1-methylpropyl, tert-butyl, n-pentyl, isopentyl, 1-ethylpropyl, 1-methybutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl, 2-ethylbutyl.

Carbocyclus or cycloalkyl in the context of the invention is a mono- or bicyclic saturated and partially unsaturated carbocycle having the number of ring carbon atoms stated in each case and up to 3 double bonds. The following may be mentioned by way of example and by way of preference: cyclopropyl, cyclobutyl, cyclopentyl, cyclohexyl, cycloheptyl, cyclopentenyl, cyclohexenyl, cyclohexadienyl, cycloheptenyl, cycloheptadienyl, indanyl, tetralinyl.

Alkenyl in the context of the invention is a straight-chain or branched alkenyl radical having 2 to 6 carbon atoms and one or two double bonds. Preference is given to a straight-chain or branched alkenyl radical having 2 to 4 carbon atoms and one double bond. The following may be mentioned by way of example and by way of preference: vinyl, allyl, isopropenyl and n-but-2-en-1-yl.

Alkynyl in the context of the invention is a straight-chain or branched alkynyl radical having 2 to 6 carbon atoms and one triple bond. The following may be mentioned by way of example and by way of preference: ethynyl, n-prop-1-yn-1-yl, n-prop-2-yn-1-yl, n-but-2-yn-1-yl and n-but-3-yn-1-yl.

Alkanediyl in the context of the invention is a straight-chain or branched divalent alkyl radical having 1 to 4 carbon atoms. The following may be mentioned by way of example and by way of preference: methylene, 1,2-ethylene, ethane-1,1-diyl, 1,3-propylene, propane-1,1-diyl, propane-1,2-diyl, propane-2,2-diyl, 1,4-butylene, butane-1,2-diyl, butane-1,3-diyl and butane-2,3-diyl.

Alkoxy in the context of the invention is a straight-chain or branched alkoxy radical having 1 to 4 carbon atoms. The following may be mentioned by way of example and by way of preference: methoxy, ethoxy, n-propoxy, isopropoxy, 1-methylpropoxy, n-butoxy, isobutoxy and tert-butoxy.

Alkoxycarbonyl in the context of the invention is a straight-chain or branched aikoxy radical having 1 to 4 carbon atoms and a carbonyl group attached to the oxygen. The following may be mentioned by way of example and by way of preference: methoxycarbonyl, ethoxycarbonyl, n-propoxycarbonyl, isopropoxycarbonyl and tert-butoxycarbonyl.

Alkylsulphonyl in the context of the invention is a straight-chain or branched alkyl radical which has 1 to 4 carbon atoms and is bonded via a sulphonyl group. Preferred examples include: methylsulphonyl, ethylsulphonyl, n-propylsulphonyl, isopropylsulphonyl, n-butylsulphonyl and tert-butylsulphonyl.

A 4- to 7-membered heterocycle in the context of the invention is a monocyclic saturated heterocycle which has a total of 4 to 7 ring atoms, contains one or two ring heteroatoms from the group consisting of N, C, S, SO and SO₂ and is joined via a ring carbon atom or any ring nitrogen atom. The following may be mentioned by way of example: azetidinyl, oxetanyl, pyrrolidinyl, pyrazolidinyl, tetrahydrofuranyl, thiolanyl, piperidinyl, piperazinyl, tetrahydropyranyl, tetrahydrothiopyranyl, morpholinyl, thiomorpholinyl, hexahydroazepinyl and hexahydro-1,4-diazepinyl. Preference is given to azetidinyl, oxetanyl, pyrrolidinyl, tetrahydrofuranyl, piperidinyl, piperazinyl, tetrahydropyranyl and morpholinyl.

Heteroaryl in the context of the invention is a monocyclic aromatic heterocycle (heteroaromatic) which has a total of 5 or 6 ring atoms, contains up to three identical or different ring heteroatoms from the group consisting of N, O and S and is joined via a ring carbon atom or via any ring nitrogen atom. The following may be mentioned by way of example and by way of preference: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl.

Halogen in the context of the invention includes fluorine, chlorine, bromine and iodine. Preference is given to chlorine or fluorine.

In the formula of the group that R³ or R¹ may represent, the end point of the line marked by the symbol * and # does not represent a carbon atom or a CH₂ group but is part of the bond to the respective atom to which R³ or R¹ is attached.

When radicals in the compounds of the invention are substituted, the radicals may be mono- or polysubstituted, unless specified otherwise. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, preference is given to compounds of the formula (I) in which
A represents CH₂,
R¹ represents pyridyl,
   where pyridyl may be substituted by 1 to 3 substituents independently of one another selected from the group consisting of fluorine, difluoromethyl, trifluoromethyl and methyl,
R² represents hydrogen, (C₁-C₄)-alkyl, cyclopropyl, cyclobutyl, monofluoromethyl, difluoromethyl or trifluoromethyl,
   where (C₁-C₄)-alkyl may be substituted by (C₁-C₄)-alkoxy,
R³ represents a group of the formula

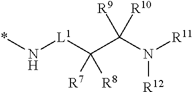

where
* represents the point of attachment to the carbonyl group,
L¹ represents a bond or (C₁-C₄)-alkanediyl,
R⁷ represents hydrogen, fluorine or (C₁—C)-alkyl,
   in which (C₁-C₆)-alkyl may be substituted up to five times by fluorine,
R⁸ represents hydrogen, fluorine, methyl or ethyl,
R⁹ represents hydrogen or (C₁-C₆-alkyl,
   in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethyl, ethynyl, cyclopropyl, difluoromethoxy, trifluoromethoxy or 5- or 6-membered heteroaryl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a pyridyl group of the formula

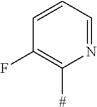

where
# represents the point of attachment to A,
R² represents methyl,
R³ represents a group of the formula

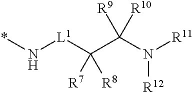

where
* represents the point of attachment to the carbonyl group,
L¹ represents a bond or methanediyl,
R⁷ represents hydrogen or fluorine,
R⁸ represents hydrogen or fluorine,
R⁹ represents hydrogen or (C₁-C₄)-alkyl,
in which (C₁-C₄)-alkyl may be substituted up to five times by fluorine,
   R¹⁰ represents hydrogen or methyl,
   R¹¹ represents hydrogen,
   R¹² represents hydrogen,
R⁴ represents hydrogen,
R⁵ represents hydrogen, chlorine, methyl, ethyl, cyclopropyl, difluoromethoxy, pyridyl, 1H-pyrazol-1-yl, 1-methyl-1H-pyrazol-4-yl or 1,3-oxazol-5-yl,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a pyridyl group of the formula

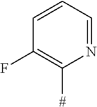

where
# represents the point of attachment to A,
R² represents methyl,
R³ represents a group of the formula

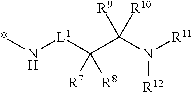

where
* represents the point of attachment to the carbonyl group,
L¹ represents a bond,
R⁷ represents hydrogen,
R⁸ represents hydrogen,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents hydrogen, chlorine, methyl or cyclopropyl,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ is cyclohexyl, pyridyl or phenyl,
   where phenyl may be substituted by 1 to 4 substituents independently of one another selected from the group consisting of fluorine, chlorine, bromine and methyl,
   and
   where pyridyl may be substituted by 1 to 3 substituents independently of one another selected from the group consisting of fluorine, chlorine, bromine and methyl,
R² represents chlorine, (C₁-C₄)-alkyl, methoxy or cyclobutyl,
   where (C₁-C₄)-alkyl is substituted by (C₁-C₄)-alkoxy,
   where cyclobutyl is disubstituted by fluorine,
R³ represents a group of the formula

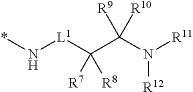

where
* represents the point of attachment to the carbonyl group,
L¹ represents a bond or (C₁-C₄)-alkanediyl,
R⁷ represents hydrogen or fluorine,
R⁸ represents hydrogen or fluorine,
R⁹ represents hydrogen or (C₁-C₆)-alkyl,
   in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethyl, ethynyl, cyclopropyl, difluoromethoxy or 5- or 6-membered heteroaryl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

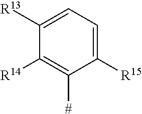

where
# represents the point of attachment to A,
and
R¹³ represents hydrogen or fluorine,
R¹⁴ and R¹⁵ represent fluorine,
R² represents methyl or chlorine,
   where methyl is substituted by methoxy,
R³ represents a group of the formula

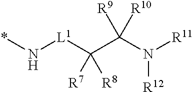

where
* represents the point of attachment to the carbonyl group,
L¹ represents a bond or methanediyl,
R⁷ represents hydrogen or fluorine,
R⁸ represents hydrogen or fluorine,
R¹⁰ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents chlorine, methyl, ethyl, cyclopropyl, difluoromethoxy, pyridyl, 1H-pyrazol-1-yl, 1-methyl-1H-pyrazol-4-yl or 1,3-oxazol-5-yl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

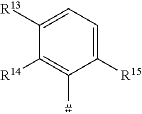

where
# represents the point of attachment to A,
and
R¹³ represents hydrogen or fluorine,
R¹⁴ and R¹⁵ represent fluorine,
R² represents methyl or chlorine,
   where methyl is substituted by methoxy,
R³ represents a group of the formula

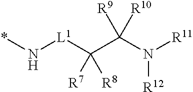

where
* represents the point of attachment to the carbonyl group,
L¹ represents a bond,
R⁷ represents hydrogen,
R⁸ represents hydrogen,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents chlorine, methyl or cyclopropyl,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ is cyclohexyl, pyridyl or phenyl,
   where phenyl may be substituted by 1 to 4 substituents independently of one another selected from the group consisting of fluorine, chlorine, bromine and methyl,
   and
   where pyridyl may be substituted by 1 to 3 substituents independently of one another selected from the group consisting of fluorine, chlorine, bromine and methyl,
R² represents (C₁-C₄)-alkyl,
   where (C₁-C₄)-alkyl is substituted by (C₁-C₄)-alkoxy,
R³ represents a group of the formula

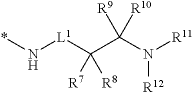

where
* represents the point of attachment to the carbonyl group,
L¹ represents a bond or (C₁-C₄)-alkanediyl,
R⁷ represents hydrogen or fluorine,
R⁸ represents hydrogen or fluorine,
R⁹ represents hydrogen or (C₁-C₆)-alkyl,
   in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethyl, ethynyl, cyclopropyl, difluoromethoxy or 5- or 6-membered heteroaryl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

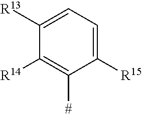

where
# represents the point of attachment to A,
and
R¹³ represents hydrogen or fluorine,
R¹⁴ and R¹⁵ represent fluorine,
R² represents methyl,
   where methyl is substituted by methoxy,
R³ represents a group of the formula

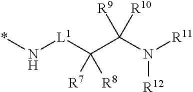

where
* represents the point of attachment to the carbonyl group,
L¹ represents a bond or methanediyl,
R⁷ represents hydrogen or fluorine,
R⁸ represents hydrogen or fluorine,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents chlorine, methyl, ethyl, cyclopropyl, difluoromethoxy, pyridyl, 1H-pyrazol-1-yl, 1-methyl-1H-pyrazol-4-yl or 1,3-oxazol-5-yl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

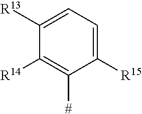

where
# represents the point of attachment to A,
and
R¹³ represents hydrogen or fluorine,
R¹⁴ and R¹⁵ represent fluorine,
R² represents methyl,
   where methyl is substituted by methoxy,
R³ represents a group of the formula

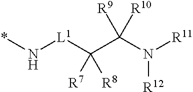

where
* represents the point of attachment to the carbonyl group,
L¹ represents a bond,
R⁷ represents hydrogen,
R⁸ represents hydrogen,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents chlorine, methyl or cyclopropyl,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents cyclohexyl, pyridyl or phenyl,
   where phenyl may be substituted by 1 to 4 substituents independently of one another selected from the group consisting of fluorine, chlorine, bromine and methyl,
   where pyridyl may be substituted by 1 to 3 substituents independently of one another selected from the group consisting of fluorine, chlorine, bromine and methyl,
R² represents hydrogen, chlorine, (C₁-C₄)-alkyl, methoxy, cyclopropyl, cyclobutyl, monofluoromethyl, difluoromethyl or trifluoromethyl,
   where (C₁-C₄)-alkyl may be substituted by (C₁-C₄)-aikoxy,
R³ represents a group of the formula

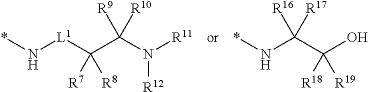

where
* represents the point of attachment to the carbonyl group,
L¹ represents (C₁-C₄)-alkanediyl,
R⁷ represents fluorine,
R⁸ represents fluorine,
R⁹ represents hydrogen or (C₁-C₆)-alkyl,
   in which (C₁-C₆)-alkyl may be substituted up to five times by fluorine,
   in which (C₁-C₆)-alkyl may be substituted by trimethylsilyl,
R¹⁰ represents hydrogen or (C₁-C₄)-alkyl,
R¹¹ represents hydrogen,
R¹² represents hydrogen,
R¹⁶ represents hydrogen, (C₁-C₆)-alkyl or 5-membered heteroaryl,
   in which (C₁-C₆)-alkyl may be substituted up to five times by fluorine,
   in which 5-membered heteroaryl is substituted by methyl, difluoromethyl or trifluoromethyl,
R¹⁷ represents hydrogen or methyl,
R¹⁸ represents hydrogen or (C₁-C₄)-alkyl,
   in which (C₁-C₆)-alkyl may be substituted up to five times by fluorine,
R¹⁹ represents hydrogen or (C₁-C₄)-alkyl,
R⁴ represents hydrogen,
R⁵ represents hydrogen, chlorine, methyl, ethyl, ethynyl, cyclopropyl, difluoromethoxy or 5- or 6-membered heteroaryl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

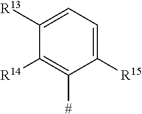

where
# represents the point of attachment to A,
and
R¹³ represents hydrogen or fluorine,
R¹⁴ and R¹⁵ represent fluorine,
R² represents methyl or methoxy,
R³ represents a group of the formula

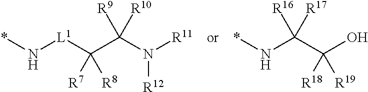

where
* represents the point of attachment to the carbonyl group,
L¹ represents methanediyl or ethanediyl,
R⁷ represents fluorine,
R⁸ represents fluorine,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R¹⁶ represents butyl,
   in which butyl may be substituted up to five times by fluorine,
R¹⁷ represents hydrogen,
R¹⁸ represents hydrogen or methyl,
R¹⁹ represents hydrogen or methyl,
R⁴ represents hydrogen,
R⁵ represents chlorine, methyl, ethyl, cyclopropyl, difluoromethoxy, pyridyl, 1H-pyrazol-1-yl, 1-methyl-1H-pyrazol-4-yl or 1,3-oxazol-5-yl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents cyclohexyl, pyridyl or phenyl,
   where phenyl may be substituted by 1 to 4 substituents independently of one another selected from the group consisting of fluorine, chlorine, bromine and methyl,
   where pyridyl may be substituted by 1 to 3 substituents independently of one another selected from the group consisting of fluorine, chlorine, bromine and methyl,
R² represents hydrogen, (C₁-C₄)-alkyl, cyclopropyl, cyclobutyl, monofluoromethyl, difluoromethyl or trifluoromethyl,
   where (C₁-C₄)-alkyl may be substituted by (C₁-C₄)-alkoxy,
R³ represents a group of the formula

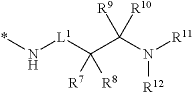

where
* represents the point of attachment to the carbonyl group,
L¹ represents (C₁-C₄)-alkanediyl,
R⁷ represents fluorine,
R⁸ represents fluorine,
R⁹ represents hydrogen or (C₁-C₆)-alkyl,
   in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethyl, ethynyl, cyclopropyl, difluoromethoxy or 5- or 6-membered heteroaryl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

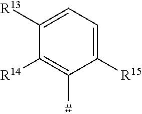

where
# represents the point of attachment to A,
and
R¹³ represents hydrogen or fluorine,
R¹⁴ and R¹⁵ represent fluorine,
R² represents methyl,
R³ represents a group of the formula

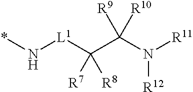

where
* represents the point of attachment to the carbonyl group,
L¹ represents methanediyl or ethanediyl,
R⁷ represents fluorine,
R⁸ represents fluorine,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents chlorine, methyl, ethyl, cyclopropyl, difluoromethoxy, pyridyl, 1H-pyrazol-1-yl, 1-methyl-1H-pyrazol-4-yl or 1,3-oxazol-5-yl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

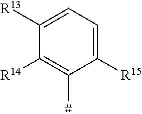

where
# represents the point of attachment to A,
and
R¹³ represents hydrogen or fluorine,
R¹⁴ and R¹⁵ represent fluorine,
R² represents methyl,
R³ represents a group of the formula

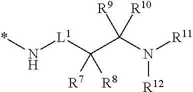

where
* represents the point of attachment to the carbonyl group,
L¹ represents methanediyl or ethanediyl,
R⁷ represents fluorine,
R⁸ represents fluorine,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents chlorine, methyl or cyclopropyl,
   where methyl may be substituted by a methoxy substituent,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents cyclohexyl, pyridyl or phenyl,
   where phenyl may be substituted by 1 to 4 substituents independently of one another selected from the group consisting of fluorine, chlorine, bromine and methyl,
   and
   where pyridyl may be substituted by 1 to 3 substituents independently of one another selected from the group consisting of fluorine, chlorine, bromine and methyl,
R² represents hydrogen, (C₁-C₄)-alkyl, cyclopropyl, cyclobutyl, monofluoromethyl, difluoromethyl or trifluoromethyl,
   where (C₁-C₄)-alkyl may be substituted by (C₁-C₄)-alkoxy,
R³ represents a group of the formula

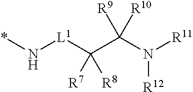

where
* represents the point of attachment to the carbonyl group,
L¹ represents a bond or (C₁-C₄)-alkanediyl,
R⁷ represents hydrogen, fluorine or (C₁-C₆)-alkyl,
   in which (C₁-C₆)-alkyl may be substituted up to five times by fluorine,
R⁸ represents hydrogen, fluorine, methyl or ethyl,
R⁹ represents hydrogen or (C₁-C₆)-alkyl,
   in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethynyl, cyclopropyl, difluoromethoxy, pyridyl, 1H-pyrazol-1-yl, 1-methyl-1H-pyrazol-4-yl or 1,3-oxazol-5-yl,
   where methyl is substituted by methoxy,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

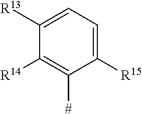

where
# represents the point of attachment to A.
and
R¹³ represents hydrogen or fluorine,
R¹⁴ and R¹⁵ represent fluorine,
R² represents methyl,
R³ represents a group of the formula

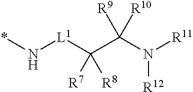

where
* represents the point of attachment to the carbonyl group,
L¹ represents a bond or methanediyl,
R⁷ represents hydrogen or fluorine,
R⁸ represents hydrogen or fluorine,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents chlorine, methyl, cyclopropyl, difluoromethoxy, pyridyl, 1H-pyrazol-1-yl, 1-methyl-1H-pyrazol-4-yl or 1,3-oxazol-5-yl,
   where methyl is substituted by methoxy,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

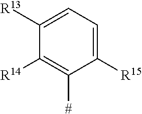

where
# represents the point of attachment to A,
and
R¹³ represents hydrogen or fluorine,
R¹⁴ and R¹⁵ represent fluorine,
R² represents methyl,
R³ represents a group of the formula

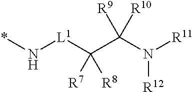

where
* represents the point of attachment to the carbonyl group,
L¹ represents a bond,
R⁷ represents hydrogen,
R⁸ represents hydrogen,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R⁴ represents hydrogen,
R⁵ represents cyclopropyl, 1H-pyrazol-1-yl, 1-methyl-1H-pyrazol-4-yl or 1,3-oxazol-5-yl,
R⁶ represents hydrogen,
   and the N-oxides, salts, solvates, salts of the N-oxides and solvates of the N-oxides and salts thereof.

In the context of the present invention, preference is also given to compounds of the formula (I) in which
A represents CH₂,
   and the N-oxides, salts, solvates, salts of the N-oxides and solvates of the N-oxides and salts thereof.

In the context of the present invention, preference is also given to compounds of the formula (I) in which
A represents CH₂,
R¹ represents pyridyl or phenyl,
   where phenyl may be substituted by 1 to 4 substituents independently of one another selected from the group consisting of fluorine, chlorine, bromine and methyl,
   and
   where pyridyl may be substituted by 1 to 3 substituents independently of one another selected from the group consisting of fluorine, chlorine, bromine and methyl,
      and the N-oxides, salts, solvates, salts of the N-oxides and solvates of the N-oxides and salts thereof

In the context of the present invention, preference is also given to compounds of the formula (I) in which
R¹ represents pyridyl,
   where pyridyl may be substituted by 1 to 3 substituents independently of one another selected from the group consisting of fluorine, difluoromethyl, trifluoromethyl and methyl,
      and the N-oxides, salts, solvates, salts of the N-oxides and solvates of the N-oxides and salts thereof.

In the context of the present invention, preference is also given to compounds of the formula (I) in which
R¹ represents a pyridyl group of the formula

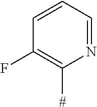

where
# represents the point of attachment to A,
   and the N-oxides, salts, solvates, salts of the N-oxides and solvates of the N-oxides and salts thereof.

In the context of the present invention, preference is also given to compounds of the formula (I) in which
R¹ represents a phenyl group of the formula

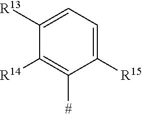

where
# represents the point of attachment to A,
and
R¹³ represents hydrogen or fluorine,
R¹⁴ and R¹⁵ represent fluorine,
   and the N-oxides, salts, solvates, salts of the N-oxides and solvates of the N-oxides and salts thereof.

In the context of the present invention, preference is also given to compounds of the formula (I) in which
R¹ represents a phenyl group of the formula

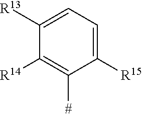

where
# represents the point of attachment to A,
and
R¹³ represents hydrogen,
R¹⁴ and R¹⁵ represent fluorine,
   and the N-oxides, salts, solvates, salts of the N-oxides and solvates of the N-oxides and salts thereof.

In the context of the present invention, preference is also given to compounds of the formula (I) in which
R² represents (C₁-C₄)-alkyl
   where (C₁-C₄)-alkyl is substituted by (C₁-C₄)-alkoxy,
      and the N-oxides, salts, solvates, salts of the N-oxides and solvates of the N-oxides and salts thereof.

In the context of the present invention, preference is also given to compounds of the formula (I) in which
R² represents methyl,
   where methyl is substituted by methoxy,
      and the N-oxides, salts, solvates, salts of the N-oxides and solvates of the N-oxides and salts thereof.

In the context of the present invention, preference is also given to compounds of the formula (I) in which
R² represents methyl,
   and the N-oxides, salts, solvates, salts of the N-oxides and solvates of the N-oxides and salts thereof.

In the context of the present invention, preference is also given to compounds of the formula (I) in which
R² represents chlorine,
   and the N-oxides, salts, solvates, salts of the N-oxides and solvates of the N-oxides and salts thereof.

In the context of the present invention, preference is also given to compounds of the formula (I) in which
R² represents methoxy,
   and the N-oxides, salts, solvates, salts of the N-oxides and solvates of the N-oxides and salts thereof.

In the context of the present invention, preference is also given to compounds of the formula (I) in which
R³ represents a group of the formula

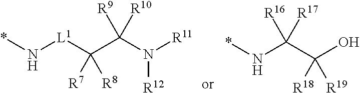

where
* represents the point of attachment to the carbonyl group,
L¹ represents methanediyl or ethanediyl,
R⁷ represents fluorine,
R⁸ represents fluorine,
R⁹ represents hydrogen or (C₁-C₄)-alkyl,
   in which (C₁-C₄)-alkyl may be substituted up to five times by fluorine,
R¹⁰ represents hydrogen or methyl,
R¹¹ represents hydrogen,
R¹² represents hydrogen,
R¹⁶ represents butyl,
   in which butyl may be substituted up to five times by fluorine,
R¹⁷ represents hydrogen,
R¹⁸ represents hydrogen or methyl,
R¹⁹ represents hydrogen or methyl,
   and the N-oxides, salts, solvates, salts of the N-oxides and solvates of the N-oxides and salts thereof.

In the context of the present invention, preference is also given to compounds of the formula (I) in which
R³ represents a group of the formula

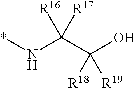

where
R¹⁶ represents butyl,
   in which butyl may be substituted up to five times by fluorine,
R¹⁷ represents hydrogen,
R¹⁸ represents hydrogen or methyl,
R¹⁹ represents hydrogen or methyl,
   and the N-oxides, salts, solvates, salts of the N-oxides and solvates of the N-oxides and salts thereof.

In the context of the present invention, preference is also given to compounds of the formula (I) in which
R³ represents a group of the formula

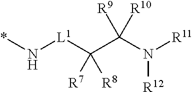

where
* represents the point of attachment to the carbonyl group,
L¹ represents a bond or (C₁-C₄)-alkanediyl,
R⁷ represents hydrogen or fluorine,
R⁸ represents hydrogen or fluorine,
R⁹ represents hydrogen or (C₁-C₆)-alkyl,
   in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
   and the N-oxides, salts, solvates, salts of the N-oxides and solvates of the N-oxides and salts thereof.

In the context of the present invention, preference is also given to compounds of the formula (I) in which
R³ represents a group of the formula

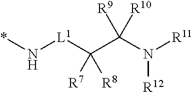

where
* represents the point of attachment to the carbonyl group,
L¹ represents a bond,
R⁷ represents hydrogen,
R⁸ represents hydrogen,
R⁹ represents hydrogen or (C₁-C₆)-alkyl,
   in which (C₁-C₆)-alkyl may be substituted up to five times by fluorine,
R¹⁰ represents hydrogen or methyl,
R¹¹ represents hydrogen,
R¹² represents hydrogen,
   and the N-oxides, salts, solvates, salts of the N-oxides and solvates of the N-oxides and salts thereof.

In the context of the present invention, preference is also given to compounds of the formula (I) in which
R³ represents a group of the formula

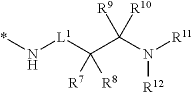

where
* represents the point of attachment to the carbonyl group,
L¹ represents methanediyl or ethanediyl,
R⁷ represents fluorine,
R⁸ represents fluorine,
R⁹ represents hydrogen or (C₁-C₆)-alkyl,
   in which (C₁-C₆)-alkyl may be substituted up to five times by fluorine,
R¹⁰ represents hydrogen or methyl,
R¹¹ represents hydrogen,
R¹² represents hydrogen,
   and the N-oxides, salts, solvates, salts of the N-oxides and solvates of the N-oxides and salts thereof.

In the context of the present invention, preference is also given to compounds of the formula (I) in which
R³ represents a group of the formula

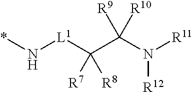

where
* represents the point of attachment to the carbonyl group,
L¹ represents methanediyl,
R⁷ represents fluorine,
R⁸ represents fluorine,
R⁹ represents hydrogen or (C₁-C₆)-alkyl,
   in which (C₁-C₆)-alkyl may be substituted up to five times by fluorine,
R¹⁰ represents hydrogen or methyl,
R¹¹ represents hydrogen,
R¹² represents hydrogen,
   and the N-oxides, salts, solvates, salts of the N-oxides and solvates of the N-oxides and salts thereof.

In the context of the present invention, preference is also given to compounds of the formula (I) in which
R³ represents a group of the formula

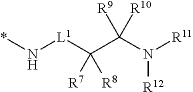

where
* represents the point of attachment to the carbonyl group,
L¹ represents methanediyl,
R⁷ represents fluorine,
R⁸ represents fluorine,
R⁹ represents hydrogen,
R¹⁰ represents hydrogen,
R¹¹ represents hydrogen,
R¹² represents hydrogen,
   and the N-oxides, salts, solvates, salts of the N-oxides and solvates of the N-oxides and salts thereof.

In the context of the present invention, preference is also given to compounds of the formula (I) in which
R⁵ represents chlorine, methyl, ethyl, cyclopropyl, difluoromethoxy, trifluoromethoxy or 5- or 6-membered heteroaryl,
   where methyl is substituted by methoxy,
      and the N-oxides, salts, solvates, salts of the N-oxides and solvates of the N-oxides and salts thereof.

In the context of the present invention, preference is also given to compounds of the formula (I) in which
R⁵ represents chlorine, methyl, cyclopropyl, difluoromethoxy, pyridyl, 1H-pyrazol-1-yl, 1-methyl-1H-pyrazol-4-yl or 1,3-oxazol-5-yl,
   where methyl is substituted by methoxy,
      and the N-oxides, salts, solvates, salts of the N-oxides and solvates of the N-oxides and salts thereof

In the context of the present invention, preference is also given to compounds of the formula (I) in which
R⁵ represents cyclopropyl, 1H-pyrazol-1-yl, 1-methyl-1H-pyrazol-4-yl or 1,3-oxazol-5-yl,
   and the N-oxides, salts, solvates, salts of the N-oxides and solvates of the N-oxides and salts thereof.

The invention further provides a process for preparing the compounds of the formula (I) according to the invention, characterized in that

### [A] A Compound of the Formula (II)

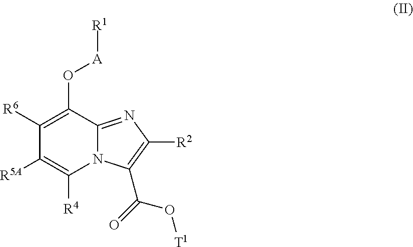

in which A, R¹, R², R⁴ and R⁶ are each as defined above,
R^{5A} has the meanings given for R⁵ or represents bromine,
and
T¹ represents (C₁-C₄)-alkyl or benzyl,
   is reacted in an inert solvent in the presence of a suitable base or acid to give a carboxylic acid of the formula (III)

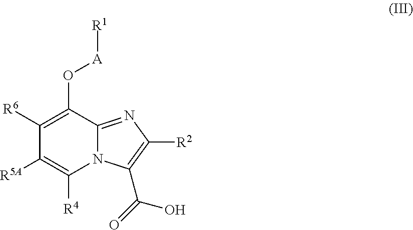

in which A, R¹, R², R⁴ and R⁶ are each as defined above,
and
R^{5A} has the meanings given for R⁵ or represents bromine,
   and this is subsequently reacted in an inert solvent under amide coupling conditions with an amine of the formula (IV)

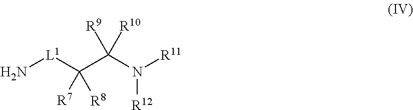

in which L¹, R⁷, R⁸, R⁹, R¹⁰, R¹¹ and R¹² each have the meanings given above,
and, if
R^{5A} represents bromine,
   the compounds are reacted in an inert solvent in the presence of a suitable transition metal catalyst, optionally in the presence of a suitable base, with a compound of the formula (IV-A)

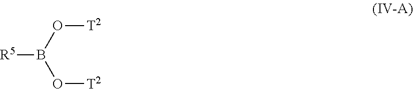

in which
R⁵ has the meaning given above
and
T² represents hydrogen or (C₁-C₄)-alkyl, or the two T² radicals together form a —C(CH₃)₂—C(CH₃)₂— bridge,
or

### [B] A Compound of the Formula (III-A)

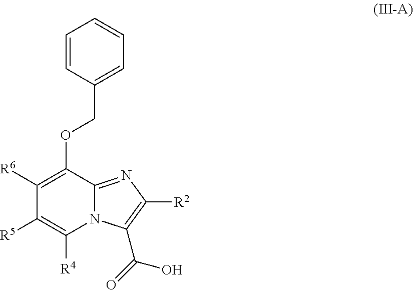

in which R², R⁴, R⁵ and R⁶ each have the meanings given above,
is converted in an inert solvent under amide coupling conditions with an amine of the formula (IV) into a compound of the formula (I-A)

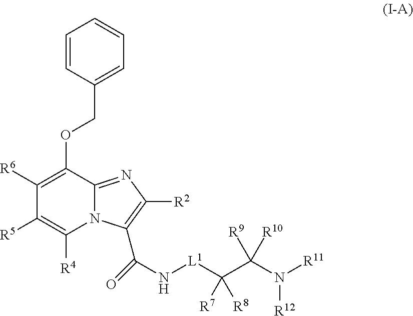

in which L¹, R², R⁴, R⁵, R⁶, R⁷, R⁸, R⁹, R¹⁰, R¹¹ and R¹² each have the meanings given above,
and the benzyl group is subsequently detached therefrom by the methods known to the person skilled in the art and the resulting compound of the formula (V)

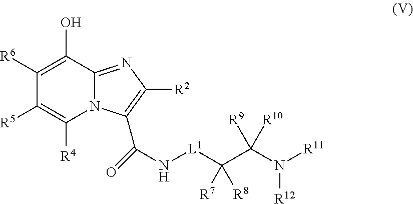

in which L¹, R², R⁴, R⁵, R⁶, R⁷, R⁸, R⁹, R¹⁰, R¹¹ and R¹² each have the meanings given above,
is reacted in an inert solvent in the presence of a suitable base with a compound of the formula (VI)

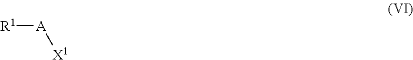

in which A and R have the meaning given above and
X¹ represents a suitable leaving group, in particular chlorine, bromine, iodine, mesylate, triflate or tosylate,
   then any protecting groups present are detached, and the resulting compounds of the formula (I) are optionally converted with the appropriate (i) solvents and/or (ii) acids or bases to the solvates, salts and/or solvates of the salts thereof.

The compounds of the formula (I-A) form a subgroup of compounds of the formula (I) according to the invention.

The preparation processes described can be illustrated by way of example by the following synthesis scheme (Scheme 1):

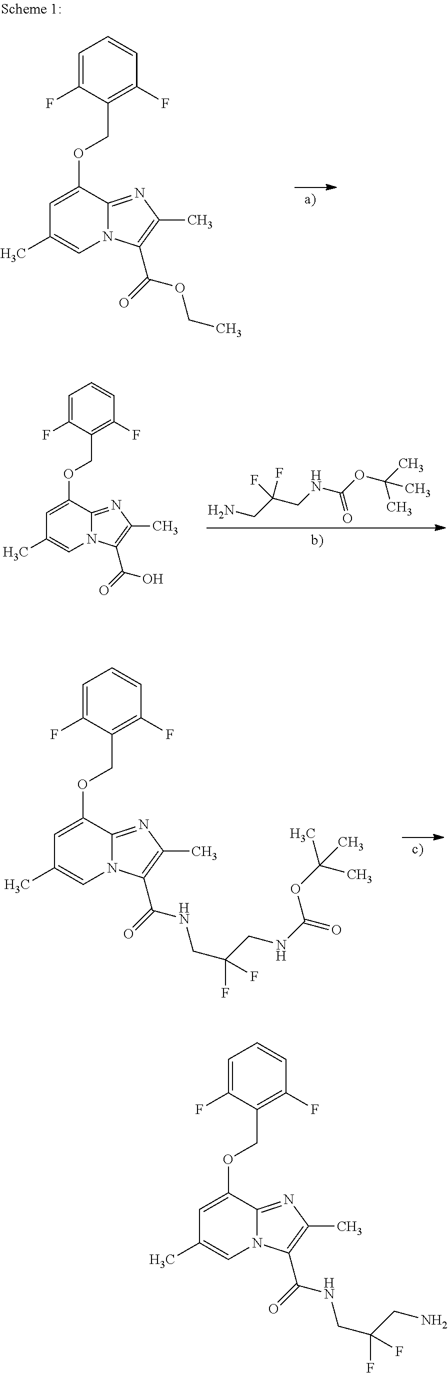

[a): lithium hydroxide, THF/methanol/H₂O, RT; b): HATU, 4-methylmorpholine or N,N-diisopropylethylamine, DMF; c): HCl, Et₂O or TFA, CH₂Cl₂].

The compounds of the formulae (IV) and (VI) are commercially available, known from the literature or can be prepared in analogy to literature processes.

The free bases of (IV) can be released from the compounds, optionally provided with an amino protective group, (IV), respectively, for example using acids such as hydrogen chloride and trifluoroacetic acid in suitable solvents such as diethyl ether, dichloromethane, 1,4-dioxane, water, methanol, ethanol and mixtures thereof,

Inert solvents for the process steps (III)+(IV)→(I) and (III-A)+(IV)→(I-A) are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, halohydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulphoxide, N,N-dimethylformamide, N,N-dimethylacetamide, N,N′-dimethylpropyleneurea (DMPU) or N-methylpyrrolidone (NMP). It is likewise possible to use mixtures of the solvents mentioned. Preference is given to dichloromethane, tetrahydrofuran, dimethylformamide or mixtures of these solvents.

Suitable for use as condensing agents for the amide formation in process steps (III)+(IV)→(I) and (III-A)+(IV)→(I-A) are, for example, carbodiimides such as N,N′-diethyl-, N,N′-dipropyl-, N,N′-diisopropyl-, N,N′-dicyclohexylcarbodiimde (DCC) or N-(3-dimethylaminopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydroquinoline or isobutyl chloroformate, propanephosphonic anhydride (T3P), 1-chloro-N,N,2-trimethylprop1-ene-1-amine, diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1, 1,3,3-tetramethyluronium tetrafluoroborate (TPTU), O-(7-azabenzotriaztrazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), optionally in combination with further auxiliaries such as 1-hydroxybenzotriazole (HOBt) or N-hydroxysuccinimide (HOSu), and also as bases alkali metal carbonates, for example sodium carbonate or potassium carbonate or sodium bicarbonate or potassium bicarbonate, or organic bases such as trialkylamines, for example triethylamine, N-methylmorpholine, N-methylpiperidine or N,N-diisopropylethylamine. Preference is given to using TBTU in combination with N-methylmorpholine, HATU in combination with N,N-diisopropylethylamine or 1-chloro-N,N,2-trimethylprop-1-en-1-amine.

The condensation (III)+(IV)→(I) and (III-A)+(IV)→(I-A) is generally conducted within a temperature range from −20° C. to +100° C., preferably at 0° C. to +60° C. The conversion can be carried out under atmospheric, elevated or reduced pressure (for example from 0.5 to 5 bar). In general, the reaction is carried out at atmospheric pressure.

Alternatively, the carboxylic acid of the formula (III) can also first be converted to the corresponding carbonyl chloride and the latter can then be converted directly or in a separate reaction with an amine of the formula (IV) to the compounds of the invention. The formation of carbonyl chlorides from carboxylic acids is effected by the methods known to those skilled in the art, for example by treatment with thionyl chloride, sulphuryl chloride or oxalyl chloride, in the presence of a suitable base, for example in the presence of pyridine, and optionally with addition of dimethylformamide, optionally in a suitable inert solvent.

The hydrolysis of the ester group T¹ in the compounds of the formula (II) is effected by customary methods, by treating the esters in inert solvents with acids or bases, in which latter case the salts formed at first are converted to the free carboxylic acids by treating with acid. In the case of the tert-butyl esters, the ester hydrolysis is preferably effected with acids. In the case of the benzyl esters, the ester hydrolysis is preferably effected by hydrogenolysis with palladium on activated carbon or Raney nickel. Suitable inert solvents for this reaction are water or the organic solvents customary for ester hydrolysis. These preferably include alcohols such as methanol, ethanol, n-propanol, isopropanol, n-butanol or tert-butanol, or ethers such as diethyl ether, tetrahydrofuran, 2-methyltetrahydrofuran, dioxane or glycol dimethyl ether, or other solvents such as acetone, dichloromethane, dimethylformamide or dimethyl sulphoxide. It is also possible to use mixtures of the solvents mentioned. In the case of a basic ester hydrolysis, preference is given to using mixtures of water with dioxane, tetrahydrofuran, methanol and/or ethanol.

Suitable bases for the ester hydrolysis are the customary inorganic bases. These preferably include alkali metal or alkaline earth metal hydroxides, for example sodium hydroxide, lithium hydroxide, potassium hydroxide or barium hydroxide, or alkali metal or alkaline earth metal carbonates, such as sodium carbonate, potassium carbonate or calcium carbonate. Particular preference is given to sodium hydroxide or lithium hydroxide.

Suitable acids for the ester cleavage are generally sulphuric acid, hydrogen chloride/hydrochloric acid, hydrogen bromide/hydrobromic acid, phosphoric acid, acetic acid, trifluoroacetic acid, toluenesulphonic acid, methanesulphonic acid or trifluoromethanesulphonic acid, or mixtures thereof, optionally with addition of water. Preference is given to hydrogen chloride or trifluoroacetic acid in the case of the tert-butyl esters and to hydrochloric acid in the case of the methyl esters.

The ester hydrolysis is generally carried out within a temperature range from 0° C. to +100° C., preferably at +0° C. to +50° C.

These conversions can be performed at atmospheric, elevated or reduced pressure (for example from 0.5 to 5 bar). In general, the reactions are in each case carried out at atmospheric pressure.

The coupling with (IV-A) is carried out in a solvent which is inert under the reaction conditions. Suitable solvents are, for example, alcohols such as methanol, ethanol, n-propanol, isopropanol, n-butanol or tert-butanol, ethers such as diethyl ether, dioxane, tetrahydrofuran, glycol dimethyl ether or diethylene glycol dimethyl ether, or other solvents such as 1,2-dimethoxyethane (DME), dimethylformamide (DMF), dimethyl sulphoxide (DMSO), N,N′-dimethylpropyleneurea (DMPU), N-methylpyrrolidone (NMP), pyridine, acetonitrile, toluene or else water. It is also possible to use mixtures of the solvents mentioned. Preference is given to ethanol, dimethoxyethane, dioxane, acetonitrile, toluene and water and mixtures of these solvents.

The conversion with (IV-A) can optionally be carried out in the presence of a suitable palladium and/or copper catalyst. A suitable palladium catalyst is, for example, palladium(II) acetate, tetrakis(triphenylphosphine)palladium(0), bis(tri-tert-butylphosphine)palladium(0), bis(triphenylphosphine)palladium(II) chloride, bis(acetonitrile)palladium(II) chloride and [1,1′-bis(diphenylphosphino)ferrocene]dichloropalladium(II) and the corresponding dichloromethane complex, optionally in conjunction with additional phosphane ligands, for example (2-biphenyl)di-tert-butylphosphine, 2-dicyclohexylphosphino-2′,6′-dimethoxybiphenyl (SPHOS), dicyclohexyl[2′,4′,6′-tris(1-methylethyl)biphenyl-2-yl]phosphane (XPHOS), 2-dicyclohexylphosphino-2′,6′-diisopropoxybiphenyl (RuPhos), bis(2-phenylphosphinophenyl) ether (DPEphos) or 4,5-bis(diphenylphosphino)-9,9-dimethylxanthene (Xantphos) [cf., for example, Hassan J. et al., Chem. Rev. 102, 1359-1469 (2002)].

The conversion with (IV-A) is optionally carried out in the presence of a suitable base. Suitable bases for this conversion are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or caesium carbonate, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide, sodium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO) or potassium phosphate. Preference is given to using potassium phosphate.

The reaction with (IV-A) is generally carried out in a temperature range from 0° C. to +200° C., preferably at from +80° C. to +150° C. The conversion can be carried out under atmospheric, elevated or reduced pressure (for example from 0.5 to 5 bar). In general, the reaction is carried out at atmospheric pressure.

Inert solvents for the process step (V)+(VI)→(I) are, for example, halohydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fi-actions, or other solvents such as acetone, methyl ethyl ketone, ethyl acetate, acetonitrile, N,N-dimethylformamide, N,N-dimethylacetamide, dimethyl sulphoxide, N,N′-dimethylpropyleneurea (DMPU), N-methylpyrrolidone (NMP) or pyridine. It is also possible to use mixtures of the solvents mentioned. Preference is given to using dimethylformamide or dimethyl sulphoxide.

Suitable bases for the process step (V)+(VI)→(I) are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or caesium carbonate, optionally with addition of an alkali metal iodide, for example sodium iodide or potassium iodide, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 4-(N,N-dimethylamino)pyridine (DMAP), 1,5-diazabicyclo[4.3.0]non-5-ene (DBN), 1,8-diazabicyclo[5.4.0]undec-7-ene (DBU) or 1,4-diazabicyclo[2.2.2]octane (DABCO( ). Preference is given to using potassium carbonate, caesium carbonate or sodium methoxide.

The reaction is generally effected within a temperature range from 0° C. to +120° C., preferably at +20° C. to +80° C., optionally in a microwave. The reaction can be carried out under atmospheric, elevated or reduced pressure (for example from 0.5 to 5 bar).

The amino protecting group used is preferably tert-butylcarbonyl (Boc) or benzyloxycarbonyl (Z). Protecting groups used for a hydroxyl or carboxyl function are preferably tert-butyl or benzyl.

These protecting groups are detached by customary methods, preferably by reaction with a strong acid such as hydrogen chloride, hydrogen bromide or trifluoroacetic acid in an inert solvent such as dioxane, diethyl ether, dichloromethane or acetic acid; it is optionally also possible to effect the detachment without an additional inert solvent. In the case of benzyl and benzyloxycarbonyl as protecting groups, these may also be removed by hydrogenolysis in the presence of a palladium catalyst. The detachment of the protecting groups mentioned can optionally be undertaken simultaneously in a one-pot reaction or in separate reaction steps.

The removal of the benzyl group in the reaction step (I-A)→(V) is carried out here by customary methods known from protecting group chemistry, preferably by hydrogenolysis in the presence of a palladium catalyst, for example palladium on activated carbon, in an inert solvent, for example ethanol or ethyl acetate [see also, for example, T. W. Greene and P. G. M. Wuts, Protective Groups in Organic Synthesis, Wiley, New York, 1999].

The compounds of the formula (II) are known from the literature or can be prepared by reacting a compound of the formula (VII)

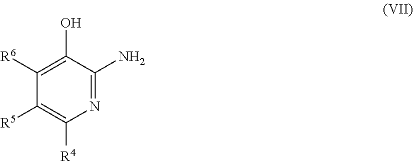

in which R⁴, R⁵ and R⁶ have the meaning given above,
in an inert solvent in the presence of a suitable base with a compound of the formula (VI) to give a compound of the formula (VIII)

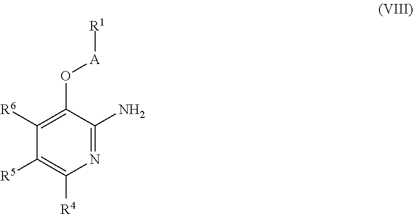

in which R¹, R⁴, R⁵ and R⁶ each have the meanings given above,
and then reacting the latter in an inert solvent with a compound of the formula (IX)

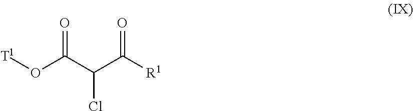

in which R² and T¹ are each as defined above.

The process described is illustrated in an exemplary manner by the scheme below (Scheme 2):

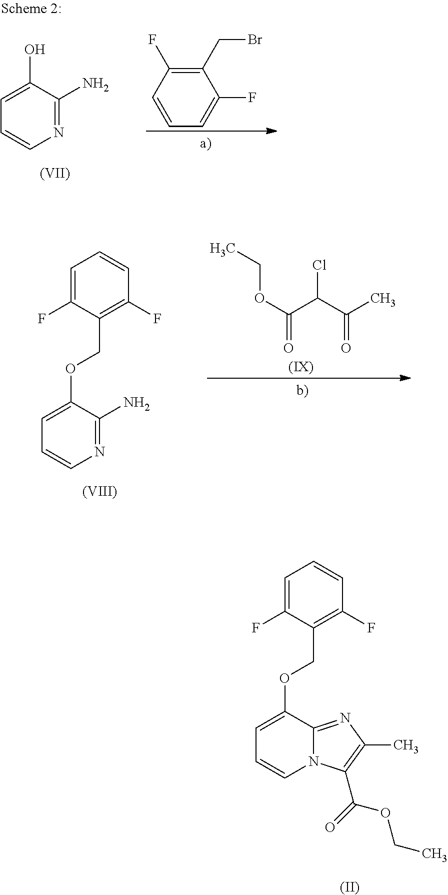

[a): i) NaOMe, MeOH, RT; ii) DMSO, RT; b): EtOH, molecular sieve, reflux].

The synthesis sequence shown can be modified such that the respective reaction steps are carried out in a different order. An example of such a modified synthesis sequence is shown in Scheme 3.

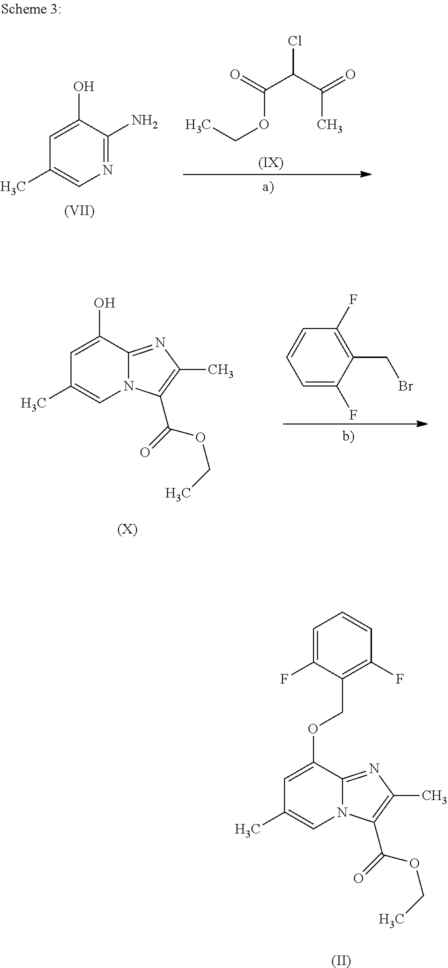

[a): EtOH, molecular sieve, reflux; b): b) Cs₂CO₃, DMF, 50° C.].

Inert solvents for the ring closure to give the imidazo[1,2-a]pyridine base skeleton (VIII)+(IX)→(II) or (VII)+(IX)→(X) are the customary organic solvents. These preferably include alcohols such as methanol, ethanol, n-propanol, isopropanol, n-butanol, n-pentanol or tert-butanol, or ethers such as diethyl ether, tetrahydrofuran, 2-methyltetrahydrofuran, dioxane or glycol dimethyl ether, or other solvents such as acetone, dichloromethane, 1,2-dichloroethane, acetonitrile, dimethylformamide or dimethyl sulphoxide. It is also possible to use mixtures of the solvents mentioned. Preference is given to using ethanol.

The ring closure is generally effected within a temperature range from +50° C. to +150° C., preferably at +50° C. to +100° C., optionally in a microwave.

The ring closure (VIII)+(IX)→(II) or (VII)+(IX)→(X) is optionally effected in the presence of dehydrating reaction additives, for example in the presence of molecular sieve (pore size 4 Å), or using a water separator. The reaction (VIII)+(IX)→(II) or (VII)+(IX)→(X) is effected using an excess of the reagent of the formula (IX), for example with 1 to 20 equivalents of the reagent (IX), optionally with addition of bases (for example sodium hydrogencarbonate), in which case this addition can be effected all at once or in several portions.

As an alternative to the introduction of R¹ by reaction of the compounds (V), (VII) or (X) with compounds of the formula (VI), as shown in Schemes 1 to 3, it is likewise possible—as shown in Scheme 4 to react these intermediates with alcohols of the formula (XI) under conditions of the Mitsunobu reaction.

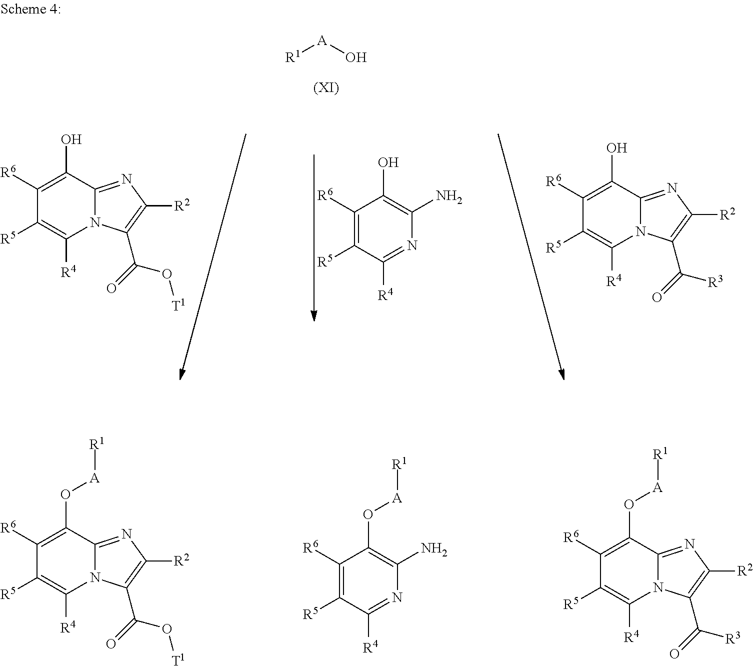

Typical reaction conditions for such Mitsunobu condensations of phenols with alcohols can be found in the relevant literature, e.g. Hughes, D. L. Org. React. 1992, 42, 335; Dembinski, R. Eur. J. Org. Chem. 2004, 2763. Typically, the reaction is carried out using an activating agent, e.g. diethyl azodicarboxylate (DEAD) or diisopropyl azodicarboxylate (DIAD), and a phosphine reagent, e.g. triphenylphosphine or tributylphosphine, in an inert solvent, e.g. THF, dichloromethane, toluene or DMF, at a temperature between 0° C. and the boiling point of the solvent employed.

Further compounds of the invention can optionally also be prepared by conversions of functional groups of individual substituents, especially those listed for R³, proceeding from the compounds of the formula (I) obtained by above processes. These conversions are performed by customary methods known to those skilled in the art and include, for example, reactions such as nucleophilic and electrophilic substitutions, oxidations, reductions, hydrogenations, transition metal-catalysed coupling reactions, eliminations, alkylation, amination, esterification, ester hydrolysis, etherification, ether hydrolysis, formation of carbonamides, and introduction and removal of temporary protective groups.

The compounds of the invention have valuable pharmacological properties and can be used for prevention and treatment of diseases in humans and animals. The compounds of the invention offer a further treatment alternative and thus enlarge the field of pharmacy.

The compounds of the invention bring about vasorelaxation and inhibition of platelet aggregation, and lead to a decrease in blood pressure and to a rise in coronary blood flow. These effects are mediated by a direct stimulation of soluble guanylate cyclase and an intracellular rise in cGMP. In addition, the compounds of the invention enhance the action of substances which increase the cGMP level, for example EDRF (endothelium-derived relaxing factor), NO donors, protoporphyrin IX, arachidonic acid or phenylhydrazine derivatives.

The compounds of the invention are suitable for treatment and/or prophylaxis of cardiovascular, pulmonary, thromboembolic and fibrotic disorders.

Accordingly, the compounds of the invention can be used in medicaments for the treatment and/or prophylaxis of cardiovascular disorders such as, for example, high blood pressure (hypertension), acute and chronic heart failure, coronary heart disease, stable and unstable angina pectoris, peripheral and cardiac vascular disorders, arrhythmias, atrial and ventricular arrhythmias and impaired conduction such as, for example, atrioventricular blocks degrees I-III (AB block I-III), supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for the treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation such as, for example, pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angiopiasties (PTA), transluminal coronary angiopiasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for the treatment and/or prophylaxis of erectile dysfunction and female sexual dysfunction.

In the context of the present invention, the term “heart failure” encompasses both acute and chronic forms of heart failure, and also more specific or related types of disease, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure and acute phases of worsening of existing chronic heart failure (worsening heart failure).

In addition, the compounds of the invention can also be used for the treatment and/or prophylaxis of arteriosclerosis, impaired lipid metabolism, hypolipoproteinaemias, dyslipidaemias, hypertriglyceridaemias, hyperlipidaemias, hypercholesterolaemias, abetelipoproteinaemia, sitosteroiaemia, xanthomatosis, Tangier disease, adiposity, obesity and of combined hyperlipidaemias and metabolic syndrome.

The compounds of the invention can also be used for the treatment and/or prophylaxis of primary and secondary Raynaud's phenomenon, microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing.

The compounds of the invention are furthermore suitable for treating urological disorders such as, for example, benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI) such as, for example, mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds of the invention are also suitable for the treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example giutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

In addition, the compounds of the invention are also suitable for the treatment and/or prophylaxis of asthmatic disorders, pulmonary arterial hypertension (PAH) and other forms of pulmonary hypertension (PH) including left-heart disease-, HIV-, sickle cell anaemia-, thromboembolism (CTEPH)-, sarcoidosis-, COPD- or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active ingredients for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinization, multiple sclerosis, thalamic degeneration, Creutzfeldt-Jakob dementia, HIV dementia, schizophrenia with dementia or Korsakoff's psychosis. They are also suitable for the treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

In addition, the compounds of the invention are also suitable for controlling cerebral blood flow and are effective agents for controlling migraines. They are also suitable for the prophylaxis and control of sequelae of cerebral infarct (Apoplexia cerebri) such as stroke, cerebral ischaemias and skull-brain trauma. The compounds of the invention can likewise be used for controlling states of pain and tinnitus.

In addition, the compounds of the invention have anti-inflammatory action and can therefore be used as anti-inflammatory agents for the treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds of the invention can also be used for the treatment and/or prophylaxis of autoimmune diseases.

The compounds of the invention are also suitable for the treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds of the invention are also suitable for controlling postoperative scarring, for example as a result of glaucoma operations.

The compounds of the invention can also be used cosmetically for ageing and keratinized skin.

Moreover, the compounds of the invention are suitable for treatment and/or prophylaxis of hepatitis, neoplasms, osteoporosis, glaucoma and gastroparesis.

The present invention further provides for the use of the compounds of the invention for treatment and/or prophylaxis of disorders, especially the disorders mentioned above.

The present invention further provides for the use of the compounds of the invention for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides the compounds of the invention for use in a method for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides for the use of the compounds of the invention for production of a medicament for treatment and/or prophylaxis of disorders, especially the aforementioned disorders.

The present invention further provides for the use of the compounds of the invention for production of a medicament for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides a method for the treatment and/or prophylaxis of disorders, in particular the disorders mentioned above, using an effective amount of at least one of the compounds of the invention.

The present invention further provides a method for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis using an effective amount of at least one of the compounds of the invention.

The compounds of the invention can be used alone or, if required, in combination with other active compounds. The present invention further provides medicaments comprising at least one of the compounds of the invention and one or more further active compounds, especially for the treatment and/or prophylaxis of the aforementioned disorders. Preferred examples of active ingredients suitable for combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active compounds, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active compounds altering lipid metabolism, for example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein (a) antagonists.

Antithrombotic agents are preferably understood to mean compounds from the group of the platelet aggregation inhibitors, the anticoagulants or the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, dabigatran, melagatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an alpha-1-receptor blocker, by way of example and with preference prazosin.

In a preferred embodiment of the invention, the compounds of the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin AII antagonist, by way of example and with preference losartan, candesartan, valsartan, telmisartan or embursatan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with a mineralocorticoid receptor antagonist, by way of example and with preference spironolactone or eplerenone.

In a preferred embodiment of the invention, the compounds of the invention are administered in combination with a loop diuretic, for example furosemide, torasemide, bumetanide and piretanide, with potassium-sparing diuretics, for example amiloride and triamterene, with aidosterone antagonists, for example spironolactone, potassium canrenoate and epierenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein (a) antagonists.

In a preferred embodiment of the invention, the compounds of the invention are administered in combination with a CETP inhibitor, by way of example and with preference dalcetrapib, BAY 60-5521, anacetrapib or CETP vaccine (CETi-1).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds of the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorber, by way of example and with preference cholestyramine, colestipol, colesolvam, CholestaGel or colestimnide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein (a) antagonist, by way of example and with preference gemcabene calcium (CI-1027) or nicotinic acid.

The present invention further provides medicaments which comprise at least one compound according to the invention, typically together with one or more inert, non-toxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds of the invention can be converted to the administration forms mentioned. This can be accomplished in a manner known per se by mixing with inert, non-toxic, pharmaceutically suitable auxiliaries. These auxiliaries include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary in some cases to deviate from the stated amounts, specifically as a function of body weight, route of administration, individual response to the active compound, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight, Solvent ratios, dilution ratios and concentration data for the liquid/liquid solutions are based in each case on volume.

### A. EXAMPLES

### Abbreviations and Acronyms

aq. aqueous solution
calc. calculated
br. broad signal (NMR coup ing pattern)
CAS No. Chemical Abstracts Service number
δ shift in the NMR spectrum (stated in)
d doublet (NMR coupling pattern)
TLC thin-layer chromatography
DCI direct chemical ionization (in MS)
DMAP 4-N,N-dimethylaminopyridine
DMF dimethylformamide
DMSO dimethyl sulphoxide
EDCI N-[3-(dimethylamino)propyl]-N′-ethylcarbodiimide
eq. equivalent(s)
ESI electrospray ionization (in MS)
Et ethyl
ent enantiomerically pure
h hour(s)
HATU N-[(dimethylamino)(3H-[1,2,3]triazolo[4,5-b]-pyridin-3-yloxy)methylene]-N-methylmethanaminium hexafluorophosphate
HOBT 1H-benzotriazol-1-ol
HPLC high-pressure, high-performance liquid chromatography
HRMS high-resolution mass spectrometry
conc. concentrated
LC-MS liquid chromatography-coupled mass spectrometry
LiHMDS lithium hexamethyldisilazide
m multipet
Me methyl
min minute(s)
MS mass spectrometry
NMR nuclear magnetic resonance spectrometry
Pd₂dba₃ tris(dibenzylideneacetone)dipalladium
Ph phenyl
q quartet (NMR coupling pattern)
quint. quintet (NMR coupling pattern)
rac racemic
R_{F} retention factor (in thin-layer chromatography)
RuPhos 2-dicyciohexylphosphino-2′,6′-diisopropoxybiphenyl
RT room temperature
Rₜ retention time (in HPLC)
s singlet (NMR coupling pattern)
t triplet (NMR coupling pattern)
TFA trifluoroacetate
THF tetrahydrofuran
TBTU (benzotriazol-1-yloxy)bisdimethylaminomethylium fluoroborate
UV ultraviolet spectrometry
v/v ratio by volume (of a solution)
Xantphos 4,5-bis(diphenylphosphino)-9,9-dimethylxanthene
XPHOS dicyclohexyl(2′,4′,6-triisopropylbiphenyl-2-yl)phosphine

### LC/MS and HPLC Methods:

### LC-MS Methods (Analytical):

### Method A:

MS instrument type: Waters ZMD; HPLC instrument type: Waters 1525; column: Phenomenex Luna 3μ C18(2) 30 mm×4.6 mm; mobile phase A: water 0.1% formic acid, mobile phase B: acetonitrile 0.1% formic acid; gradient: 0.0 min 95% A->0.5 min 95% A->4.5 min 5% A->5.5 min 5/% A; flow rate: 2 ml/min; UV detection: DAD.

### Method B:

MS instrument type: Waters Micromass ZQ2000; HPLC instrument type: Waters Acquity UPLC system; column: Acquity UPLC BEH C18 1.7 Mikron 100 mm×2.1 mm; mobile phase A: water 0.1% formic acid, mobile phase B: acetonitrile 0.1% formic acid; gradient: 0.0 min 95% A->0.4 min 95% A->6.0 min 5% A->6.8 min 5% A; flow rate: 0.4 ml/min; UV detection: PDA.

### Method C:

MS instrument type: Waters ZQ; HPLC instrument type: HP1100 series; column: Phenomenex Luna 3μ C18(2) 30 mm×4.6 mm; mobile phase A: water 0.1% formic acid, mobile phase B: acetonitrile 0.1% formic acid; gradient: 0.0 min 95% A->0.5 min 95% A->4.5 min 5% A->5.5 min 5% A; flow rate: 2 ml/min; UV detection: PDA.

### Method D:

Instrument: Waters ACQUITY SQD UPLC system; column: Waters Acquity UPLC HSS T3 1.8μ 50×1 mm; mobile phase A: 1 l of water+0.25 ml of 99% strength formic acid; mobile phase B: 1 l of acetonitrile+0.25 ml of 99% strength formic acid; gradient: 0.0 min 90% A→1.2 min 5% A→2.0 min 5% A oven: 50° C.; flow rate: 0.40 ml/min; UV detection: 210-400 nm.

### Method E:

Instrument: Micromass Quattro Premier with Waters UPLC Acquity; column: Thermo Hypersil GOLD 1.9μ 50×1 mm; mobile phase A: 1 l of water+0.5 ml of 50% strength formic acid; mobile phase B: 1 l of acetonitrile+0.5 ml of 50% strength formic acid; gradient: 0.0 min 90% A→0.1 min 90% A→1.5 min 10% A→2.2 min 10% A; oven: 50° C.; flow rate: 0.33 ml/min; UV detection: 210 nm.

### LC-MS Methods (Preparative):

### Method F:

MS instrument type: Agilent 1260 Infinity purification system. Agilent 6100 series single quadrupol LC/MS; column: XSEELECT CSH Prep C18 5 μm OBD, 30×150 mm; mobile phase A: 0.1% strength aqueous formic acid, mobile phase B: 0.1% formic acid in acetonitrile; gradient: 10%-95%, 22 min, centred around a special focussed gradient; flow rate: 60 ml/min. Sample: injection of a 20-60 mg/ml solution in DMSO (+ optionally formic acid and water).

### Method G

MS instrument type: Agilent 1260 Infinity purification system. Agilent 6100 series single quadrupol LC/MS; column: XBridge Prep C18 5 μm OBD, 30×150 mm; mobile phase A: 0.1% aqueous ammonia, mobile phase B: 0.1% ammonia in acetonitrile; gradient: 10%-95%, 22 min, centred around a special focussed gradient; flow rate: 60 ml/min. Sample: injection of a 20-60 mg/ml solution in DMSO (+ optionally formic acid and water),

### Method H (GC-MS.)

Instrument: Thermo Scientific DSQII, Thermo Scientific Trace CC Ultra; column: Restek RTX-35MS, 15 m×200 μm×0.33 μm; constant flow rate with helium: 1.20 ml/min; oven: 60° C.; inlet: 220° C.; gradient: 60° C., 30° C./min→300° C. (maintained for 3.33 min).

### Method I (LC-MS);

Instrument: Waters ACQUITY SQD UPLC system; column: Waters Acquity UPLC HSS T3 1.8μ 30×2 mm; mobile phase A: 1 l of water+0.25 ml of 99% formic acid, mobile phase B: 1 l of acetonitrile+0.25 ml of 99% strength formic acid; gradient: 0.0 min 90% A→1.2 min 5% A→2.0 min 5% A oven: 50° C.; flow rate: 0.60 ml/min; UV detection: 208-400 nm.

### Method J:

Instrument: Thermo Fisher-Scientific DSQ; chemical ionization; reactant gas NH3; source temperature: 200° C.; ionization energy 70 eV.

### Method K (LC-MS):

MS instrument: Waters (Micromass) QM; HPLC instrument: Agilent 1100 series; column: Agilent ZORBAX Extend-C18 3.0×50 mm 3.5 micron; mobile phase A: 1 l of water+0.01 mol of ammonium carbonate, mobile phase B: 1 l of acetonitrile; gradient: 0.0 mini 98% A→0.2 min 98% A→3.0 min 5% A→4.5 min 5% A; oven: 40° C.; flow rate: 1.75 ml/min; UV detection: 210 nm.

### Method L (LC-MS):

Instrument: Micromass Quattro Premier with Waters UPLC Acquity; column: Thermo Hypersil GOLD 1.9μ 50×1 mm; mobile phase A: 1 l of water+0.5 ml of 50% formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

### Method M (LC-MS, Analytical):

MS instrument: Waters (Micromass) Quattro Micro; instrument Waters UPLC Acquity; column: Waters BEH C18 1.7μ 50×2.1 mm; mobile phase A: 1 l of water+0.01 mol of ammonium formate, mobile phase B: 1 l of acetonitrile; gradient: 0.0 min 95% A→0.1 mm 95% 95% A→2.0 min 15% A→2.5 min 15% A→2.51 min 10% A→3.0 min 10% A; oven: 40° C.; flow rate: 0.5 ml/min; UV detection: 210 nm.

### Method N (LC-MS, Analytical):

Instrument: Agilent MS Quad 6150; HPLC: Agilent 1290; column: Waters Acquity UPLC HSS T3 1.8μ 50×2.1 mm; mobile phase A: 1 l of water+0.25 ml of 99% formic acid, mobile phase B: 1 l of acetonitrile+0.25 ml of 99% strength formic acid; gradient: 0.0 min 90% A→0.3 min 90% A→1.7 min 5% A→3.0 min 5% A; oven: 50° C.; flow rate: 1.20 ml/min; UV detection: 205-305 nm.

### Method O (LC/MS, Analytical):

MS instrument type: Thermo Scientific FT-MS; UHPLC+ instrument type: Thermo Scientific UltiMate 3000; column: Waters, HSST3, 2.1×75 mm, C18 1.8 μm; mobile phase A: 1 l of water+0.01% formic acid; mobile phase B: 1 l of acetonitrile+0.01% formic acid; gradient: 0.0 min 10% B→2.5 min 95% B→3.5 min 95% B; oven: 50° C.; flow rate: 0.90 ml/min; UV detection: 210 nm/Optimum Integration Path 210-300 nm

### Method P (LC/MS, Analytical):

MS instrument type: HP 6130 MSD; HPLC instrument type: Agilent 1290 series; UV DAD; column: Waters Acquity HSS T3 1.8 μm 2.1 mm×75 mm; mobile phase A: ammonium acetate (10 mM)+water/methanol/acetonitrile (9.0:0.6:0.4), mobile phase B: ammonium acetate (10 mM)+water/methanol/acetonitrile (1.0:5.4:3.6), gradient: A/B: 80/20 (0.0 min)→(1.5 min)→0/100 (1.5 min); flow rate: 0.6 ml/min; oven: 35° C.; UV detection: 215 and 238 nm.

The multiplicities of proton signals in ¹H NMR spectra reported in the paragraphs which follow represent the signal form observed in each case and do not take account of any higher-order signal phenomena. In all ¹H NMR spectra data, the chemical shifts δ are stated in ppm.

Additionally, the starting materials, intermediates and working examples may be present as hydrates. There was no quantitative determination of the water content. In certain cases, the hydrates may affect the ¹H NMR spectrum and possibly shift and/or significantly broaden the water signal in the ¹H NMR.

Unless stated otherwise, the percentages in the tests and examples which follow are percentages by weight; parts are parts by weight. Solvent ratios, dilution ratios and concentration data for the liquid/liquid solutions are based in each case on volume.

The multiplicities of proton signals in ¹H NMR spectra reported in the paragraphs which follow represent the signal form observed in each case and do not take account of any higher-order signal phenomena. In all ¹H NMR spectra data, the chemical shifts δ are stated in ppm.

When compounds of the invention are purified by preparative HPLC by the above-described methods in which the mobile phases contain additives, for example trifluoroacetic acid, formic acid or ammonia, the compounds of the invention may be obtained in salt form, for example as trifluoroacetate, formate or ammonium salt, if the compounds of the invention contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### STARTING MATERIALS AND INTERMEDIATES

### Example 1A

### 3-[(2,6-Difluorobenzyl)oxy]pyridine-2-amine

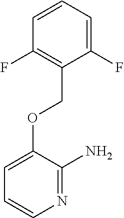 [SMILES: Nc1ncccc1OCc1c(F)cccc1F]

At room temperature, 51 g (953 mmol, 1.05 equivalents) of sodium methoxide were dissolved in 1000 ml of methanol, 100 g (908 mmol, 1 equivalent) of 2-amino-3-hydroxypyridine were added and the mixture was stirred at room temperature for a further 15 min. The reaction mixture was concentrated under reduced pressure, the residue was taken up in 2500 ml of dimethyl sulphoxide, and 197 g of cyclohexylmethyl bromide (953 mmol, 1.05 equivalents) of 2,6-difluorobenzyl bromide were added. After 4 h at room temperature, the reaction mixture was poured into 20 l of water and stirred for 15 min, and the solid was filtered off. The solid was washed with 1 l of water, 100 ml of isopropanol and 500 ml of petroleum ether and dried under high vacuum. This gave 171 g of the title compound (78% of theory).

¹H NMR (400 MHz, DMSO-d₆): δ=5.10 (s, 2H); 5.52 (br, s, 2H), 6.52 (dd, 1H); 7.16-7.21 (m, 3H); 7.49-7.56 (m, 2H).

### Example 2A

### 5-Bromo-3-[(2,6-di fluorobenzyl)oxy]pyridine-2-amine

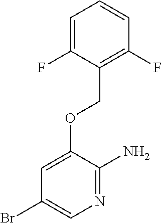 [SMILES: Nc1ncc(Br)cc1OCc1c(F)cccc1F]

32.6 g (138 mmol, 1 equivalent) of 3-[(2,6-difluorobenzyl)oxy]pyridine-2-amine (Example 1A) were suspended in 552 ml of 10% strength aqueous sulphuric acid, and the mixture was cooled to 0° C. 8.5 ml (165 mmol, 1.2 equivalents) of bromine were dissolved in 85 ml of acetic acid and then, over the course of 90 min, added dropwise to the ice-cooled reaction solution. The mixture was stirred at 0° C. for a further 90 min and the content was then diluted with 600 ml of ethyl acetate, and the aqueous phase was separated off. The aqueous phase was extracted with ethyl acetate. The organic phases were combined, washed with saturated aqueous sodium bicarbonate solution, dried and concentrated. The residue was dissolved in dichloromethane and chromatographed on silica gel (petroleum ether/ethyl acetate gradient as mobile phase). This gave 24 g (55% of theory) of the title compound.

LC-MS (Method D): Rₜ=0.96 min

MS (ESpos): m/z=315.1/317.1 (M+H)⁺

¹H NMR (400 MHz, DMSO-d₆): δ=5.14 (s, 2H); 5.83 (br. s, 2H); 7.20 (t, 2H); 7.42 (d, 1H); 7.54 (q, 1H); 7.62 (d, 1H).

### Example 3A

### Ethyl 6-bromo-8-[(2,6-difluorobenzyl)oxy]-2-methylimidazo[1,2-a]pyridine-3-carboxylate

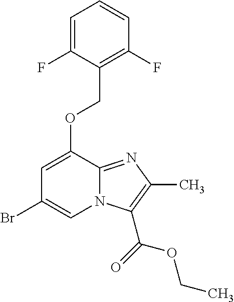 [SMILES: CCOC(=O)c1c(C)nc2c(OCc3c(F)cccc3F)cc(Br)cn12]

16 g of powdered molecular sieve 3 Å and 52.7 ml (380.8 mmol; 5 equivalents) of ethyl 2-chloroacetoacetate were added to 24 g (76.2 mmol; I equivalent) of 5-bromo-3-[(2,6-difluorobenzyl)oxy]pyridine-2-amine (Example 2A) in 400 ml of ethanol, and the mixture was heated at reflux overnight. A further 8 g of molecular sieve were added, and the mixture was heated at reflux for a further 24 h. The reaction mixture was cooled and concentrated under reduced pressure, and the residue was taken up in dichloromethane and chromatographed on silica gel (dichloromethane/methanol 20:1 as mobile phase). The product-containing fractions were concentrated and the residue was stirred in 100 ml of diethyl ether for 30 min. The product was filtered off, washed with a little diethyl ether and dried. This gave 15 g (45% of theory) of the title compound.

LC-MS (Method E): Rₜ=1.43 min

MS (ESpos): m/z=414.9/416.8 (M+H)⁺

¹H NMR (400 MHz, DMSO-d₆): δ=1.36 (t, 3H); 2.54 (s, 3H; hidden by the dimethyl sulfoxide signal); 4.37 (q, 2H); 5.36 (s, 2H); 7.25 (t, 2H); 7.42 (d, 1H); 7.61 (q, 1H); 9.00 (d, 1H).

### Example 4A

### 6-Bromo-8-[(2,6-difluorobenzyl)oxy]-2-methylimidazo[1,2-a]pyridine-3-carboxylic acid

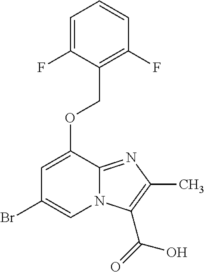 [SMILES: Cc1nc2c(OCc3c(F)cccc3F)cc(Br)cn2c1C(=O)O]

12.0 ml (12.0 mmol) of a 1 M aqueous sodium hydroxide solution were added to a solution of 5.0 g (11.8 mmol) of ethyl 6-bromo-8-[(2,6-difluorobenzyl)oxy]-2-methylimidazo[1,2-a]pyridine-3-carboxylate (Example 3A) in 30 ml of ethanol and 70 ml of tetrahydrofuran. The mixture was heated at reflux and stirred for 18 h. The mixture was then concentrated under reduced pressure and the residue was partitioned between water and ethyl acetate. The aqueous phase was separated off, and 1M aqueous hydrochloric acid was added until a pH of 3 had been reached. The aqueous mixture obtained was filtered, and the precipitate was washed with ethyl acetate and dried under high vacuum. This gave 4.7 g of the target product (100% of theory).

LC-MS (Method A): Rₜ=2.94 and 3.02 min; nm/z=397.399 (M+H)⁺

### Example 5A

### rac-tert-Butyl {1-[({6-bromo-8-[(2,6-difluorobenzyl)oxy]-2-methylmidazo[1,2-a]pyridin-3-yl}carbonyl)amino]-2-methylbutan-2-yl}carbamate

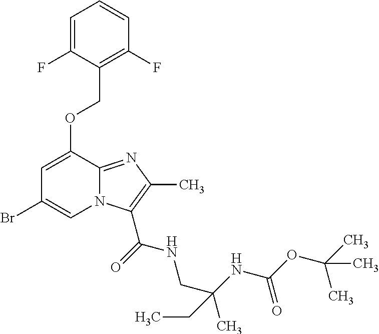 [SMILES: CCC(C)(CNC(=O)c1c(C)nc2c(OCc3c(F)cccc3F)cc(Br)cn12)NC(=O)OC(C)(C)C]

870 mg (6.4 mmol) of 1-hydroxy-7-azabenzotriazole and 1.22 g (6.4 mmol) of 1-ethyl-3-(3-dimethylaminopropyl)carbodiimide were added to a solution of 2.1 g (5.3 mmol) of 6-bromo-8-[(2,6-difluorobenzyl)oxy]-2-methylimidazo[1,2-a]pyridine-3-carboxylic acid (Example 4A), 2.7 ml (15.7 mmol) of diisopropylethylamine and 1.28 g (6.4 mmol) of tert-butyl rac-(1-amino-2-methylbutan-2-yl)carbamate (Example 18A) in 30 ml of tetrahydrofuran. The mixture was stirred at room temperature for 18 h and then concentrated under reduced pressure. The residue was partitioned between ethyl acetate and water. The organic phase was removed and washed with water and saturated aqueous sodium chloride solution, dried over sodium sulphate, filtered and concentrated. The residue was purified by chromatography on silica gel (120 g silica gel cartridge, mobile phase: cyclohexane/ethyl acetate, gradient 0% to 100%). This gave 2.9 g of the target product (94% of theory).

LC-MS (Method A): Rₜ=4.14 min; m/z=581, 583 (M+H)⁺

### Example 6A

### rac-tert-Butyl {1-[({8-[(2,6-difluorobenzyl)oxy]-2-methyl-6-(pyridin-3-yl)imidazo[1,2-a]pyridin-3-yl}carbonyl)amino]-2-methylbutan-2-yl}carbamate

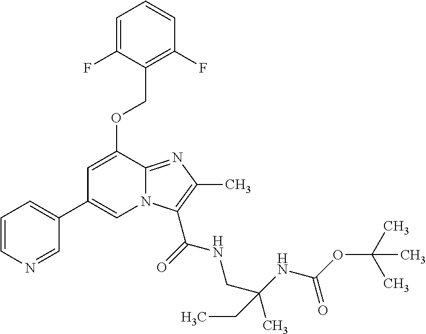 [SMILES: CCC(C)(CNC(=O)c1c(C)nc2c(OCc3c(F)cccc3F)cc(-c3cccnc3)cn12)NC(=O)OC(C)(C)C]

A mixture of 100 mg (0.17 mmol) of rac-tert-butyl {1-[({6-bromo-8-[(2,6-difluorobenzyl)oxy]-2-methylimidazo[1,2-a]pyridin-3-yl}carbonyl)amino]-2-methylbutan-2-yl}carbamate (Example 5A), 42 mg (0.21 mmol) of 3-(4,4,5,5-tetramethyl-[1,3,2]dioxaborolan-2-yl)pyridine, 14 mg (0.017 mmol) of 1,1′-bis(diphenylphosphino)ferrocenepalladium(II) dichloride/dichloromethane complex and 166 mg (0.51 mmol) of caesium carbonate in 0.5 ml of water and 2 ml of dioxane was degassed with argon for 5 min and stirred in a closed tube at 100° C. for 2 h. The reaction mixture was cooled to room temperature and the residue was partitioned between ethyl acetate and water. The organic phase was separated off, washed with saturated aqueous sodium chloride solution, dried over sodium sulphate, filtered and concentrated. The residue was purified by chromatography on silica gel (mobile phase: cyclohexane/ethyl acetate, gradient 0% to 50%). This gave 90 mg of the target product (90% of theory).

LC-MS (Method C): Rₜ=3.11 min; m/z=580 (M H)⁺

¹H-NMR (400 MHz, CDCl₃): δ [ppm]=0.95 (t, 3H), 1.24 (s, 3H), 1.42 (s, 9H), 1.61 (dd, 1H), 1.69 (s, 1H), 1.83 (dd, 1H), 2.77 (s, 3H), 3.76 (ddd, 2H), 4.58 (s, 1H), 5.44 (s, 2H), 6.95 (t, 2H), 7.04 (d, 1H), 7.31-7.40 (m, 2H), 7.92 (ddd, 1H), 8.63 (dd, 1H), 8.87 (dd, 1H), 9.33 (d, 1H).

### Example 7A

### rac-tert-Butyl {1-[({6-cyclopropyl-8-[(2,6-difluorobenzyl)oxy]-2-methylimidazo [12-a]pyridin-3-yl}carbonyl)amino]-2-methylbutan-2-yl}carbamate

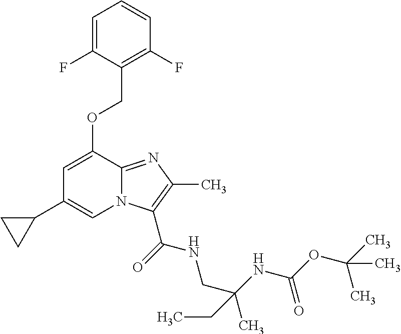 [SMILES: CCC(C)(CNC(=O)c1c(C)nc2c(OCc3c(F)cccc3F)cc(C3CC3)cn12)NC(=O)OC(C)(C)C]

A mixture of 100 mg (0.17 mmol) of rac-tert-butyl {1-[({6-bromo-8-[(2,6-difluorobenzyl)oxy]-2-methylimidazo[1,2-a]pyridin-3-yl}carbonyl)amino]-2-methylbutan-2-yl}carbamate (Example 5A), 38 μl (0.21 mmol) of 2-cyclopropyl-4,4,5,5-tetramethyl-[1,3,2]dioxaborolane, 14 mg (0.017 mmol) of 1,1′-bis(diphenylphosphino)ferrocenepalladium(II) dichloride/dichloromethane complex and 166 mg (0.51 mmol) of caesium carbonate in 0.5 ml of water and 2 ml of dioxane was degassed with argon for 5 min and stirred in a closed tube at 100° C. for 2 h. The reaction mixture was cooled to room temperature and the residue was partitioned between ethyl acetate and water. The organic phase was separated off, washed with saturated aqueous sodium chloride solution, dried over sodium sulphate, filtered and concentrated. The residue was purified by chromatography on silica gel (mobile phase: cyclohexane/ethyl acetate, gradient 0% to 50%). This gave 56 mg of the target product (60% of theory).

LC-MS (Method C): Rₜ=3.22 min, m/z=543 (M+H)⁺

¹H-NMR (400 MHz, CDCl₃): δ [ppm]=0.70-0.74 (m, 2H), 0.92-0.97 (m, 5H), 1.24 (s, 3H), 1.42 (s, 9H), 1.56-1.65 (m, 1H), 1.81 (td, 1H), 1.89-1.95 (m, 1H), 2.70 (s, 3H), 3.71 (dd, 1H), 3.78 (dd, 1H), 4.57 (s, 1H), 5.32 (s, 2H), 6.56 (d, 1H), 6.89-6.96 (m, 2H), 7.08 (s, 1H), 7.29-7.37 (m, 1H), 8.87 (s, 1H).

### Example 8A

### rac-tert-Butyl {1-[({8-[(2,6-difluorobenzyl)oxy]-2-methyl-6-(1H-pyrazol-1-yl)imidazo [1,2-a]pyridin-3-yl}carbonyl)amino]-2-methylbutan-2-yl}carbamate

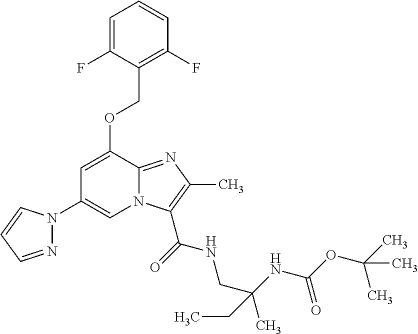 [SMILES: CCC(C)(CNC(=O)c1c(C)nc2c(OCc3c(F)cccc3F)cc(-n3cccn3)cn12)NC(=O)OC(C)(C)C]

A mixture of 100 mg (0.17 mmol) of rac-ter-butyl {1-[({6-bromo-8-[(2,6-difluorobenzyl)oxy]-2-methylimidazo[1,2-a]pyridin-3-yl}carbonyl)amino]-2-methylbutan-2-yl}carbamate (Example 5A), 18 mg (0.26 mmol) of 1H-pyrazole, 1.3 mg (0.009 mmol) of copper(I) oxide, 4.7 mg (0.034 mmol) of 2-hydroxybenzaldehyde oxime and 112 mg (0.34 mmol) caesium carbonate in 1 ml of acetonitrile was degassed with argon for 5 min and stirred in a closed tube at 82° C. for 18 h. The reaction mixture was concentrated and the residue was partitioned between dichloromethane and water. The organic phase was removed and concentrated. The residue was purified by chromatography on silica gel (mobile phase: cyclohexane/ethyl acetate, gradient 0% to 100%). This gave 15 mg of the target product Example 8A (13% of theory).

LC-MS (Method A): Rₜ=3.76 min, m/z=569 (M+H)⁺

### Example 9A

### rac-tert-Butyl {1-[({8-[(2,6-difluorobenzyl)oxy]-6-(methoxymethyl)-2-methylimidazo[1,2-a]pyridin-3-yl}carbonyl)amino]-2-methylbutan-2-yl}carbamate

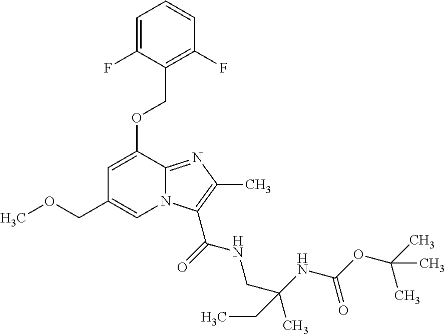 [SMILES: CCC(C)(CNC(=O)c1c(C)nc2c(OCc3c(F)cccc3F)cc(COC)cn12)NC(=O)OC(C)(C)C]

A mixture of 100 mg (0.17 mmol) of rac-tert-butyl {1-[(6-bromo-8-[(2,6-difluorobenzyl)oxy]-2-methylimidazo[1,2-a]pyridin-3-yl}carbonyl)amino]-2-methylbutan-2-yl carbamate (Example 5A), 29 mg (0.19 mmol) of potassium (methoxymethyl)trifluoroborate, 1.9 mg (0.008 mmol) of palladium(II) acetate, 8.0 mg (0.017 mmol) of RuPhos and 168 mg (0.52 mmol) of caesium carbonate in 0.1 ml of water and 1 ml of dioxane was degassed with argon for 5 min and stirred in a closed tube at 100° C. for 18 h. The reaction mixture was concentrated and the residue was partitioned between ethyl acetate and water. The organic phase was separated off, washed with saturated aqueous sodium chloride solution, dried over sodium sulphate, filtered and concentrated. The residue was purified by chromatography on silica gel (mobile phase: cyclohexane/ethyl acetate, gradient 0% to 50%). This gave 60 mg of the target product (64% of theory).

LC-MS (Method A): Rₜ=3.08 min; m/z=547.1 (M+H)⁺

¹H-NMR (400 MHz, CDCl₃): δ [ppm]=0.95 (t, 3H), 1.42 (s, 9H), 1.43 (s, 3H), 1.60 (dd, 1H), 1.66 (s, 1H), 1.81 (dd, 1H), 2.73 (s, 3H), 3.38 (s, 3H), 3.75 (ddd, 2H), 4.45 (s, 2H), 4.57 (s, 1H), 5.33 (s, 2H), 6.86 (d, 1H), 6.93 (dd, 2H), 7.29-7.38 (m, 1H), 9.03 (d, 1H).

### Example 10A

### rac-tert-Butyl {1-[({8-[(2,6-difluorobenzyl)oxy]-2-methyl-6-(4,4,5,5-tetramethyl-1,3,2-dioxaborolan-2-yl)imidazo[1,2-a]pyridin-3-yl}carbonyl)amino]-2-methylbutan-2-yl}carbamate

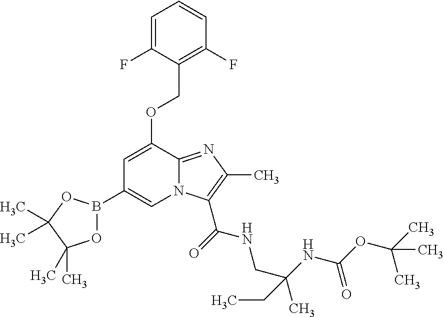 [SMILES: CCC(C)(CNC(=O)c1c(C)nc2c(OCc3c(F)cccc3F)cc(B3OC(C)(C)C(C)(C)O3)cn12)NC(=O)OC(C)(C)C]

A mixture of 434 mg (0.75 mmol) of rac-tert-butyl {1-[({6-bromo-8-[(2,6-difluorobenzyl)oxy]-2-methylimidazo[1,2-a]pyridin-3-yl}carbonyl)amino]-2-methylbutan-2-yl}carbamate (Example 5A), 228 mg (0.90 mmol) of bis(pinacolato)diboron, 30 mg (0.037 mmol) of 1,1-bis(diphenylphosphino)ferrocenepalladium(II) dichloride/dichloromethane complex and 220 mg (2.2 mmol) of potassium acetate in 4 ml of dioxane was degassed with argon for 5 min and stirred in a closed tube at 80° C. for 18 h. The reaction mixture was cooled to room temperature and the residue was partitioned between ethyl acetate and water. The organic phase was removed and washed with saturated aqueous sodium chloride solution, dried over sodium sulphate, filtered and concentrated. This gave 563 mg of crude target product.

LC-MS (Method A): Rₜ=2.72 min; m/z=547.1 (M+H)+

### Example 11A

### rac-tert-Butyl {-[({8-[(2,6-difluorobenzyl)oxy]-6-hydroxy-2-methylimidazo [1,2-a]pyridin-3-yl}carbonyl)amino]-2-methylbutan-2-yl}carbamate

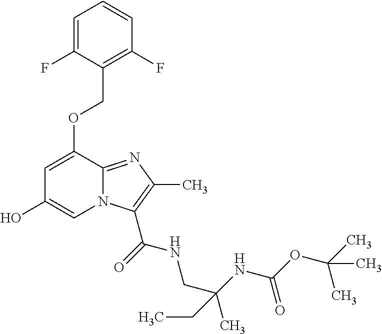 [SMILES: CCC(C)(CNC(=O)c1c(C)nc2c(OCc3c(F)cccc3F)cc(O)cn12)NC(=O)OC(C)(C)C]

A mixture of 100 ng (0.16 mmol) of rac-tert-butyl {1-[({8-[(2,6-difluorobenzyl)oxy]-2-methyl-6-(4,4,5,5-tetramethyl-1,3,2-dioxaborolan-2-yl)imidazo[1,2-a]pyridin-3-yl}carbonyl)amino]-2-methylbutan-2-yl}carbamate (Example 10A), 0.16 ml of 30% strength aqueous hydrogen peroxide and 1 ml of 1M aqueous sodium hydroxide solution in 2 ml of tetrahydrofuran was stirred at 0° C. for 30 min. The resulting mixture was partitioned between ethyl acetate and 1% strength aqueous citric acid. The organic phase was separated off, washed with saturated aqueous sodium chloride solution, dried over sodium sulphate, filtered and concentrated. The residue was purified by chromatography on silica gel (12 g silica gel cartridge, mobile phase: cyclohexane/ethyl acetate, gradient 0% to 100%). This gave 50 mg of the target product (60% of theory).

LC-MS (Method A): Rₜ=2.79 min; m/z=519 (M+H)⁺

### Example 12A

### rac-tert-Butyl {1-[({8-[(2,6-difluorobenzyl)oxy]-6-(difluoromethoxy)-2-methylimidazo [1,2-a]pyridin-3-yl}carbonyl)amino]-2-methylbutan-2-yl}carbamate

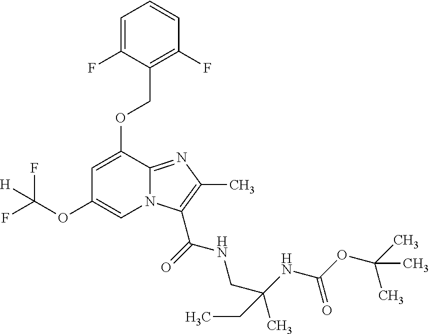 [SMILES: CCC(C)(CNC(=O)c1c(C)nc2c(OCc3c(F)cccc3F)cc(OC(F)F)cn12)NC(=O)OC(C)(C)C]

50 mg (0.10 mmol) of rac-tert-butyl {1-[({8-[(2,6-difluorobenzyl)oxy]-6-hydroxy-2-methylimidazo[1,2-a]pyridin-3-yl}carbonyl)amino]-2-methylbutan-2-yl}carbamate (Example 11A), 71 mg (0.47 mmol) of sodium chlorodifluoroacetate and 226 mg (0.69 mmol) of caesium carbonate in 1 ml of dimethylformamide were stirred at 80° C. for 2 h. The reaction mixture was cooled to room temperature and the residue was partitioned between ethyl acetate and water. The organic phase was separated off, washed with saturated aqueous sodium chloride solution, dried over sodium sulphate, filtered and concentrated. The residue was purified by chromatography on silica gel (12 g silica gel cartridge, mobile phase: ethyl acetate/cyclohexane, gradient 0% to 50%). This gave 13 mg of the target product (24% of theory).

LC-MS (Method A): Rₜ=3.86 min; m/z=569 (M+H)⁺

¹H-NMR (400 MHz, CDCl₃): δ [ppm]=0.95 (t, 3H), 1.24 (s, 3H), 1.42 (s, 9H), 1.60 (dd, 1H), 1.63 (s, 1H), 1.82 (dd, 1H), 2.73 (s, 3H), 3.73 (ddd, 2H), 4.56 (s, 1H), 5.33 (s, 2H), 6.52 (t, 1H), 6.75 (d, 1H), 6.94 (dd, 1H), 7.30-7.46 (m, 2H), 9.10 (d, 1H).

### Example 13A

### rac-tert-Butyl {1-[({8-[(2,6-difluorobenzyl)oxy]-2-methyl-6-(1,3-oxazol-5-yl)imidazo[1,2-a]pyridin-3-yl}carbonyl)amino]-2-methylbutan-2-yl}carbamate

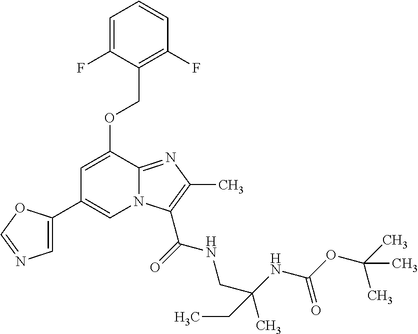 [SMILES: CCC(C)(CNC(=O)c1c(C)nc2c(OCc3c(F)cccc3F)cc(-c3cnco3)cn12)NC(=O)OC(C)(C)C]

A mixture of 100 mg (0.17 mmol) of rac-tert-butyl {1-[({6-bromo-8-[(2,6-difluorobenzyl)oxy]-2-methylimidazo[1,2-a]pyridin-3-yl}carbonyl)amino]-2-methylbutan-2-yl}carbamate (Example 5A), 40 mg (0.21 mmol) of 5-(4,4,5,5-tetramethyl-[1,3,2]dioxaborolan-2-yl)oxazole, 14 mg (0.017 mmol) of 1,1′-bis(diphenylphosphino)ferrocenepalladium(II) dichloride/dichloromethane complex and 166 mg (0.51 mmol) of caesium carbonate in 0.5 ml of water and 2 ml of dioxane was degassed with argon for 5 min and stirred in a closed tube at 100° C. for 2 h. The reaction mixture was cooled to room temperature and the residue was partitioned between ethyl acetate and water. The organic phase was separated off, washed with saturated aqueous sodium chloride solution, dried over sodium sulphate, filtered and concentrated. The residue was purified by chromatography on silica gel (mobile phase: cyclohexane/ethyl acetate, gradient 0% to 50%). This gave 43 mg of the target product (44% of theory).

LC-MS (Method A): Rₜ=3.54 min; m/z=570 (M+H)⁺

¹H-NMR (400 MHz, CDCl₃): δ [ppm]=0.95 (t, 3H), 1.25 (s, 3H), 1.42 (s, 9H), 1.55-1.67 (m, 2H), 1.78-1.88 (m, 1H), 2.75 (s, 3H), 3.76 (ddd, 21H), 4.58 (s, 1H), 5.43 (s, 21H), 6.95 (t, 2H), 7.04 (d, 1H), 7.30-7.40 (m, 2H), 7.92 (s, 1H), 9.46 (d, 1H).

### Example 14A

### Methyl 8-[(2, 6-difluorobenzyl)oxy]-2-(methoxymethyl)-6-methylimidazo [1,2-a]pyridine-3-carboxylate

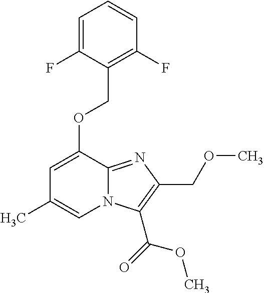

0.5 ml (2.87 mmol) of diisopropylethylamine were added to a solution of 390 mg (2.17 mmol) of methyl 2-chloro-4-methoxy-3-oxobutanoate and 450 mg (1.80 mmol) of 3-[(2,6-difluorobenzyl)oxy]-5-methylpyridine-2-amine in 5 ml of 1,2-dimethoxyethane, and the mixture was stirred at reflux for 18 h. The reaction mixture was concentrated and the residue was partitioned between ethyl acetate and an aqueous saturated solution of sodium bicarbonate. The organic phase was removed, washed with water and saturated aqueous sodium chloride solution, dried over sodium sulphate, filtered and concentrated. The residue was purified by chromatography on silica gel (40 g silica gel cartridge, mobile phase: cyclohexane/ethyl acetate, gradient 0% to 100%). This gave 280 mg of the target product (41% of theory).

LC-MS (Method A): Rₜ=2.46 min; m/z=377 (M+H)⁺

### Example 15A

### 8-[(2,6-Difluorobenzyl)oxy]-2-(methoxymethyl)-6-methylimidazo[1,2-a]pyridine-3-carboxylic acid

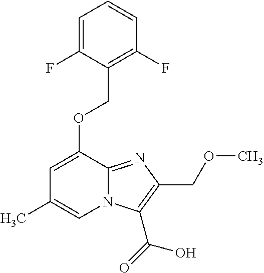

0.75 ml (0.75 mmol) of 1 M aqueous sodium hydroxide solution was added to a solution of 270 mg (0.745 mmol) of methyl 8-[(2,6-difluorobenzyl)oxy]-2-(methoxymethyl)-6-methylimidazo[1,2-a]pyridine-3-carboxylate (Example 14A) in 10 ml of methanol. The reaction mixture was stirred at room temperature for 18 h. 0.75 ml (0.75 mmol) of 1M aqueous sodium hydroxide solution was added to the mixture, and stirring was continued at room temperature for a further 5 h. The resulting mixture was cooled to 5° C. and neutralized by addition of 1.5 ml of a 1M aqueous hydrochloric acid solution. The resulting mixture was concentrated to dryness and the residue was distilled azeotropically with toluene, giving 350 mg of the target compound (100% of theory),

LC-MS (Method A): Rₜ=2.30 min; m/z=363 (M+H)⁺

### Example 16A

### rac-tert-Butyl {1-[({8-[(2,6-difluorobenzyl)oxy]-2-(methoxymethyl)-6-methylimidazo[1,2-a]pyridin-3-yl}carbonyl)amino]-2-methylbutan-2-yl}carbamate

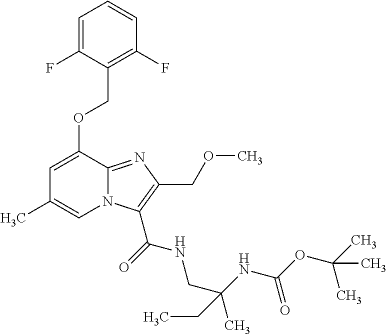 [SMILES: CCC(C)(CNC(=O)c1c(COC)nc2c(OCc3c(F)cccc3F)cc(C)cn12)NC(=O)OC(C)(C)C]

118 mg (0.87 mmol) of 1-hydroxy-7-azabenzotriazole and 166 mg (0.87 mmol) of 1-ethyl-3-(3-dimethylaminopropyl)carbodiimide were added to a solution of 261 mg (072 mmol) of 8-[(2,6-difluorobenzyl)oxy]-2-(methoxymethyl)-6-methylimidazo[1,2-a]pyridine-3-carboxylic acid (Example 15A), 372 μl (2.14 mmol) of diisopropylethylamine and 174 mg (0.86 mmol) of tert-butyl rac-(1-amino-2-methylbutan-2-yl)carbamate (Example 18A) in 10 ml of tetrahydrofuran. The mixture was stirred at room temperature for 2 days and concentrated under reduced pressure. The residue was partitioned between dichloromethane and water. The organic phase was removed, washed with water and saturated aqueous sodium chloride solution, dried over sodium sulphate, filtered and concentrated. The residue was purified by chromatography on silica gel (40 g silica gel cartridge, mobile phase: cyclohexane/ethyl acetate, gradient 0% to 100%), This gave 258 mg of the target product (66% of theory).

LC-MS (Method A): Rₜ=2.76 min; m/z=547 (M+H)⁺

¹H-NMR (400 MHz, CDCl₃): δ [ppm]=0.78 (dd, 3H), 1.11 (s, 3H), 1.28 (s, 9H), 1.59-1.48 (m, 1H), 1.72-1.63 (m, 1H), 2.33 (d, 3H), 3.26 (dd, 1H), 3.48 (dd, 1H), 3.73 (s, 2H), 3.97 (s, 3H), 4.70 (s, 1H), 5.29 (s, 2H), 6.46 (d, 1H), 6.94 (dd, 2H), 7.40-7.33 (m, 2H), 7.46 (s, 1H).

### Example 17A

### rac-tert-Butyl (2-cyanobutan-2-yl)carbamate

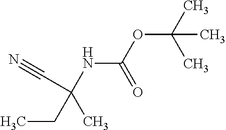

At room temperature (maximum temperature 30° C.), 30 g (305.7 mmol) of rac-2-amino-2-methylbutanenitrile were added slowly to 73.38 g (336.2 mmol) of di-tert-butyl dicarboxylate. The mixture was stirred at room temperature overnight. Dichloromethane was added and the mixture was washed twice with 1 N aqueous sodium hydroxide solution. The organic phase was separated off, dried over sodium sulphate and concentrated (maximum temperature 30° C.). This gave 44.2 g of the target product (73% of theory).

GC-MS (Method H): Rₜ: 4.04 min, m/z: 98 (M-Boc)⁺

### Example 18A

### rac-tert-Butyl (1-amino-2-methylbutan-2-yl)carbamate

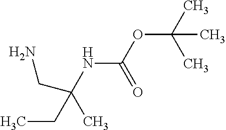

44.2 g (222.93 mmol) rac-tert-butyl (2-cyanobutan-2-yl)carbamate (Example 17A) were dissolved in 500 ml of a 7 M solution of ammonia in methanol, and 45 g of Raney nickel (50% suspension in water) were added. For 18 hours, the reaction mixture was kept in an autoclave at room temperature and a hydrogen pressure of 30 bar. The reaction mixture was filtered through a layer of Celite which was washed with methanol, and the combined filtrates were concentrated under reduced pressure (maximum temperature: 40° C.). This gave 45 g of the target product (100% of theory).

LC-MS (Method D): Rₜ=0.18 min

MS (ESpos): m/z=203 (M+H)⁺

### Example 19A

### 3-(Benzyloxy)-5-bromopyridine-2-amine

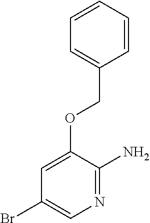 [SMILES: Nc1ncc(Br)cc1OCc1ccccc1]

200 g (1 mol) of 2-amino-3-benzyloxypyridine were initially charged in 4 l of dichloromethane, and at 0° C. a solution of 62 ml (1.2 mol) of bromine in 620 ml of dichloromethane was added over 30 min. After the addition had ended, the reaction solution was stirred at 0° C. for 60 min. About 4 l of saturated aqueous sodium bicarbonate solution were then added to the mixture. The organic phase was removed and concentrated. The residue was purified by silica gel column chromatography (petroleum ether/ethyl acetate 6:4) and the product fractions were concentrated. This gave 214 g (77% of theory) of the title compound.

LC-MS (Method D): Rₜ=0.92 min

MS (ESpos): m/z=279 (M+H)⁺

¹H-NMR (400 MHz, DMSO-d₆): δ=5.16 (s, 2H), 5.94-6.00 (min, 2H), 7.26-7.29 (m, 1H), 7.31-7.36 (m, 1H), 7.37-7.43 (m, 2H), 7.47-7.52 (m, 2H), 7.57-7.59 (m, 1H).

### Example 20A

### Ethyl 8-(benzyloxy)-6-bromo-2-methylimidazo[1,2-a]pyridine-3-carboxylate

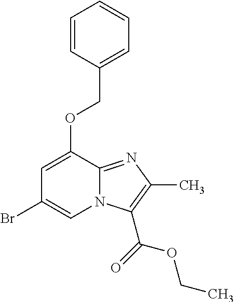 [SMILES: CCOC(=O)c1c(C)nc2c(OCc3ccccc3)cc(Br)cn12]

Under argon, 200 g (0.72 mol) of 3-(benzyloxy)-5-bromopyridine-2-amine from Example 19A, 590 g (3.58 mol) of ethyl 2-chloroacetoacetate and 436 g of 3 A molecular sieve were suspended in 6 l of ethanol, and the suspension was stirred at reflux for 72 h. The reaction mixture was filtered off through silica gel and concentrated. The residue was purified by silica gel chromatography (petroleum ether:ethyl acetate 9:1, then 6:4) and the product fractions were concentrated. This gave 221 g (79% of theory) of the target compound.

LC-MS (Method I): Rₜ=1.31 min

MS (ESpos): m/z=389 (M+H)⁺

¹H-NMR (400 MHz, DMSO-d₆): δ=1.36 (t, 3H), 2.58 (s, 3H), 4.32-4.41 (m, 2H), 5.33 (s, 2H), 7.28-7.32 (m, 1H), 7.36-7.47 (m, 3H), 7.49-7.54 (m, 2H), 8.98 (d, 1H).

### Example 21A

### Ethyl 8-(benzyloxy)-2,6-dimethylimidazo [1,2-a]pyridine-3-carboxylate

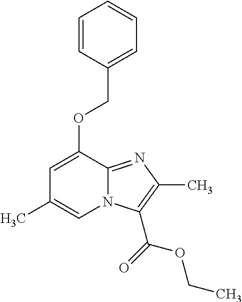 [SMILES: CCOC(=O)c1c(C)nc2c(OCc3ccccc3)cc(C)cn12]

Under argon, 105 g (270 mmol) of ethyl 8-(benzyloxy)-6-bromo-2-methylimidazo[1,2-a]pyridine-3-carboxylate from Example 20A were suspended in 4.2 l of 1,4-dioxane, and 135.4 g (539 mmol, purity 50%) of trimethylboroxine, 31.2 g (27 mmol) of tetrakis(triphenylphosphine)palladium(0) and 78.3 g (566 mmol) of potassium carbonate were added in succession and the mixture was stirred under reflux for 8 h. The precipitate of the reaction mixture, cooled to RT, was removed by filtration over silica gel, and the filtrate was concentrated. The residue was dissolved in dichloromethane and purified by silica gel chromatography (dichloromethane:ethyl acetate=9:1). This gave 74 g (84.6% of theory) of the target compound.

LC-MS (Method 1): Rₜ=1.06 min

MS (ESpos): m/z=325 (M+H)⁺

¹H-NMR (400 MHz, DMSO-d₆): δ=1.35 (t, 3H), 2.34 (br. s, 3H), 2.56 (s, 3H), 4.31-4.38 (m, 2H), 5.28 (br. s, 2H), 6.99-7.01 (m, 1H), 7.35-7.47 (m, 3H), 7.49-7.54 (m, 2H), 8.68-8.70 (m, 1H).

### Example 22A

### Ethyl 8-hydroxy-2,6-dimethylimidazo[1,2-a]pyridine-3-carboxylate

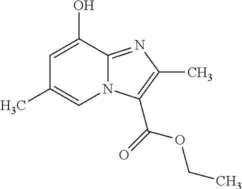 [SMILES: CCOC(=O)c1c(C)nc2c(O)cc(C)cn12]

74 g (228 mmol) of ethyl 8-(benzyloxy)-2,6-dimethylimidazo[1,2-a]pyridine-3-carboxylate from Example 21A were initially charged in 1254 ml of dichloromethane and 251 ml of ethanol, and 20.1 g of 10% palladium on activated carbon (moist with water, 50%) were added under argon. The reaction mixture was hydrogenated at RT and under standard pressure overnight. The reaction mixture was filtered off through silica gel and concentrated. The crude product was purified by silica gel chromatography (dichloromethane:methanol=95:5). This gave 50.4 g (94% of theory) of the target compound.

DCI-MS: (Method J) (ESpos): m/z=235.2 (M+H)⁺

¹H-NMR (400 MHz, DMSO-d₆): δ=1.35 (t, 3H), 2.27 (s, 3H), 2.58 (s, 3H), 4.30-4.38 (m, 2H), 6.65 (d, 1H), 8.59 (s, 1H), 10.57 (br. s, 11H).

### Example 23A

### Ethyl 8-[(2,6-difluorobenzyl)oxy]-2,6-dimethylimidazo[1,2-a]pyridine-3-carboxylate

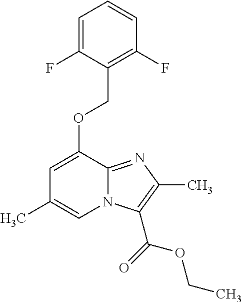 [SMILES: CCOC(=O)c1c(C)nc2c(OCc3c(F)cccc3F)cc(C)cn12]

20.00 g (85.38 mmol) of ethyl 8-hydroxy-2,6-dimethylimidazo[1,2-a]pyridine-3-carboxylate from Example 22A, 19.44 g (93.91 mmol) of 2,6-difluorobenzyl bromide and 61.20 g (187.83 mmol) of caesium carbonate in 1.18 l of DMF were stirred at 60° C. for 5 h. The reaction mixture was then poured into 6.4 l of 10% strength aqueous sodium chloride solution and then twice extracted with ethyl acetate. The combined organic phases were washed with 854 ml of a 10% strength aqueous sodium chloride solution, dried, concentrated and dried at RT under high vacuum overnight. This gave 28.2 g (92% of theory; purity about 90%) of the title compound.

LC-MS (Method D): Rₜ=1.05 min

MS (ESpos): m/z=361.1 (M+H)⁺

¹H-NMR (400 MHz, DMSO-d₆): δ=1.38 (t, 3H); 2.36 (s, 3H); 4.35 (q, 2H); 5.30 (s, 2H); 7.10 (s, 1H); 7.23 (t, 2H); 7.59 (q, 1H); 8.70 (s, 1H).

### Example 24A

### 8-[(2,6-Difluorobenzyl)oxy]-2,6-dimethylimidazo[1,2-a]pyridine-3-carboxylic acid

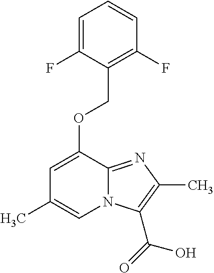

220 mg of ethyl 8-[(2,6-difluorobenzyl)oxy]-2,6-dimethylimidazo[1,2-a]pyridine-3-carboxylate (Example 23A; 0.524 mmol, 1 equivalent) were dissolved in 7 ml of THF/methanol (1:1), 2.6 ml of 1 N aqueous lithium hydroxide solution (2.6 mmol, 5 equivalents) were added and the mixture was stirred at RT for 16 h. The mixture was concentrated under reduced pressure and the residue was acidified with 1N aqueous hydrochloric acid and stirred for 15 min. The solid was filtered off, washed with water and dried under reduced pressure. This gave 120 mg of the title compound (60% of theory).

LC-MS (Method D): Rₜ=0.68 min

MS (ESpos): m/z=333.1 (M+H)⁺

¹H-NMR (400 MHz, DMSO-d₆): δ=2.34 (s, 3H); 5.28 (s, 2H); 7.09 (s, 1H); 7.23 (t, 2H); 7.58 (q, 1H); 8.76 (s, 1H); 13.1 (br. s, 1H), [further signal hidden under DMSO signal].

### Example 25A

### Ethyl 8-[(3-fluoropyridin-2-yl)methoxy]-2,6-dimethylimidazo[1,2-a]pyridine-3-carboxylate

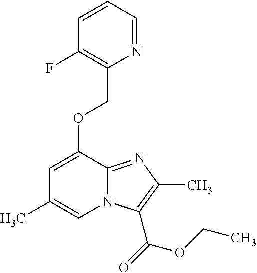 [SMILES: CCOC(=O)c1c(C)nc2c(OCc3ncccc3F)cc(C)cn12]

15.78 g (86.7 mmol) of 2-(chloromethyl)-3-fluoropyridine hydrochloride (commercially available, also described in: U.S. Pat. No. 5,593,993 A1, 1997; WO2007/2181 A2, 2007) and 94.06 g (288.9 mmol) of caesium carbonate were added to 16.92 g (72.2 mmol) of ethyl 8-hydroxy-2,6-dimethylimidazo[1,2-a]pyridine-3-carboxylate from Example 22A in 956 ml of DMF. The reaction mixture was stirred at 60° C. overnight. The reaction mixture, cooled to RT, was filtered, the filter cake was washed with ethyl acetate and the filtrate was concentrated. About 500 ml of water were added to the residue, and the precipitated solid was filtered off and dried under high vacuum. This gave 24.1 g (93% of theory) of the target compound.

LC-MS (Method D): Rₜ=0.84 min

MS (ESpos): m/z=344 (M+H)⁺

¹H-NMR (400 MHz, DMSO-d₆): δ=1.35 (t, 3H), 2.35 (s, 3H), 2.54 (s, 3H, hidden by the DMSO signal), 4.35 (q, 2H), 5.40 (s, 2H), 7.08 (s, 1H), 7.55-7.62 (m, 1H), 7.82-7.89 (m, 1H), 8.48-8.52 (m, 1H), 8.70 (s, 1H).

### Example 26A

### 8-[(3-Fluoropyridin-2-yl)methoxy]-2,6-dimethylimidazo[1,2-a]pyridine-3-carboxylic acid hydrochloride

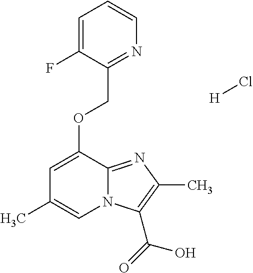

24.06 g (70.1 mmol) of ethyl 8-[(3-fluoropyridin-2-yl)methoxy]-2,6-dimethylimidazo[1,2-a]pyridine-3-carboxylate from Example 25A were initially charged in 1.5 l of THF/methanol (5:1), 350.4 ml (350.4 mmol) of 1 N aqueous lithium hydroxide solution were added and the reaction mixture was stirred at 40° C. for 2.5 h. After cooling, the pH was adjusted to about 4 using I N aqueous hydrochloric acid, and the solution was freed of THF/methanol under reduced pressure. The residue was cooled and the precipitated solid was filtered off and dried under reduced pressure. This gave 22.27 g (100% of theory) of the title compound.

LC-MS (Method D): Rₜ=0.55 min

MS (ESpos): m/z=316 (M+H)⁺

¹H-NMR (400 MHz, DMSO-d₆): δ=2.34 (s, 3H), 2.53 (s, 3H, hidden by the DMSO signal), 5.38-5.42 (m, 2H), 7.06 (s, 1H), 7.56-7.62 (m, 1H), 7.82-7.89 (m, 1H), 8.48-8.52 (m, 1H), 8.74 (s, 1H), 13.02 (br. s, 1H).

### Example 27A

### 5-Chloro-2-nitropyridin-3-ol

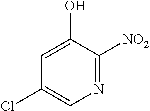 [SMILES: O=[N+]([O-])c1ncc(Cl)cc1O]

With ice cooling, 30 g of 5-chloropyridin-3-ol (232 mmol, I equivalent) were dissolved in 228 ml of concentrated sulphuric acid, and 24 ml of concentrated nitric acid were added slowly at 0° C. The reaction was warmed to RT and stirred overnight. The mixture was stirred into an ice/water mixture and stirred for 30 min. The solid was filtered off, washed with cold water and air-dried. This gave 33 g (82% of theory) of the title compound which was used without further purification for the next reaction.

LC-MS (Method D): Rₜ=0.60 min

MS (ESneg): m/z=172.9/174.9 (M−H)⁻

¹H-NMR (400 MHz, DMSO-d₆): δ=7.71 (d, 1H); 8.10 (d, 1H); 12.14 (br. 1H).

### Example 28A

### 5-Chloro-3-[(3-fluoropyridin-2-yl)methoxy]-2-nitropyridine

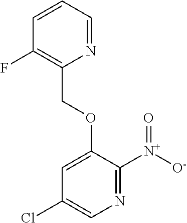 [SMILES: O=[N+]([O-])c1ncc(Cl)cc1OCc1ncccc1F]

20.0 g (114.6 mmol) of 5-chloro-2-nitropyridin-3-ol from Example 27A and 56.0 g (171.9 mmol) of caesium carbonate were initially charged in 319 ml of DMF. 17.51 g (120.3 mmol) of 2-(chloromethyl)-3-fluoropyridine (commercially available; additionally described in: K. Weidmann et al. Journal of Medicinal Chemistry 1992, 35, 438-450; U.S. Pat. No. 5,593,993, 1997; WO2007/2181 A2, 2007) were added and the reaction mixture was stirred at RT overnight. 6.0 g (41.2 mmol) of 2-(chloromethyl)-3-fluoropyridine were added and the mixture was stirred at RT for 24 h. Subsequently, another 6.0 g (41.2 mmol) of 2-(chloromethyl)-3-fluoropyridine and 5.0 g (15.3 mmol) of caesium carbonate were added and the mixture was stirred at 60° C. for 12 h. The reaction mixture was added carefully to 2.3 l of 0.5 M aqueous hydrochloric acid. The mixture was extracted three times with in each case 500 ml of ethyl acetate. The combined organic phases were washed with 500 ml of saturated aqueous sodium chloride solution, dried and concentrated under reduced pressure. The crude product was purified by means of silica gel chromatography (mobile phase: cyclohexane/ethyl acetate gradient: 9/1 to 7/3). This gave 29.8 g (92% of theory) of the target compound.

LC-MS (Method D): Rₜ=0.94 min.

MS (ESIpos): m/z=284 (M+H)⁺.

¹H-NMR (400 MHz, DMSO-d₆): δ=5.59 (d, 2H), 7.53-7.60 (m, 1H), 7.80-7.87 (m, 1H), 8.26 (d, 1H), 8.40-8.47 (m, 2H).

### Example 29A

### 5-Chloro-3-[(3-fluoropyridin-2-yl)methoxy]pyridine-2-amine

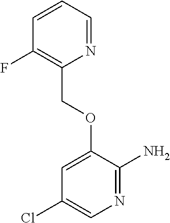 [SMILES: Nc1ncc(Cl)cc1OCc1ncccc1F]

Under argon, 29.8 g (105.1 mmol) of 5-chloro-3-[(3-fluoropyridin-2-yl)methoxy]-2-nitropyridine from Example 28A were initially charged in 317 ml of ethanol. 18.2 g (325.7 mmol) of iron powder were added, and the reaction mixture was heated to reflux. 80.4 ml of conc. hydrochloric acid were slowly added dropwise and the mixture was heated under reflux for a further 6 h. The reaction mixture was made alkaline with 33% strength ammonia solution and then concentrated under reduced pressure. Purification by silica gel chromatography (mobile phase: dichloromethane/methanol gradient 95/5 to 90/10) gave 25.0 g (94% of theory) of the target compound.

LC-MS (Method D): Rₜ=0.70 min

MS (ESIpos): m/z=254 (M+H)⁺

¹H-NMR (400 MHz, DMSO-d₆): δ=5.27 (d, 2H), 5.87 (br. s, 2H), 7.32-7.35 (m, 1H), 7.51-7.58 (m, 2H), 7.77-7.85 (m, 1H), 7.45-7.50 (m, 1H).

### Example 30A

### Ethyl 6-chloro-8-[(3-fluoropyridin-2-yl)methoxy]-2-methylimidazo[1,2-a]pyridine-3-carboxylate

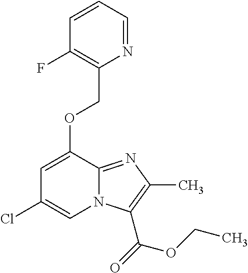 [SMILES: CCOC(=O)c1c(C)nc2c(OCc3ncccc3F)cc(Cl)cn12]

3.00 g (11.83 mmol) of 5-chloro-3-[(3-fluoropyridin-2-yl)methoxy]pyridine-2-amine from Example 29A and 9.73 g (59.13 mmol) of ethyl 2-chloro-3-oxobutanoate were dissolved in 72 ml of ethanol and, together with 4.5 g of 3 A molecular sieve, stirred under reflux for 6 days. The mixture was cooled and filtered and the filtrate was concentrated under reduced pressure. The residue obtained was purified by silica gel chromatography (mobile phase: cyclohexane/ethyl acetate gradient=4/1 to 2/1). This gave 2.0 g (46% of theory) of the target compound.

LC-MS (Method D): Rₜ=1.07 min

MS (ESIpos): m/z=364 (M+H)⁺

¹H-NMR (400 MHz, DMSO-d₆): δ=1.36 (t, 3H), 2.56 (s, 3H; overlapped with solvent peak), 4.37 (q, 2H), 5.48 (d, 2H), 7.36 (d, 1H), 7.57-7.63 (m, 1H), 7.83-7.90 (m, 1H), 8.50 (d, 1H), 8.92 (d, 1H).

### Example 31A

### 6-Chloro-8-[(3-fluoropyridin-2-yl)methoxy]-2-methylimidazo[1,2-a]pyridine-3-carboxylic acid

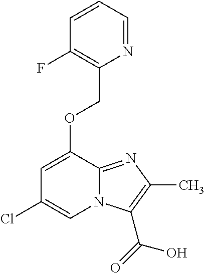 [SMILES: Cc1nc2c(OCc3ncccc3F)cc(Cl)cn2c1C(=O)O]

28.1 ml (28.1 mmol) of 1 M aqueous lithium hydroxide solution were added to 2.0 g (5.62 mmol) of ethyl 6-chloro-8-[(3-fluoropyridin-2-yl)methoxy]-2-methylimidazo[1,2-a]pyridine-3-carboxylate from Example 30A in 110 ml of THF/methanol (5:1), and the mixture was stirred at 40° C. for 2.5 h. Using 6 N aqueous hydrochloric acid, the reaction mixture, which had been cooled to RT, was adjusted to about pH 4, the solvent was concentrated to half its original volume and the precipitated solid was filtered off with suction and dried under reduced pressure. This gave 1.97 g (102% of theory) of the target compound (some of it possibly as hydrochloride salt).

LC-MS (Method D)): Rₜ=0.65 min

MS (ESIpos): m/z=336 (M+H)⁺

¹H-NMR (400 MHz, DMSO-d₆): δ=5.43-5.51 (m, 2H), 7.32 (d, 1H), 7.57-7.63 (m, 1H), 7.83-7.91 (m, 1H), 8.48-8.54 (min, 1H), 8.96-9.00 (min, 1H), 13.36 (br. s, 1H), [further signal under solvent peak].

### Example 32A

### rac-2-Amino-5,5,5-trifluoro-2-methylpentanonitrile

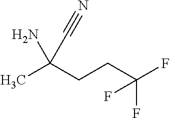

8.0 g (57.1 mmol) of 5,535-trifluoropentan-2-one [CAS Registry Number: 1341078-97-4; commercially available, or the methyl ketone can be prepared by literature methods which are known to those skilled in the art, for example via a) two stages from 4,4,4-trifluorobutanal according to Y. Bai et al. Angewandte Chemie 2012, 51, 4112-4116; K. Hiroi et al. Synlett 2001, 263-265; K. Mikami et al. 1982 Chemistry Letters, 1349-1352; b) or from 4,4,4-trifluorobutanoic acid according to A, A. Wube et al. Bioorganic and Medicinal Chemistry 2011, 19, 567-579; G. M. Rubottom et al. Journal of Organic Chemistry 1983, 48, 1550-1552; T. Chen et al. Journal of Organic Chemistry 1996, 61, 4716-4719. The product can be isolated by distillation or chromatography.] were initially charged in 47.8 ml of 2 N ammonia in methanol, 3.69 g (75.4 mmol) of sodium cyanide and 4.03 g (75.4 mmol) of ammonium chloride were added at room temperature and the mixture was stirred under reflux for 4 hours. The reaction mixture was cooled, diethyl ether was added and the solids present were filtered off. The solvent was distilled out of the filtrate under standard pressure. 8.7 g of the title compound (92% of theory) were obtained as residue, which was used in the subsequent stage without further purification.

GC-MS (Method H): Rₜ=1.90 min

MS (ESpos): m/z=151 (M−CH₃)⁺

### Example 33A

### rac-Benzyl (2-cyano-5,5,5-trifluoropentan-2-yl)carbamate

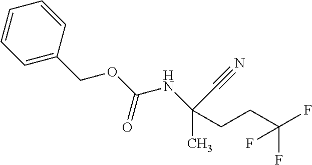 [SMILES: CC(C#N)(CCC(F)(F)F)NC(=O)OCc1ccccc1]

To an initial charge of 8.7 g (52.36 mmol) of rac-2-amino-5,5,5-trifluoro-2-methylpentanonitrile from Example 32A in 128 ml of tetrahydrofuran/water=9/1 were added 22.43 g (162.3 mmol) of potassium carbonate. At 0° C., 8.93 g (52.36 mmol) of benzyl chloroformate were slowly added dropwise. Then the mixture was allowed to warm up gradually to room temperature and stirred at room temperature overnight. The supernatant solvent was decanted off, the residue was twice stirred with 100 ml each time of tetrahydrofuran, and then the supernatant solvent was decanted off each time. The combined organic phases were concentrated and the crude product was purified by silica gel chromatography (mobile phase: cyclohexane/ethyl acetate gradient 9/1 to 4/1). 11.14 g of the title compound (68% of theory) were obtained.

LC-MS (Method D): Rₜ=1.01 min

MS (ESpos): m/z=301 (M+H)⁺

¹H-NMR (400 MHz, DMSO-d₆): δ [ppm]=1.58 (s, 3H), 2.08-2.21 (m, 2H), 2.24-2.52 (m, 2H), 5.09 (s, 2H), 7.29-7.41 (m, 5H), 8.17 (br. s, 1H).

### Example 34A

### ent-Benzyl (2-cyano-5,5,5-trifluoropentan-2-yl)carbamate (enantiomer A)

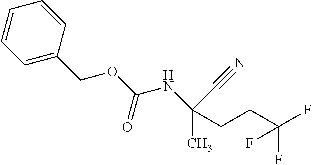 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 33A were separated into the enantiomers by preparative separation on a chiral phase [column: Daicel Chiralpak AZ-H, 5 μm, SFC, 250×50 mm, mobile phase: 94% carbon dioxide, 6% methanol, flow rate: 200 ml/min, temperature: 38° C., pressure: 135 bar; detection: 210 nm].

enantiomer A: 4.12 g (about 79% ee)

Rₜ=1.60 min. [SFC, Daicel Chiralpak AZ-H, 250×4.6 mm, 5 μm, mobile phase: 90% carbon dioxide, 10% methanol, flow rate: 3 ml/min, temperature: 30° C., detection: 220 nm].

LC-MS (Method D): Rₜ=1.01 min

MS (ESpos): m/z=301 (M+H)⁺

### Example 35A

### ent-Benzyl (2-cyano-5,5,5-trifluoropentan-2-yl)carbamate (enantiomer B)

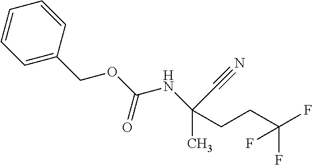 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 33A were separated into the enantiomers by preparative separation on a chiral phase [column: Daicel Chiralpak AZ-H, 5 μm, SFC, 250×50 mm, mobile phase: 94% carbon dioxide, 6% methanol, flow rate: 200 ml/min, temperature: 38° C., pressure: 135 bar; detection: 210 nm].

enantiomer B: 4.54 g (about 70% ee, purity about 89%)

Rₜ=1.91 min [SFC, Daicel Chiralpak AZ-H, 250×4.6 mm, 5 μm, mobile phase: 90% carbon dioxide, 10% methanol, flow rate: 3 ml/min, temperature: 30° C., detection: 220 nm].

LC-MS (Method D): Rₜ=1.01 min

MS (ESpos): m/z=301 (M+H)⁺

### Example 36A

### ent-Benzyl (1-amino-5,5,5-trifluoro-2-methylpentan-2-yl)carbamate (enantiomer A)

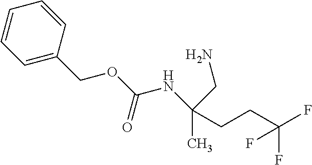 [SMILES: CC(CN)(CCC(F)(F)F)NC(=O)OCc1ccccc1]

4.12 g (13.17 mmol) of ent-benzyl (2-cyano-5,5,5-trifluoropentan-2-yl)carbonate (enantiomer A) from Example 34A were dissolved in 39 ml of 7 N ammonia solution in methanol, and 4 g of Raney nickel (50% aqueous slurry) were added under argon. The reaction mixture was hydrogenated in an autoclave at 20-30 bar overnight. Another I g of Raney nickel (50% aqueous slurry) was added and the reaction mixture was hydrogenated in an autoclave at 20-30 bar for 5 h. The reaction mixture was filtered through kieselguhr, rinsed with methanol and concentrated. 3.35 g (56% of theory; purity about 67%) of the target compound were obtained, which were used in the subsequent stage further without purification.

LC-MS (Method I): Rₜ=1.68 min

MS (ESpos): m/z=305 (M+H)⁺

¹H-NMR (400 MHz, DMSO-d₆): δ [ppm]=1.13 (s, 3H), 1.40 (br. s, 2H), 1.70-1.80 (m, 1H), 1.83-1.95 (m, 1H), 2.08-2.2 (m, 2H), 4.98 (s, 2H), 6.85 (br. s, 1H), 7.28-7.41 (m, 5H).

### Example 37A

### ent-Benzyl (1-amino-5,5,5-trifluoro-2-methylpentan-2-yl)carbamate (enantiomer B)

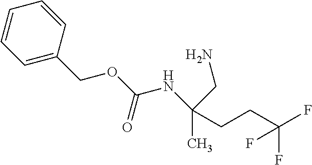 [SMILES: CC(CN)(CCC(F)(F)F)NC(=O)OCc1ccccc1]

4.54 g (13.45 mmol; purity about 89%) of ent-benzyl (2-cyano-5,5,5-trifluoropentan-2-yl)carbamate (enantiomer B) from Example 35A were dissolved in 39 ml of 7 N ammonia solution in methanol, and 5 g of Raney nickel (50% aqueous slurry) were added under argon. The reaction mixture was hydrogenated in an autoclave at 20-30 bar for 3 h. The reaction mixture was filtered through kieselguhr, rinsed with methanol and concentrated. 4.20 g (97% of theory; purity about 95%) of the target compound were obtained, which were used in the subsequent stage further without purification.

LC-MS (Method K): Rₜ=2.19 min

MS (ESpos): m/z=305 (M+H)⁺

¹H-NMR (400 MHz, DMSO-d₆): δ [ppm]=: 1.13 (s, 3H), 1.40 (br. s, 2H), 1.69-1.80 (m, 1H), 1.83-1.96 (m, 1H), 2.07-2.22 (m, 2H), 4.98 (s, 2H), 6.85 (br. s, 1H), 7.27-7.40 (m, 5H).

### Example 38A

### ent-Benzyl {5,5,5-trifluoro-1-[({8 [(3 fluoropyridin-2 yl)methoxy]-2,6-dimethyl imidazo[1,2-a]pyridin-3-yl}carbonyl)amino]-2-methylpentan-2-yl}carbamate trifluoroacetate (enantiomer B)

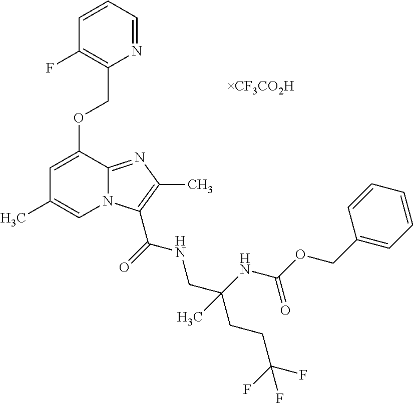 [SMILES: Cc1cc(OCc2ncccc2F)c2nc(C)c(C(=O)NCC(C)(CCC(F)(F)F)NC(=O)OCc3ccccc3)n2c1]

70 mg (0.20 mmol) of 8-[(3-fluoropyridin-2-yl)methoxy]-2,6-dimethylimidazo[1,2-a]pyridine-3-carboxylic acid hydrochloride from Example 26A, 93 mg (0.24 mmol) of HATU and 129 mg (1.00 mmol) of N,N-diisopropylethylamine were initially charged in 1.4 ml of DMF, and the mixture was stirred at RT for 20 min. Subsequently, 100 mg (0.31 mmol; purity about 95%) of ent-benzyl (1-amino-5,5,5-trifluoro-2-methylpentan-2-yl)carbamate (enantiomer B) from Example 37A were added and the mixture was stirred at RT overnight. The reaction solution was admixed with water and stirred at room temperature for 45 min. The solid present was filtered off, washed well with water and dried under high vacuum. The crude product was purified by preparative HPLC (RP-C18, mobile phase: acetonitrile/water gradient with addition of 0.1% TFA). This gave 98 mg (68% of theory) of the title compound.

LC-MS (Method D): Rₜ=0.93 min

MS (ESpos): m/z=602 (M-TFA+H)⁺

### Example 39A

### ent-Benzyl {1-[({6-chloro-8-[(3-fluoropyridin-2-yl)methoxy]-2-methylimidazo[1,2-a]pyridin-3-yl}carbonyl)amino]-5,5,5-trifluoro-2-methylpentan-2-yl}carbamate trifluoroacetate (enantiomer B)

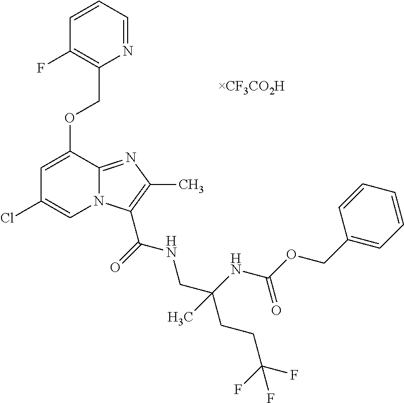 [SMILES: Cc1nc2c(OCc3ncccc3F)cc(Cl)cn2c1C(=O)NCC(C)(CCC(F)(F)F)NC(=O)OCc1ccccc1]

70 mg (0.21 mmol) of 6-chloro-8-[(3-fluoropyridin-2-yl)methoxy]-2-methylimidazo[1,2-a]pyridine-3-carboxylic acid from Example 31A, 87 mg (0.23 mmol) of HATU and 80 mg (0.63 mmol) of N,N-diisopropylethylamine were initially charged in 1.3 ml of DMF, and the mixture was stirred at RT for 20 min. Subsequently, 94 mg (0.29 mmol; purity about 95%) of ent-benzyl (1-amino-5,5,5-trifluoro-2-methylpentan-2-yl)carbamate (enantiomer B) from Example 37A were added and the mixture was stirred at RT overnight. Acetonitrile, water and TFA were added and the reaction solution was purified by preparative HPLC (RP-C18, mobile phase: acetonitrile/water gradient with addition of 0.1% TFA). This gave 103 mg (66% of theory) of the title compound.

LC-MS (Method D): Rₜ=1.13 min

MS (ESpos): m/z=622 (M-TFA+H)⁺

### Example 40A

### tert-Butyl {3-[({8-[(2,6-difluorobenzyl)oxy]-2,6-dimethylimidazo[1,2-a]pyridin-3-yl}carbonyl)amino]-2,2-difluorobenzyl}carbamate trifluoroacetate

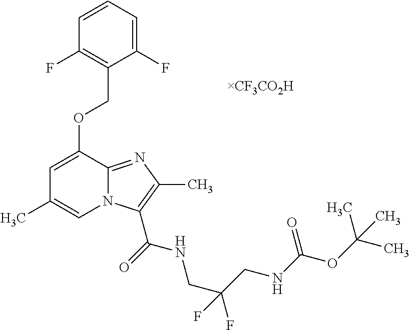 [SMILES: Cc1cc(OCc2c(F)cccc2F)c2nc(C)c(C(=O)NCC(F)(F)CNC(=O)OC(C)(C)C)n2c1]

100 mg (0.30 mmol) of 8-[(2,6-difluorobenzyl)oxy]-2,6-dimethylimidazo[1,2--a]pyridine-3-carboxylic acid from Example 24A, 149 mg (0.39 mmol) of HATU and 117 mg (0.90 mmol) of N,N-diisopropylethylamine were initially charged in 1.0 ml of DMF, and the mixture was stirred at RT for 20 min. 82 mg (0.39 mmol) of tert-butyl (3-amino-2,2-difluoropropyl)carbonate were then added, and the mixture was stirred at RT for 0.5 h. Acetonitrile, water and TFA were added and the reaction solution was purified by preparative HPLC (RP-C18, mobile phase: acetonitrile/water gradient with addition of 0.1% TFA). This gave 93 mg (79% of theory; purity 93%) of the title compound.

LC-MS (Method D): Rₜ=0.98 min

MS (ESpos): m/z=525 (M-TFA+H)⁺

### Example 41A

### rac-tert-Butyl {1-[({8-[(2,6-difluorobenzyl)oxy]-2-methyl-6-(1-methyl-1H-pyrazol-4-yl)imidazo[1,2-a]pyridin-3-yl}carbonyl)amino]-2-methylbutan-2-yl}carbamate

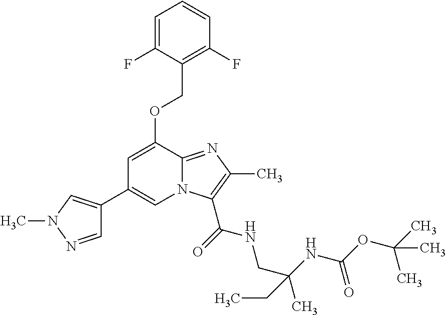 [SMILES: CCC(C)(CNC(=O)c1c(C)nc2c(OCc3c(F)cccc3F)cc(-c3cnn(C)c3)cn12)NC(=O)OC(C)(C)C]

A mixture of 100 mg (0.17 mmol) of rac-tert-butyl {1-[({6-bromo-8-[(2,6-difluorobenzyl)oxy]-2-methylimidazo[1,2-a]pyridin-3-yl}carbonyl)amino]-2-methylbutan-2-yl}carbamate (Example 5A), 43 mg (0.21 mmol) of 1-methyl-3-(4,4,5,5-tetramethyl-[1,3,2]dioxaborolan-2-yl)-1H-pyrazole, 14 mg (0.017 mmol) of 1,1′-bis(diphenylphosphino)ferrocenepalladium(II) dichloride/dichloromethane complex and 166 mg (0.51 mmol) of caesium carbonate in 0.5 ml of water and 2 ml of dioxane was degassed with argon for 5 min and stirred in a closed tube at 100° C. for 18 h. The reaction mixture was cooled to room temperature and the residue was partitioned between ethyl acetate and water. The organic phase was separated off, washed with saturated aqueous sodium chloride solution, dried over sodium sulphate, filtered and concentrated. The residue was purified by chromatography on silica gel (mobile phase: cyclohexane/ethyl acetate, gradient 0% to 50%). This gave 50 mg of the target product (50% of theory).

LC-MS (Method A): Rₜ=3.11 min; m/z=583 (M+H)⁺

¹H-NMR (400 MHz, CDCl₃): δ [ppm]=0.95 (t, 3H), 1.24 (s, 3H), 1.42 (s, 9H), 1.57-1.66 (m, 2H), 1.77-1.86 (m, 11H), 2.74 (s, 3H), 3.69-3.82 (m, 2H), 3.95 (s, 3H), 4.58 (s, 1H), 5.30 (s, 1H), 5.41 (s, 2H), 6.94 (dd, 3H), 7.20-7.24 (m, 1H), 7.34 (ddd, 1H), 7.67 (s, 1H), 7.77 (s, 1H), 9.21 (d, 1H).

### Example 42A

### rac-2-Amino-2-methyl-4-(trimethylsilyl)butanonitrile

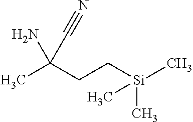

13.0 g (90.10 mmol) of 4-(trimethylsilyl)butan-2-one [commercially available or synthetically available according to R. Acerete et al. Journal of Organic Chemistry 2011, 76, 10129-10139] were initially charged in 25 ml of 7 N ammonia in methanol, 5.83 g (118.93 mmol) of sodium cyanide and 6.36 g (118.93 mmol) of ammonium chloride were added at room temperature and the mixture was stirred under reflux for 3 hours. The reaction mixture was cooled and the solid present was filtered off. The filtrate was used for the next step without further purification.

### Example 43A

### rac-Benzyl [2-cyano-4-(trimethylsilyl)butan-2-yl]carbamate

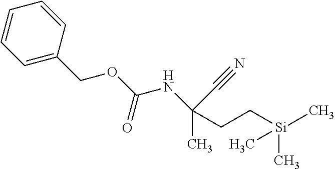 [SMILES: CC(C#N)(CC[Si](C)(C)C)NC(=O)OCc1ccccc1]

The crude solution of rac-2-amino-2-methyl-4-(trimethylsilyl)butanonitrile from Example 42A was initially charged in 16 ml of water, and 37.36 g (270.35 mmol) of potassium carbonate were added. At 0° C., 23.06 g (135.18 mmol) of benzyl chloroformate were slowly added dropwise. Then the mixture was allowed to warm up gradually to room temperature and stirred at room temperature overnight. The reaction mixture was filtered and the residue was washed repeatedly with tetrahydrofuran. The filtrate was concentrated and the crude product was purified by silica gel chromatography (mobile phase: cyclohexane/ethyl acetate=9/1). This gave 11.60 g of the title compound (42% of theory over two steps).

LC-MS (Method D): Rₜ=1.23 min

MS (ESpos): am/z=305 (M+H)⁺

¹H-NMR (400 MHz, DMSO-d₆): δ [ppm]=−0.01 (s, 9H), 0.45-0.67 (m, 2H), 1.52 (s, 3H), 1.73-1.90 (m, 2H), 2.24-2.52 (m, 2H), 5.08 (s, 2H), 7.29-7.44 (m, 51H), 7.94 (br. s, 1H).

### Example 44A

### ent-Benzyl [2-cyano-4-(trimethylsilyl)butan-2-yl]carbamate (enantiomer A)

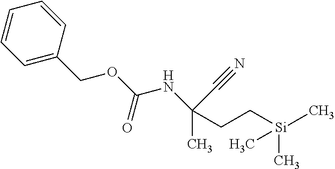 [SMILES: CC(C#N)(CC[Si](C)(C)C)NC(=O)OCc1ccccc1]

10.0 g of rac-benzyl [2-cyano-4-(trimethylsilyl)butan-2-yl]carbamate from Example 43A were separated into the enantiomers by preparative separation on a chiral phase [column: Daicel Chiralpak AY-H, 5 μm, 250×20 mm, mobile phase: 15% ethanol, 85% isohexane, flow rate: 20 ml/min, temperature: 30° C., detection: 220 nm].

enantiomer A: 4.19 g (>99% ee)

Rₜ=5.24 min [Daicel Chiralpak AY-H, 250×4.6 min, 5 μm, mobile phase: 10% ethanol, 90% isohexane, flow rate: 1 ml/min, temperature: 45° C., detection: 220 nm].

### Example 45A

### ent-Benzyl [2-cyano-4-(trimethylsilyl)butan-2-yl]carbamate (enantiomer B)

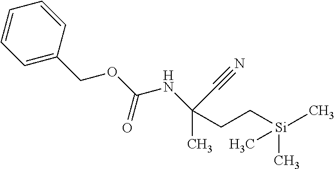 [SMILES: CC(C#N)(CC[Si](C)(C)C)NC(=O)OCc1ccccc1]

10.0 g of rac-benzyl [2-cyano-4-(trimethylsilyl)butan-2-yl]carbamate from Example 43A were separated into the enantiomers by preparative separation on a chiral phase [column: Daicel Chiralpak AY-H, 5 μm, 250×20 mm, mobile phase: 15% ethanol, 85% isohexane, flow rate: 20 ml/min, temperature: 30° C., detection: 220 nm].

enantiomer B: 4.24 g (>99% ee)

Rₜ=6.89 min [Daicel Chiralpak AY-H, 250×4.6 mm, 5 μm, mobile phase: 10% ethanol, 90% isohexane, flow rate: 1 ml/min, temperature: 45° C., detection: 220 nm].

### Example 46A

### ent-Benzyl [1-amino-2-methyl-4-(trimethylsilyl)butan-2-yl]carbamate (enantiomer A)

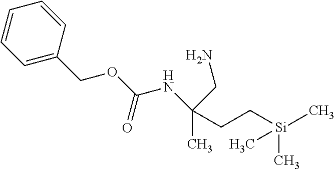 [SMILES: CC(CN)(CC[Si](C)(C)C)NC(=O)OCc1ccccc1]

2.0 g (6.57 mmol) of ent-benzyl [2-cyano-4-(trimethylsilyl)butan-2-yl]carbamate (enantiomer A) from Example 44A were dissolved in 31 ml of 7 N ammonia solution in methanol, and 2.44 g of Raney nickel (50% aqueous slurry) were added under argon. The reaction mixture was hydrogenated in an autoclave at 20-30 bar for 3 h. The reaction mixture was filtered through kieselguhr, rinsed with methanol and concentrated. This gave 1.80 g (87% of theory; purity 98%) of the target compound which was used without further purification for the next step.

LC-MS (Method M): Rₜ=1.66 min

MS (ESpos): m/z=309 (M+H)⁺

### Example 47A

### ent-Benzyl [1-amino-2-methyl-4-(trimethylsilyl)butan-2-yl]carbamate (enantiomer B)

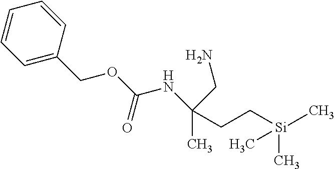 [SMILES: CC(CN)(CC[Si](C)(C)C)NC(=O)OCc1ccccc1]

2.0 g (6.57 mmol) of ent-benzyl [2-cyano-4-(trimethylsilyl)butan-2-yl]carbamate (enantiomer B) from Example 45A were dissolved in 31 ml of 7 N ammonia solution in methanol, and 2.44 g of Raney nickel (50% aqueous slurry) were added under argon. The reaction mixture was hydrogenated in an autoclave at 20-30 bar for 3 h. The reaction mixture was filtered through kieselguhr, rinsed with methanol and concentrated. This gave 1.72 g (83% of theory; purity 98%) of the target compound which was used without further purification for the next step.

LC-MS (Method D): Rₜ=0.78 min

MS (ESpos): m/z=309 (M+H)⁺

### Example 48A

### ent-Benzyl {1-[({8-[(2,6-difluorobenzyl)oxy]-2,6-dimethylimidazo[1,2-a]pyridin-3-yl}carbonyl)amino]-2-methyl-4-(trimethylsilyl)butan-2-yl}carbamate trifluoroacetate (enantiomer A)

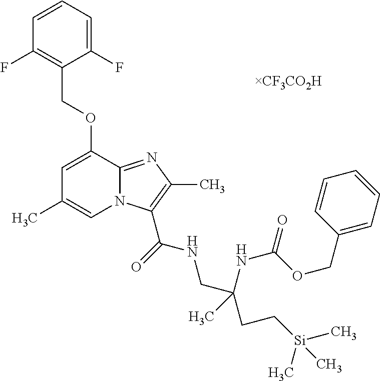 [SMILES: Cc1cc(OCc2c(F)cccc2F)c2nc(C)c(C(=O)NCC(C)(CC[Si](C)(C)C)NC(=O)OCc3ccccc3)n2c1]

An initial charge of 100 mg (0.30 mmol) of 8-[(2,6-difluorobenzyl)oxy]-2,6-dimethylimidazo[1,2-a]pyridine-3-carboxylic acid together with 149 mg (0.39 mmol) of HATU and 157 μl (0.90 mmol) of N,N-diisopropylethylamine in 0.99 ml of DMF was stirred at RT for 20 min, 123 mg (0.39 mmol, purity 98%) of ent-benzyl [1-amino-2-methyl-4-(trimethylsilyl)butan-2-yl]carbamate (enantiomer A) from Example 46A were then added, and the reaction solution was stirred at RT for 2 hours. 19 mg (0.06 mmol) of ent-benzyl [1-amino-2-methyl-4-(trimethylsilyl)butan-2-yl]carbamate (enantiomer A) from Example 46A were then added, and the mixture was stirred at RT for a further 2 hours. The reaction solution was taken up in acetonitrile, water and TFA and purified by preparative HPLC (RP18 column, mobile phase: acetonitrile/water gradient with addition of 0.1% TFA). The product fractions were combined, concentrated and dried under high vacuum. This gave 151 mg of the target compound (66% of theory, purity 97%).

LC-MS (Method D): Rₜ=1.30 min

MS (ESpos): m/z=623 (M-TFA+H)⁺

### Example 49A

### ent-Benzyl {1-[({8-[(2,6-difluorobenzyl)oxy]-2,6-dimethylimidazo[1,2-a]pyridin-3-yl}carbonyl)amino]-2-methyl-4-(trimethylsilyl)butan-2-yl}carbamate trifluoroacetate (enantiomer B)

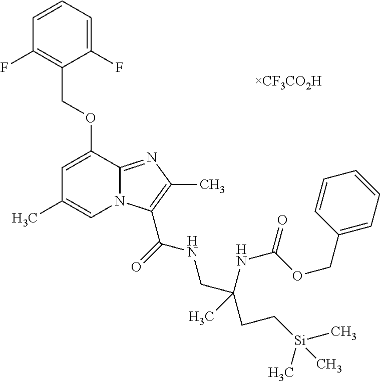 [SMILES: Cc1cc(OCc2c(F)cccc2F)c2nc(C)c(C(=O)NCC(C)(CC[Si](C)(C)C)NC(=O)OCc3ccccc3)n2c1]

An initial charge of 100 mg (0.30 mmol) of 8-[(2,6-difluorobenzyl)oxy]-2,6-dimethylimidazo[1,2-a]pyridine-3-carboxylic acid together with 149 mg (0.39 mmol) of HATU and 157 μl (0.90 mmol) of N,N-diisopropylethylamine in 0.99 ml of DMF was stirred at room temperature for 20 min. 123 mg (0.39 mmol, purity 98%) of ent-benzyl [1-amino-2-methyl-4-(trimethylsilyl)butan-2-yl]carbamate (enantiomer B) from Example 47A were then added, and the reaction solution was stirred at RT for 2 hours. 19 mg (0.06 mmol) of ent-benzyl [1-amino-2-methyl-4-(trimethylsilyl)butan-2-yl]carbamate (enantiomer B) from Example 47A were then added, and the mixture was stirred at RT for a further 2 hours. The reaction solution was taken up in acetonitrile, water and TFA and purified by preparative HPLC (RP18 column, mobile phase: acetonitrile/water gradient with addition of 0.1% TFA). The product fractions were combined, concentrated and dried under high vacuum. This gave 168 mg of the target compound (75% of theory, purity 99%).

LC-MS (Method D): Rₜ=1.28 min

MS (ESpos): m/z=623 (M-TFA+H)⁺

### Example 50A

### rac-2-Amino-2-methyl-5-(trimethylsilyl)pentanonitrile

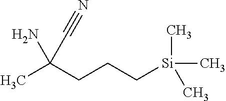

24.8 g (156.6 mmol) of 5-(trimethylsilyl)pentan-2-one were initially charged in 45 ml of 7 N ammonia in methanol, 10.1 g (206.8 mmol) of sodium cyanide and 10.9 g (203.6 mmol) of ammonium chloride were added at room temperature and the mixture was stirred under reflux for 3 hours. The reaction solution was cooled, 100 ml of THF were added and the solid was filtered off and washed twice with THF. The filtrate was used for the next step without further purification.

### Example 51A

### rac-Benzyl [2-cyano-5-(trimethylsilyl)pentan-2-yl]carbamate

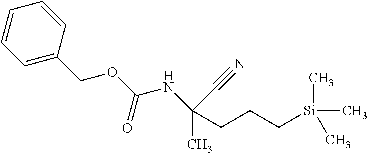 [SMILES: CC(C#N)(CCC[Si](C)(C)C)NC(=O)OCc1ccccc1]

The crude solution of rac-2-amino-2-methyl-5-(trimethylsilyl)pentanonitrile from Example 50A was initially charged in 50 ml of water, and 64.95 g (469.95 mmol) of potassium carbonate were added. At 0° C., 33.55 ml (234.98 mmol) of benzyl chloroformate were slowly added dropwise. Then the mixture was allowed to warm up gradually to room temperature and stirred at room temperature overnight. The reaction mixture was filtered and the residue was washed repeatedly with THF The filtrate was concentrated and the residue was purified by silica gel chromatography (mobile phase: cyclohexane/ethyl acetate=20/1). This gave 29.0 g of the title compound (58% of theory).

LC-MS (Method H): Rₜ=7.16 min

MS (ESpos): m/z=183 (M-C₆H₅CH₂CO₂[Cbz])

### Example 52A

### ent-Benzyl [2-cyano-5-(trimethylsilyl)pentan-2-yl]carbamate (enantiomer A)

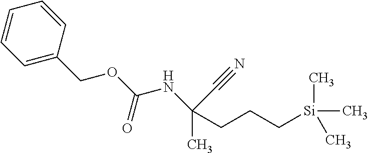 [SMILES: CC(C#N)(CCC[Si](C)(C)C)NC(=O)OCc1ccccc1]

8.49 g of rac-benzyl [2-cyano-5-(trimethylsilyl)pentan-2-yl]carbamate from Example 51A were separated into the enantiomers by preparative separation on a chiral phase [column: Chiralcel OD-H 5 μm 250×50 mm; mobile phase: 94% carbon dioxide, 6% isopropanol, flow rate: 175 ml/min, temperature: 38° C., detection: 210 nm].

enantiomer A: 3.53 g (>99% ee)

Rₜ=1.21 min [Chiralcel OD-3, 100×4.6 mm, 5 μm, mobile phase: 90% carbon dioxide, 10% isopropanol, flow rate: 3 ml/min, temperature: 40° C., detection: 210 nm].

### Example 53A

### ent-Benzyl [2-cyano-5-(trimethylsilyl)pentan-2-yl]carbamate (enantiomer B)

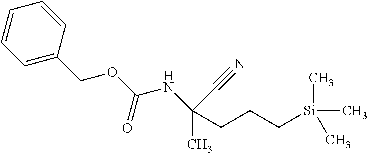 [SMILES: CC(C#N)(CCC[Si](C)(C)C)NC(=O)OCc1ccccc1]

8.49 g of rac-benzyl [2-cyano-5-(trimethylsilyl)pentan-2-yl]carbamate from Example 51A were separated into the enantiomers by preparative separation on a chiral phase [column: Chiralcel OD-H 5 μm 250×50 mm: mobile phase: 94% carbon dioxide, 6% isopropanol, flow rate: 175 ml/min, temperature: 38° C., detection: 210 nm].

enantiomer B: 3.53 g (>98% ee)

Rₜ=1.35 min [Chiralcel OD-3, 100×4.6 mm, 5 μm, mobile phase: 90% carbon dioxide, 10% isopropanol, flow rate: 3 ml/min, temperature: 40° C., detection: 210 nm].

### Example 54A

### ent-Benzyl [1-amino-2-methyl-5-(trimethylsilyl)pentan-2-yl]carbamate (enantiomer A)

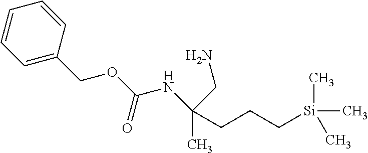 [SMILES: CC(CN)(CCC[Si](C)(C)C)NC(=O)OCc1ccccc1]

2.50 g (7.61 mmol, purity 97%) of ent-benzyl [2-cyano-5-(trimethylsilyl)pentan-2-yl]carbamate (enantiomer A) from Example 52A were dissolved in 36 ml of 7 N ammonia solution in methanol, and 2.83 g of Raney nickel (50% aqueous slurry) were added under argon. The reaction mixture was hydrogenated in an autoclave at 25 bar for 3 h. The reaction mixture was filtered through kieselguhr, washed with methanol and concentrated. This gave 1.14 g (45% of theory, purity 98%) of the target compound.

LC-MS (Method O): Rₜ=1.42 min

MS (ESpos): m/z=323 (M+H)⁺

### Example 55A

### ent-Benzyl [1-amino-2-methyl-5-(trimethylsilyl)pentan-2-yl]carbamate (enantiomer B)

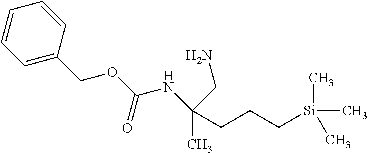 [SMILES: CC(CN)(CCC[Si](C)(C)C)NC(=O)OCc1ccccc1]

2.50 g (7.61 mmol, purity 97%) of ent-benzyl [2-cyano-5-(trimethylsilyl)pentan-2-yl]carbamate (enantiomer B) from Example 53A were dissolved in 36 ml of 7 N ammonia solution in methanol, and 2.83 g of Raney nickel (50% aqueous slurry) were added under argon. The reaction mixture was hydrogenated in an autoclave at 20-30 bar for 3 h. The reaction mixture was filtered through kieselguhr, washed with methanol and concentrated. This gave 2.34 g (84% of theory; purity 88%) of the target compound.

LC-MS (Method O): Rₜ=1.41 min

MS (ESpos): m/z=323 (M+H)³⁰

### Example 56A

### ent-Benzyl {1-[({8-[(2,6-difluorobenzyl)oxy]-2,6-dimethylimidazo[1,2-a]pyridin-3-yl}carbonyl)-amino]-2-methyl-5-(trimethylsilyl)pentan-2-yl}carbamate trifluoroacetate (enantiomer A)

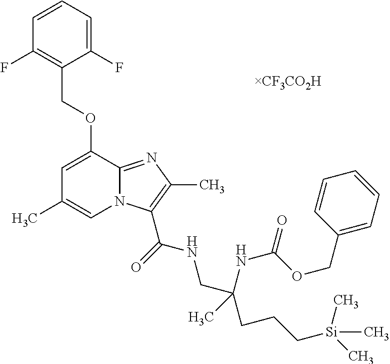 [SMILES: Cc1cc(OCc2c(F)cccc2F)c2nc(C)c(C(=O)NCC(C)(CCC[Si](C)(C)C)NC(=O)OCc3ccccc3)n2c1]

100 mg (0.30 mmol) of 8-[(2,6-difluorobenzyl)oxy]-2,6-dimethylimidazo[1,2-a]pyridine-3-carboxylic acid from Example 24A were initially charged together with 120 mg (0.32 mmol) of HATU and 262 μl (1.51 mmol) of N,N-diisopropylethylamine in 1.0 ml of DMF, and the mixture was stirred at room temperature for 10 min. 107 mg (0.33 mmol) of ent-benzyl [1-amino-2-methyl-5-(trimethylsilyl)pentan-2-yl]carbamate (enantiomer A) from Example 54A were then added, and the reaction solution was stirred at RT overnight. The mixture was then diluted with acetonitrile and water, TFA was added and the mixture was purified by preparative HPLC (RP18 column, mobile phase: acetonitrile/water gradient with addition of 0.1% TFA). The product fractions were combined and concentrated. This gave 149 mg of the target compound (65% of theory).

LC-MS (Method D): Rₜ=129 min

MS (ESpos): m/z=637 (M-TFA+H)⁺

### Example 57A

### ent-Benzyl {1-[({8-[(2,6-difluorobenzyl)oxy]-2,6-dimethylimidazo[1,2-a]pyridin-3-yl}carbonyl)-amino]-2-methyl-5-(trimethylsilyl)pentan-2-yl}carbamate trifluoroacetate (enantiomer B)

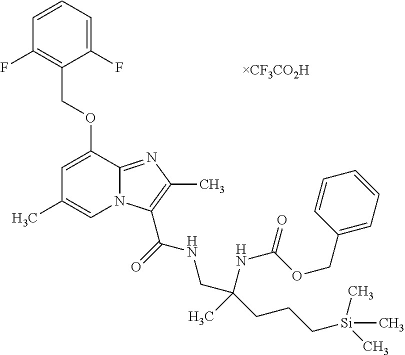 [SMILES: Cc1cc(OCc2c(F)cccc2F)c2nc(C)c(C(=O)NCC(C)(CCC[Si](C)(C)C)NC(=O)OCc3ccccc3)n2c1]

100 mg (0.30 mmol) of 8-[(2,6-difluorobenzyl)oxy]-2,6-dimethylimidazo[1,2-a]pyridine-3-carboxylic acid from Example 24A were initially charged together with 120 mg (0.32 mmol) of HATU and 262 μl (1.51 mmol) of N,N-diisopropylethylamine in 1.0 ml of DMF, and the mixture was stirred al room temperature for 10 in. 121 mg (0.33 mmol, purity 88%) of ent-benzyl [1-amino-2-methyl-5-(trimethylsilyl)pentan-2-yl]carbamate (enantiomer B) from Example 55A were then added, and the reaction solution was stirred at RT overnight. The mixture was then diluted with acetonitrile and water, TFA was added and the mixture was purified by preparative HPLC (RP18 column, mobile phase: acetonitrile/water gradient with addition of 0.1% TFA). The product fractions were combined and concentrated. This gave 189 mg of the target compound (83% of theory).

LC-MS (Method O): Rₜ=2.58 min

MS (ESpos): m/z=637 (M-TFA+H)⁺

### Example 58A

### 3-Oxocyclobutanecarbonitrile

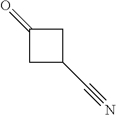

25.0 g (268.4 mmol) of 3-methylenecyclobutanecarbonitriie and 1.23 g (5.91 mmol) of ruthenium(III) chloride were initially charged in 500 ml of dichloromethane, 500 ml of acetonitrile and 800 ml of water. 235.4 g (1100.6 mmol) of sodium periodate were then added a little at a time with ice cooling, and the mixture was stirred at RT overnight. The reaction mixture was filtered and the aqueous phase was extracted with dichloromethane. The combined organic phases were eluted through a short silica gel frit washing with a little dichloromethane/methanol (20/1), and the filtrate was concentrated. The residue was diluted with 200 ml of dichloromethane and washed first with saturated aqueous sodium sulphate solution and then with saturated aqueous sodium chloride solution. The organic phase was dried over sodium sulphate, filtered and concentrated. The oily residue was stirred with 100 ml of cold diethyl ether, and the precipitated solid was filtered off and washed with a little cold diethyl ether. This gave 13.61 g (53% of theory) of the title compound. The mother liquor was concentrated and cooled and the precipitated solid was filtered off with suction and washed with a little diethyl ether. This gave another 0.96 g (4% of theory) of the title compound.

GC-MS (Method H): Rₜ=2.76 min

MS (ESpos): m/z=95 (M)⁺

### Example 59A

### 3,3-Difluorocyclobutanecarbonitrile

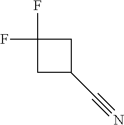

Under argon, 14.57 g (153.2 mmol) of 3-oxocyclobutanecarbonitrile from Example 58A were initially charged in 200 ml of absolute dichloromethane, 40.48 ml (306.4 mmol) of diethylaminosulphur trifluoride dissolved in 50 ml of dichloromethane were added at 0° C. and the mixture was stirred at RT overnight. A little at a time, the reaction mixture was then poured into a saturated aqueous sodium bicarbonate solution, cooled to 0° C., and stirred for 30 minutes. The organic phase was separated off and the aqueous phase was extracted with 200 ml of dichloromethane. The combined organic phases were twice washed with water, dried over sodium sulphate, filtered and concentrated. This gave 15.2 g (85% of theory) of the title compound.

GC-MS (Method H): Rₜ=1.43 min

MS (ESpos): m/z=98 (M−F)⁺

### Example 60A

### Ethyl 3-(3,3-difluorocyclobutyl)-3-oxopropanoate

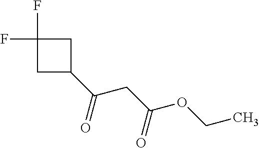

Under argon, 0.208 ml (3.20 mmol) of methanesulphonic acid were initially charged in 80 ml absolute THF, 20.93 g (320.2 mmol) of zinc were added and the mixture was stirred under reflux for 10 min. 15.0 g (128.1 mmol) of 3,3-difluorocyclobutanecarbonitrile from Example 59A were added and the mixture was stirred under reflux for a further 10 min. The heating bath was switched off and a solution of 28.41 ml (256.2 mmol) of ethyl bromoacetate in 30 ml of absolute THF was added dropwise over 2.5 hours. The mixture was stirred at reflux for 15 min and then allowed to stand at RT overnight. With ice cooling, 100 ml of a 10% strength aqueous hydrochloric acid were added dropwise, and the mixture was stirred overnight and allowed to warm to room temperature. 500 ml of water were added and the mixture was extracted three times with in each case 150 ml of ethyl acetate. The combined organic phases were dried over sodium sulphate, filtered and concentrated. After drying under high vacuum, the residue was purified by silica gel chromatography (cyclohexane/ethyl acetate=9/1). This gave 4.37 g (12% of theory, purity about 76%) of the title compound.

GC-MS (Method H): Rₜ=3.43 min

MS (ESpos): m/z=206 (M)⁺

### Example 61A

### Ethyl 2-chloro-3-(3,3-difluorocyclobutyl)-3-oxopropanoate

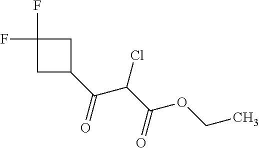

Under argon, 4.37 g (16.1 mmol, purity about 76%) of ethyl 3-(3,3-difluorocyclobutyl)-3-oxopropanoate from Example 60A were dissolved in 30.2 ml of absolute dichloromethane, and 1.94 ml (24.2 mmol) of sulphuryl dichloride were added. The mixture was then stirred at RT overnight. The reaction solution was diluted with 100 ml of ethyl acetate and washed first with 50 ml of water and then with 50 ml of saturated aqueous sodium chloride solution. The organic phase was dried over sodium sulphate and filtered and the filtrate was concentrated. After brief drying under high vacuum, the residue was purified by silica gel chromatography (cyclohexane/ethyl acetate=10/1). This gave 3.25 g (68% of theory, purity 81%) of the title compound.

GC-MS (Method H): Rₜ=3.75 min

MS (ESpos): m/z=240 (M)⁺

### Example 62A

### Ethyl 8-[(2,6-difluorobenzyl)oxy]-2-(3,3-difluorocyclobutyl)-6-methylimidazo[1,2-a]pyridine-3-carboxylate

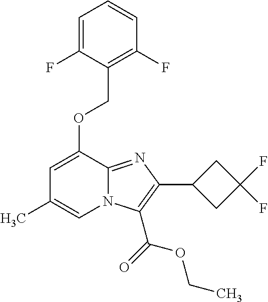 [SMILES: CCOC(=O)c1c(C2CC(F)(F)C2)nc2c(OCc3c(F)cccc3F)cc(C)cn12]

400 mg (1.60 mmol) of 3-[(2,6-difluorobenzyl)oxy]-5-methylpyridine-2-amine were dissolved in 15 ml of ethanol, and 950 mg (3.12 mmol, purity 81%) of ethyl 2-chloro-3-(3,3-difluorocyclobutyl)-3-oxopropanoate from Example 61A and 636 mg of 3 A molecular sieve were added. The reaction mixture was then stirred in the microwave at 150° C. for 4 hours. 475 mg (1.60 mmol, purity 81%) of ethyl 2-chloro-3-(3,3-difluorocyclobutyl)-3-oxopropanoate from Example 61A were added and the mixture was stirred in the microwave at 150° C. for another 2 hours. The reaction suspension was diluted with 50 ml of cyclohexane and filtered. The filtrate was concentrated, dried under high vacuum and purified by silica gel chromatography (mobile phase: cyclohexane/ethyl acetate gradient: 50/1 to 5/1). This gave 127 mg of the target compound (18% of theory, purity 99%). The filtration residue was extracted with ethyl acetate twice and filtered off and the filtrate was concentrated and dried under high vacuum. This gave another 206 mg of the target compound (29% of theory, purity 99%).

LC-MS (Method N): Rₜ=1.67 min

MS (ESpos): m/z=437 (M+H)⁺

### Example 63A

### 8-[(2,6-Difluorobenzyl)oxy]-2-(3,3-difluorocyclobutyl)-6-methylimidazo[1,2-a]pyridine-3-carboxylic acid

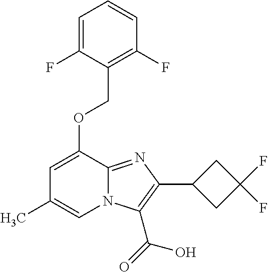 [SMILES: Cc1cc(OCc2c(F)cccc2F)c2nc(C3CC(F)(F)C3)c(C(=O)O)n2c1]

330 mg (0.75 mmol) of ethyl 8-[(2,6-difluorobenzyl)oxy]-2-(3,3-difluorocyclobutyl)-6-methylmidazo[1,2-a]pyridine-3-carboxylate from Example 62A were dissolved in 4 ml of THF, 54 mg (2.25 mmol) of lithium hydroxide in 2.2 ml of water were added and the mixture was stirred at RT overnight. Another 54 mg (2.25 mmol) of lithium hydroxide in 2.2 ml of water were added, and the mixture was stirred at 50° C. for one hour. 1.87 ml of 2N aqueous sodium hydroxide solution and 4 ml of dioxane/methanol (1:1) were added and the mixture was stirred at 50° C. for 2.5 hours. The reaction mixture was concentrated slightly and acidified with 1 N aqueous hydrochloric acid. The solid formed was filtered off, washed with a little water and diethyl ether and dried under high vacuum. This gave 219 mg (69% of theory, purity 97%) of the title compound.

LC-MS (Method N): Rₜ=1.31 min

MS (ESpos): m/z=409 (M+H)⁺

### Example 64A

### ent-Benzyl {1-[({8-[(2,6-difluorobenzyl)oxy]-2-(3,3-difluorocyclobutyl)-6-methylimidazo[1,2-a]pyridin-3-yl}carbonyl)amino]-5,5,5-trifluoro-2-methylpentan-2-yl}carbamate trifluoroacetate (enantiomer B)

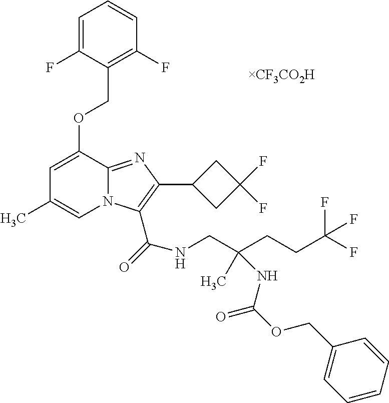 [SMILES: Cc1cc(OCc2c(F)cccc2F)c2nc(C3CC(F)(F)C3)c(C(=O)NCC(C)(CCC(F)(F)F)NC(=O)OCc3ccccc3)n2c1]

30 mg (0.071 mmol, purity 97%) of 8-[(2,6-difluorobenzyl)oxy]-2-(3,3-difluorocyclobutyl)-6-methylimidazo[1,2-a]pyridine-3-carboxylic acid from Example 63A were initially charged together with 30 mg (0.078 mmol) of HATU and 62 μl (0.36 mmol) of N,N-diisopropylethylamine in 0.3 ml of DMF, and the mixture was stirred at room temperature for 20 min. 30 mg (0.086 mmol; purity 88%) of ent-benzyl (1-amino-5,5,5-trifluoro-2-methylpentan-2-yl)carbamate (enantiomer B) from Example 37A were added to the reaction solution and the mixture was stirred at RT for 2 h. Acetonitrile, water and TFA were added and the reaction solution was purified by preparative HPLC (RP18 column, mobile phase: methanol/water gradient with addition of 0.1% TFA). The product fractions were combined and concentrated. This gave 55 mg of the target compound (89% of theory, purity 93%).

LC-MS (Method N): Rₜ=1.64 min

MS (ESpos): m/z=695 (M-TFA+H)⁺

### Example 65A

### Ethyl 8-[(2,6-difluorobenzyl)oxy]-2-isopropyl-6-methylimidazo[1,2-a]pyridine-3-carboxylate

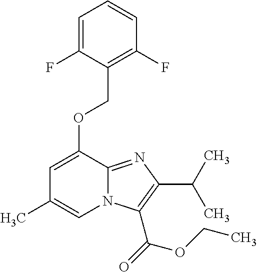 [SMILES: CCOC(=O)c1c(C(C)C)nc2c(OCc3c(F)cccc3F)cc(C)cn12]

Under argon, 1.50 g (5.99 mmol) of 3-[(2,6-difluorobenzyl)oxy]-5-methylpyridine-2-amine were initially charged in 30 ml of ethanol, 9.24 g (47.95 mmol) of ethyl 2-chloro-4-methyl-3-oxopentanoate [known from the literature, e.g. in WO2006/91506, Example N, step 1] and 0.30 g of molecular sieve 3 A were added and the mixture was stirred under reflux for 5 days. The reaction solution was concentrated, and 100 ml of water and 100 ml of ethyl acetate were added. The aqueous phase was re-extracted with ethyl acetate, and the combined organic phases were dried and concentrated. The residue was purified by silica gel chromatography (mobile phase: cyclohexane/ethyl acetate gradient=95/5 to 9/1 to 8/2). This gave 0.60 g (26% of theory) of the title compound.

LC-MS (Method L): Rₜ=2.64 min

MS (ESpos): m/z=389 (M+H)⁺

### Example 66A

### 8-[(2,6-Difluorobenzyl)oxy]-2-isopropyl-6-methylimidazo [1,2-a]pyridine-3-carboxylic acid

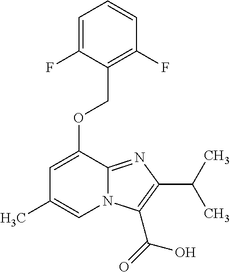 [SMILES: Cc1cc(OCc2c(F)cccc2F)c2nc(C(C)C)c(C(=O)O)n2c1]

0.60 g (1.54 mmol) of ethyl 8-[(2,6-difluorobenzyl)oxy]-2-isopropyl-6-methylimidazo[1,2-a]pyridine-3-carboxylate from Example 65A were dissolved in 28.2 ml of THF, 5.64 ml of methanol and 7.56 ml of water, 0.324 g (7.72 mmol) of lithium hydroxide monohydrate were added and the mixture was stirred at RT for 16 hours. The reaction mixture was then stirred at 40° C. for 4 hours. The organic solvent was removed on a rotary evaporator and the aqueous solution was adjusted to pH 2 using semiconcentrated aqueous hydrochloric acid. The mixture was then extracted with dichloromethane/methanol, the combined organic phases were dried and filtered and the filtrate was concentrated. This gave 170 mg (31% of theory) of the title compound.

LC-MS (Method D): R₁=0.86 min

MS (ESpos): m/z=361 (M+H)⁺

### Example 67A

### ent-Benzyl {1-[({8-[(2,6-difluorobenzyl)oxy]-2-isopropyl-6-methylimidazo[1,2-a]pyridin-3-yl}carbonyl)amino]-5,5,5-trifluoro-2-methylpentan-2-yl}carbamate trifluoroacetate (enantiomer B)

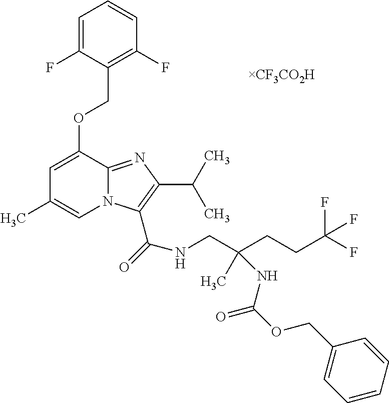 [SMILES: Cc1cc(OCc2c(F)cccc2F)c2nc(C(C)C)c(C(=O)NCC(C)(CCC(F)(F)F)NC(=O)OCc3ccccc3)n2c1]

50 mg (0.139 mmol) of 8-[(2,6-difluorobenzyl)oxy]-2-isopropyl-6-methylimidazo[1,2-a]pyridine-3-carboxylic acid from Example 66A were initially charged together with 58 mg (0.153 mmol) of HATU and 73 μl (0.416 mmol) of N,N-diisopropylethylamine in 0.41 ml of DMF, and the mixture was stirred at room temperature for 10 min. 51 mg (0.166 mmol) of ent-benzyl (1-amino-5,5,5-trifluoro-2-methylpentan-2-yl)carbamate (enantiomer B) from Example 37A were added to the reaction solution and the mixture was stirred at RT overnight. TFA and methanol were added and the reaction solution was purified by preparative HPLC (RP18 column, mobile phase: acetonitrile/water gradient with addition of 0.1% TFA). The product fractions were combined, concentrated and lyophilized. This gave 37 mg of the target compound (35% of theory).

LC-MS (Method N): Rₜ=1.47 min

MS (ESpos): m/z=647 (M-TFA+H)

### Example 68A

### Ethyl 8-((2,6-difluorobenzyl)oxy)-2-hydroxy-6-methylimidazo[1,2-a]pyridine-3-carboxylate

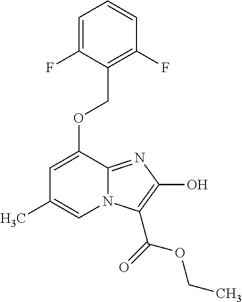 [SMILES: CCOC(=O)c1c(O)nc2c(OCc3c(F)cccc3F)cc(C)cn12]

2.7 g (32.0 mmol) of sodium bicarbonate were added to a mixture of 4.0 g (16.0 mmol) of 3-[(2,6-difluorobenzyl)oxy]-5-methylpyridine-2-amine [described in WO2014/068099, Example No. 323A] and 13.6 ml (9.0 mmol) diethyl 2-bromopropanedioate in 30 ml of acetonitrile, and the reaction mixture was stirred at 80° C. for 4 hours. After cooling to room temperature, the solvent was removed, and water and diethyl ether were added. The mixture was filtered off and the solid obtained was washed with water and diethyl ether and dried under reduced pressure. This gave 4.33 g of the target compound (75% of theory).

¹H-NMR (300 MHz, DMSO-d₆): δ [ppm]=8.79 (s, 1H), 7.62-7.52 (m, 1H), 7.24-7.18 (m, 3H), 5.30 (s, 21H), 4.23 (q, 2H), 2.35 (s, 3H), 1.27 (t, 3H).

### Example 69A

### Ethyl 2-chloro-8-((2,6-difluorobenzyl)oxy)-6-methylimidazo[1,2-a]pyridine-3-carboxylate

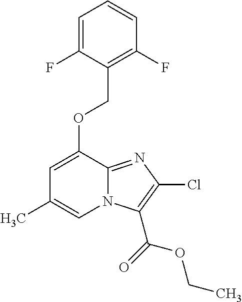 [SMILES: CCOC(=O)c1c(Cl)nc2c(OCc3c(F)cccc3F)cc(C)cn12]

7.7 ml (82.8 mmol) of phosphorus oxychloride were added to 3 g (8.3 mmol) of ethyl 8-[(2,6-difluorophenyl)methoxy]-2-hydroxy-6-methylimidazo[1,2-a]pyridine-3-carboxylate from Example 68A, and the reaction mixture was heated in a pressure tube at 110° C. for 6 days. After cooling to room temperature, the phosphorus oxychloride was evaporated under reduced pressure and water was added slowly to the residue, with the reaction mixture being cooled in an ice bath. The organic components were extracted with ethyl acetate, and the combined organic phases were dried over sodium sulphate, filtered and concentrated. The residue was purified by chromatography on silica gel (mobile phase: dichloromethane). This gave 840 mg of the target product (24% of theory).

¹H-NMR (300 MHz, DMSO-d₆): δ [ppm]=8.71 (s, 1H), 7.61-7.55 (m, 1H), 7.25-7.19 (m, 3H), 5.31 (s, 2H), 4.36 (q, 2H), 2.38 (s, 3H), 1.34 (t, 3H).

### Example 70A

### Ethyl 8-((2,6-difluorobenzyl)oxy)-2-methoxy-6-methylimidazo [1,2-a]pyridine-3-carboxylate

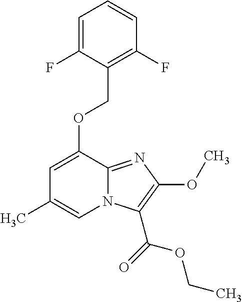 [SMILES: CCOC(=O)c1c(OC)nc2c(OCc3c(F)cccc3F)cc(C)cn12]

3.62 g (13.1 mmol) of silver carbonate and 0.90 ml (14.4 mmol) of methyl iodide were added to a suspension of 4.76 g (13.1 mmol) of ethyl 8-[(2,6-difluorophenyl)methoxy]-2-hydroxy-6-methylimidazo[1,2-a]pyridine-3-carboxylate from Example 68A in 95 ml of DMF, and the reaction mixture was stirred at room temperature in the dark for 16 hours. Water was added and the organic components were extracted with dichloromethane. The combined organic phases were washed with water, dried over sodium sulphate, filtered and concentrated. The residue was purified by chromatography on silica gel (mobile phase: dichloromethane). This gave 2.6 g of the target product (52% of theory).

¹H-NMR (300 MHz, DMSO-d₆): δ [ppm]=8.73 (s, 1H), 7.60-7.55 (m, 1H), 7.26-7.15 (m, 3H), 5.29 (s, 2H), 4.27 (q, 2H), 3.95 (s, 3H), 2.35 (s, 3H), 1.27 (t, 3H).

### Example 71A

### 2-Chloro-8-((2,6-difluorobenzyl)oxy)-6-methylimidazo[1,2-a]pyridine-3-carboxylic acid

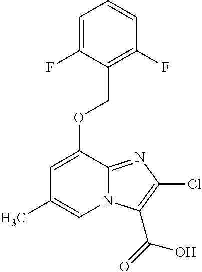 [SMILES: Cc1cc(OCc2c(F)cccc2F)c2nc(Cl)c(C(=O)O)n2c1]

8.6 ml (8.6 mmol) of a 1 M solution of sodium hydroxide in water were added dropwise to a suspension of 820 mg (2.2 mmol) of ethyl 2-chloro-8-[(2,6-difluorophenyl)methoxy]-6-methylimidazo[1,2-a]pyridine-3-carboxylate from Example 69A in a mixture of 8 ml of methanol and 8 ml of THF, and the reaction mixture was stirred at room temperature for 24 hours. The solvents were evaporated, and water was then added. The aqueous phase was acidified to pH 2 by addition of a 1 M solution of hydrochloric acid, and the product was extracted with ethyl acetate. The combined organic phases were dried over sodium sulphate, filtered and concentrated. This gave 480 mg of the target compound (63% of theory).

LC-MS (Method P): Rₜ=1.31 min; m/z=353 (M+H)⁺

¹H-NMR (300 MHz, DMSO-d₆): δ [ppm]=8.76 (s, 1H), 7.63-7.53 (m, 1H), 7.26-7.20 (m, 3H), 5.30 (s, 2H), 2.37 (s, 3H).

### Example 72A

### 8-((2,6-Difluorobenzyl)oxy)-2-methoxy-6-methylimidazo [1,2-a]pyridine-3-carboxylic acid

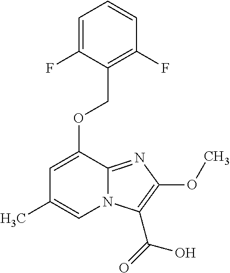

10.6 ml (10.63 mmol) of a 1 M solution of sodium hydroxide in water were added to a suspension of 2.0 g (5.31 mmol) of ethyl 8-[(2,6-difluorophenyl)methoxy]-2-methoxy-6-methylimidazo[1,2-a]pyridine-3-carboxylate from Example 70A in 25 ml of DMSO, and the reaction mixture was stirred at 60° C. for 10 hours. After cooling to room temperature, a 1 M solution of hydrochloric acid was added slowly to adjust the pH of the mixture to 1-2. The solid formed was filtered off, washed with water and dried. This gave 1.72 g of the target compound (93% of theory).

LC-MS (Method P): Rₜ=1.32 min; m/z=349 (M+H)⁺

¹H-NMR (300 MHz, DMSO-d₆): δ [ppm]=12.44 (hr. s, 1H), 8.75 (s, 1H), 7.60-7.52 (m, 1H), 7.25-7.17 (m, 2H), 7.11 (s, 1H), 5.29 (s, 2H), 3.93 (s, 3H), 2.34 (s, 3H).

### Example 73A

### ent-Benzyl {1-[({2-chloro-8-[(2,6-difluorobenzyl)oxy]-6-methylimidazo[1,2-a]pyridin-3-yl}carbonyl)amino]-5,5,5-trifluoro-2-methylpentan-2-yl}carbamate trifluoroacetate (enantiomer B)

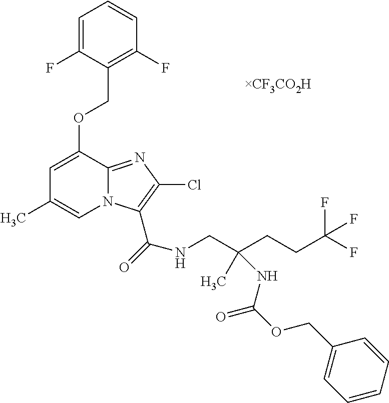 [SMILES: Cc1cc(OCc2c(F)cccc2F)c2nc(Cl)c(C(=O)NCC(C)(CCC(F)(F)F)NC(=O)OCc3ccccc3)n2c1]

50 mg (0.142 mmol) of 2-chloro-8-[(2,6-difluorobenzyl)oxy]-6-methylimidazo[1,2-a]pyridine-3-carboxylic acid from Example 71A were initially charged together with 70 mg (0.184 mmol) of HATU and 123 μl (0.709 mmol) of N,N-diisopropylethylamine in 0.47 ml of DMF, and the mixture was stirred at room temperature for 20 min. 59 mg (0.184 mmol; purity 95%) of ent-benzyl (1-amino-5,5,5-trifluoro-2-methylpentan-2-yl)carbamate (enantiomer B) from Example 37A were added to the reaction solution and the mixture was stirred at RT for 45 minutes, Acetonitrile, water and TFA were added and the reaction solution was purified by preparative HPLC (RP18 column, mobile phase: acetonitrile/water gradient with addition of 0.1% TFA). The product fractions were combined and concentrated. This gave 72 mg of the target compound (67% of theory).

LC-MS (Method D): Rₜ=1.30 min

MS (ESpos): m/z=639 (M-TFA+H)⁺

### Example 74A

### ent-Benzyl {1-[({8-[(2,6-difluorobenzyl)oxy]-2-methoxy-6-methylimidazo[1,2-a]pyridin-3-yl}carbonyl)amino]-2-methylbutan-2-yl}carbamate trifluoroacetate (enantiomer B)

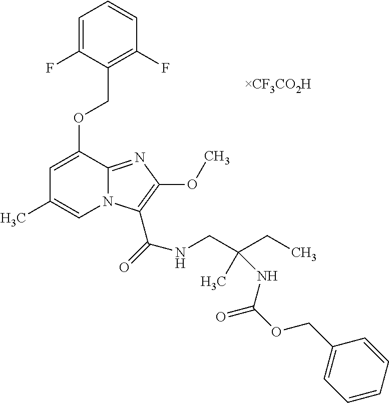 [SMILES: CCC(C)(CNC(=O)c1c(OC)nc2c(OCc3c(F)cccc3F)cc(C)cn12)NC(=O)OCc1ccccc1]

60 mg (0.064 mmol, purity 37%) of 8-[(2,6-difluorobenzyl)oxy]-2-methoxy-6-methylimidazo[1,2-a]pyridine-3-carboxylic acid from Example 72A were initially charged together with 29 mg (0.076 mmol) of HATU and 33 μl (0.191 mmol) of N,N-diisopropylethylamine in 0.40 ml of DMF, and the mixture was stirred at room temperature for 20 min. 38 mg (0.159 mmol) of ent-benzyl (1-amino-2-methylbutan-2-yl)carbamate (enantiomer B) [described in WO2014/068099, Example No. 275A] were added to the reaction solution and the mixture was stirred at 60° C. for 30 minutes. The reaction solution was purified by preparative HPLC (RP18 column, mobile phase: acetonitrile/water gradient with addition of 0.1% TFA). The product fractions were combined and concentrated. This gave 34 mg of the target compound (78% of theory).

LC-MS (Method D): Rₜ=1.33 min

MS (ESpos): m/z=567 (M-TFA+H)⁺

### Example 75A

### ent-Benzyl {1-[({8-[(2,6-difluorobenzyl)oxy]-2-methoxy-6-methylimidazo[1,2-a]pyridin-3-yl}carbonyl)amino]-5,5,5-trifluoro-2-methylpentan-2-yl}carbamate trifluoroacetate (enantiomer B)

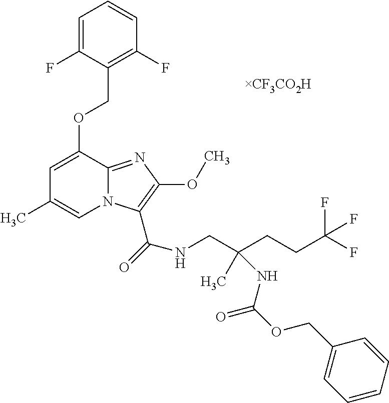 [SMILES: COc1nc2c(OCc3c(F)cccc3F)cc(C)cn2c1C(=O)NCC(C)(CCC(F)(F)F)NC(=O)OCc1ccccc1]

50 mg (0.144 mmol) of 8-[(2,6-difluorobenzyl)oxy]-2-methoxy-6-methylimidazo[1,2-a]pyridine-3-carboxylic acid from Example 72A were initially charged together with 71 mg (0.187 mmol) of HATU and 125 μl (0.718 mmol) of N,N-diisopropylethylamine in 0.48 ml of DMF, and the mixture was stirred at room temperature for 20 min. 60 mg (0.187 mmol; purity 95%) of ent-benzyl (1-amino-5,5,5-trifluoro-2-methylpentan-2-yl)carbamate (enantiomer B) from Example 37A were added to the reaction solution and the mixture was stirred at RT for 45 minutes, Acetonitrile, water and TFA were added and the reaction solution was purified by preparative HPLC (RP18 column, mobile phase: acetonitrile/water gradient with addition of 0.1% TFA). The product fractions were combined and concentrated. This gave 50 mg of the target compound (46% of theory).

LC-MS (Method D): Rₜ=1.31 min

MS (ESpos): m/z=635 (M-TFA+H)⁺

### WORKING EXAMPLES

### Example 1

### rac-N-(2-Amino-2-methylbutyl)-8-[(2,6-difluorobenzyl)oxy]-2-methyl-6-(pyridin-3-yl)imidazo[1,2-a]pyridine-3-carboxamide

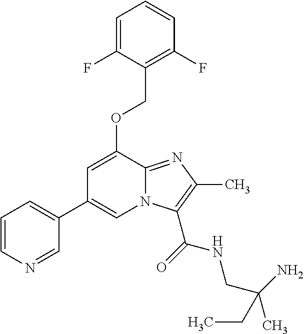 [SMILES: CCC(C)(N)CNC(=O)c1c(C)nc2c(OCc3c(F)cccc3F)cc(-c3cccnc3)cn12]

1 ml of trifluoroacetic acid was added to a solution of 90 mg (0.15 mmol) of rac-tert-butyl {1-[({8-[(2,6-difluorobenzyl)oxy]-2-methy-6-(pyridin-3-yl)imidazo[1,2-a]pyridin-3-yl}carbonyl)amino]-2-methylbutan-2-yl}carbamate (Example 6A) in 4 ml of dichloromethane. The reaction mixture was stirred at room temperature for 1 h and then concentrated under reduced pressure. The residue was purified by chromatography on SCX-2 silica gel (mobile phase: methanol then 20% (2M ammonia in methanol) in dichloromethane). This gave 51 mg of the target product (69% of theory).

LC-MS (Method B): Rₜ=2.47 min; m/z=480.2 (M+H)⁺

¹H-NMR (400 MHz, DMSO-ds): δ [ppm]=0.82 (t, 3H), 0.94 (s, 3H), 1.26-1.37 (m, 2H), 1.48 (s, 2H), 2.53 (s, 3H), 3.12 (d, 1H), 3.19 (d, 1H), 5.41 (s, 2H), 7.21 (t, 2H), 7.37 (d, 1H), 7.49 (ddd, 1H), 7.55 (ddd, 1H), 7.70 (s, 1H), 8.10 (ddd, 1H), 8.58 (dd, 1H), 8.90-8.93 (m, 2H).

### Example 2

### rac-N-(2-Amino-2-methylbutyl)-6-cyclopropyl-8-[(2,6-difluorobenzyl)oxy]-2-methylimidazo[1,2-a]pyridine-3-carboxamide hydrochloride

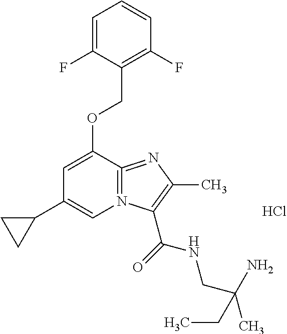

1 ml of trifluoroacetic acid were added to a solution of 56 mg (0.10 mmol) of rac-tert-butyl {1-[({6-cyclopropyl-8-[(2,6-difluorobenzyl)oxy]-2-methylimidazo [1,2-a]pyridin-3-yl}carbonyl)amino]-2-methylbutan-2-yl}carbamate (Example 7A) in 4 ml of dichloromethane. The reaction mixture was stirred at room temperature for 1 h and concentrated under reduced pressure. The residue was purified by chromatography on SCX-2 silica gel (mobile phase: methanol then 20% (2M ammonia in methanol) in dichloromethane). This gave the free base of the target product. This was taken up in acetonitrile (0.5 ml) and 0.1 N aqueous hydrochloric acid (2 ml) and lyophilized, giving 20 mg (40% of theory) of the target product.

LC-MS (Method B): Rₜ=2.71 min; m/z=443.2 (M+H)⁺

¹H-NMR (400 MHz, DMSO-d₆): δ [ppm]=0.81-0.85 (m, 2H), 0.90 (t, 3H), 0.98-1.03 (m, 2H), 1.22 (s, 3H), 1.58-1.68 (m, 2H), 2.05-2.12 (m, 1H), 2.58 (s, 3H), 3.36-3.50 (m, 2H), 5.43 (s, 2H), 7.17-7.25 (m, 3H), 7.52-7.60 (m, 1H), 8.08 (s, 3H), 8.52 (s, 1H), 8.87 (s, 1H).

### Example 3

### rac-N-(2-Amino-2-methylbutyl)-8-[(2,6-difluorobenzyl)oxy]-2-methyl-6-(1H-pyrazol-1-yl)imidazo[1,2-a]pyridine-3-carboxamide hydrochloride

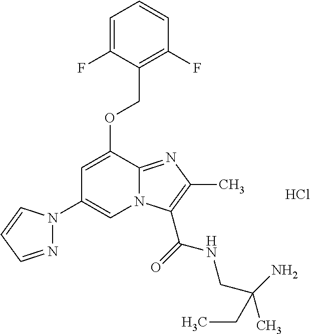 [SMILES: CCC(C)(N)CNC(=O)c1c(C)nc2c(OCc3c(F)cccc3F)cc(-n3cccn3)cn12 ; Cl]

1 ml of trifluoroacetic acid was added to a solution of 15 mg (0.10 mmol) of rac-tert-butyl {1-[({8-[(2,6-difluorobenzyl)oxy]-2-methyl-6-(1H-pyrazol-1-yl)imidazol[1,2-a]pyridin-3-yl}carbonyl)amino]-2-methylbutan-2-yl}carbamate (Example 8A) in 4 ml of dichloromethane. The reaction mixture was stirred at room temperature for 1 h and then concentrated under reduced pressure. The residue was purified by chromatography on SCX-2 silica gel (mobile phase: methanol then 20% (2M ammonia in methanol) in dichloromethane) and by preparative LC-MS (Method G). This gave the free base of the target product. This was taken up in acetonitrile (0.5 ml) and 0.1 N aqueous hydrochloric acid (2 ml) and lyophilized, giving 3 mg (23% of theory) of the target product.

LC-MS (Method B): Rₜ=2.92 min; m/z=469.2 (M+H)⁺

¹H-NMR (400 MHz, DMSO-d₆): δ [ppm]=0.91 (t, 3H), 1.22 (s, 3H), 1.58-1.70 (m, 2H), 2.59 (s, 3H), 3.39-3.60 (r, 2H), 5.47 (s, 2H), 6.60 (t, 1H), 7.23 (t, 2H), 7.58 (ddd, 1H), 7.79 (d, 1H), 7.82 (s, 1H), 7.97 (s, 3H), 8.39 (s, 1H), 8.61 (d, 1H).

### Example 4

### rac-N-(2-Amino-2-methylbutyl)-8-[(2,6-difluorobenzyl)oxy]-6-(methoxymethyl)-2-methylimidazo[1,2-a]pyridine-3-carboxamide hydrochloride

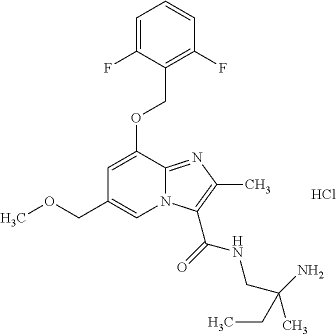 [SMILES: CCC(C)(N)CNC(=O)c1c(C)nc2c(OCc3c(F)cccc3F)cc(COC)cn12 ; Cl]

1 ml of trifluoroacetic acid were added to a solution of 60 mg (0.11 mmol) of rac-tert-butyl {1-[({8-[(2,6-di fluorobenzyl)oxy]-6-(methoxymethyl)-2-methylimidazo[1,2-a]pyridin-3-yl}carbonyl)amino]-2-methylbutan-2-yl}carbamate (Example 9A) in 4 ml of dichloromethane. The reaction mixture was stirred at room temperature for 1 h and concentrated under reduced pressure. The residue was purified by chromatography on SCX-2 silica gel (mobile phase: methanol then 20% (2M ammonia in methanol) in dichloromethane) and by preparative LC-MS (Method G). The residue was taken up in acetonitrile (0.5 ml) and 0.1 N aqueous hydrochloric acid (2 ml) and lyophilized, giving 28 mg of the target product (53% of theory).

LC-MS (Method B): Rₜ=2.50 min; m/z=447.2 (M+H)⁺

¹H-NMR (400 MHz, DMSO-d₆): δ [ppm]=0.91 (t, 3H), 1.21 (s, 3H), 1.63 (dd, 2H), 2.60 (s, 3H), 3.32 (s, 3H), 3.42-3.55 (m, 2H), 4.49 (s, 2H), 5.37 (s, 2H), 7.21 (t, 2H), 7.38 (s, 1H), 7.56 (ddd, 1H), 8.08 (s, 3H), 8.59 (s, 1H), 8.65 (s, 1H).

### Example 5

### rac-N-(2-Amino-2-methylbutyl)-8-[(2,6-difluorobenzyl)oxy]-6-(difluoromethoxy)-2-methylimidazo[1,2-a]pyridine-3-carboxamide

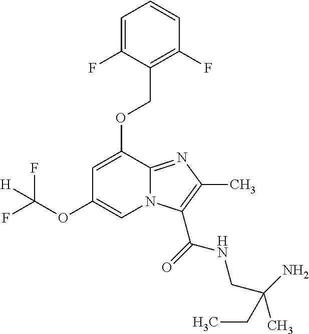 [SMILES: CCC(C)(N)CNC(=O)c1c(C)nc2c(OCc3c(F)cccc3F)cc(OC(F)F)cn12]

1 ml of trifluoroacetic acid were added to a solution of 13 mg (0.02 mmol) of rac-tert-butyl {1-[({8-[(2,6-difluorobenzyl)oxy]-6-(difluoromethoxy)-2-methylimidazo[1,2-a]pyridin-3-yl}carbonyl)amino]-2-methylbutan-2-yl}carbamate (Example 12A) in 4 ml of dichloromethane. The reaction mixture was stirred at room temperature for 1 h and concentrated under reduced pressure. The residue was purified by chromatography on SCX-2 silica gel (mobile phase: methanol then 20% (2M ammonia in methanol) in dichloromethane) and by preparative LC-MS (Method G). This gave 7 mg of the title compound (65% of theory).

LC-MS (Method B): Rₜ=3.05 min; m/z=469 (M+H)⁺

¹H-NMR (400 MHz, DMSO-d₆): δ [ppm]=0.82 (t, 3H), 0.92 (s, 3H), 1.24-1.40 (m, 4H), 2.51 (s, 3H), 3.16 (dd, 2H), 5.29 (s, 2H), 7.06 (d, 1H), 7.19 (t, 1H), 7.20 (t, 2H), 7.51-7.69 (m, 2H), 8.66 (d, 1H).

### Example 6

### rac-N-(2-Amino-2-methylbutyl)-8-[(2,6-difluorobenzyl)oxy]-2-methyl-6-(1-methyl-1H-pyrazol-4-yl)imidazo[1,2-a]pyridine-3-carboxamide

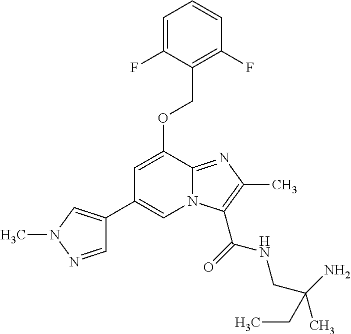 [SMILES: CCC(C)(N)CNC(=O)c1c(C)nc2c(OCc3c(F)cccc3F)cc(-c3cnn(C)c3)cn12]

1 ml of trifluoroacetic acid was added to a solution of 57 mg (0.10 mmol) of rac-tert-butyl {1-[({8-[(2,6-difluorobenzyl)oxy]-2-methyl-6-(1-methyl-1H-pyrazol-4-yl)imidazo[1,2-a]pyridin-3-yl}carbonyl)amino]-2-methylbutan-2-yl}carbamate (Example 41A) in 4 ml of dichloromethane. The reaction mixture was stirred at room temperature for 1 h and concentrated under reduced pressure. The residue was purified by chromatography on SCX-2 silica gel (mobile phase: methanol then 20% (2M ammonia in methanol) in dichloromethane). This gave 20 mg of the target product (40% of theory).

LC-MS (Method B): Rₜ=2.51 min; m/z=483 (M+H)⁺

¹H-NMR (400 MHz, DMSO-d₆): δ [ppm]=0.83 (t, 3H), 0.95 (s, 3H), 1.29-1.37 (m, 2H), 1.63-1.64 (m, 2H), 2.50 (s, 3H), 3.11-3.21 (m, 2H), 3.84 (s, 3H), 5.35 (s, 2H), 7.17-7.22 (m, 3H), 7.55 (ddd, 1H), 7.63 (s, 1H), 7.83 (s, 1H), 8.16 (s, 1H), 8.80 (d, 1H).

### Example 7

### rac-N-(2-Amino-2-methylbutyl)-8-[(2,6-difluorobenzyl)oxy]-2-methyl-6-(1,3-oxazol-5-yl)imidazo[1,2-a]pyridine-3-carboxamide

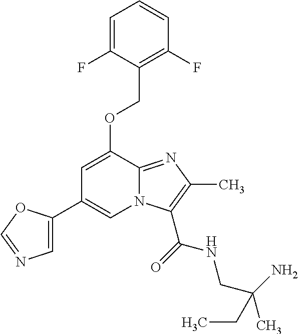 [SMILES: CCC(C)(N)CNC(=O)c1c(C)nc2c(OCc3c(F)cccc3F)cc(-c3cnco3)cn12]

1 ml of trifluoroacetic acid was added to a solution of 43 mg (0.08 mmol) of rac-tert-butyl {1-[({8-[(2,6-difluorobenzyl)oxy]-2-methyl-6-(1,3-oxazol-5-yl)imidazo[1,2-a]pyridin-3-yl}carbonyl)amino]-2-methylbutan-2-yl}carbamate (Example 13A) in 4 ml of dichloromethane. The reaction mixture was stirred at room temperature for 1 h and then concentrated under reduced pressure. The residue was purified by chromatography on SCX-2 silica gel (mobile phase: methanol then 20% (2M ammonia in methanol) in dichloromethane) and by preparative LC-MS (Method G). This gave 11 mg of the target product (31% of theory).

LC-MS (Method B): R=2.73 min; m/z=470 (M+H)⁺

¹H-NMR (400 MHz, DMSO-d): δ [ppm]=0.83 (t, 3H), 0.94 (s, 3H), 1.28-1.40 (m, 4H), 2.52 (s, 3H), 3.18 (dd, 2H), 5.37 (s, 2H), 7.21 (dd, 2H), 7.37 (d, 1H), 7.51-7.61 (m, 1H), 7.67 (s, 1H), 7.74 (s, 1H), 8.45 (s, 1H), 9.00 (d, 1H).

### Example 8

### rac-N-(2-Amino-2-methylbutyl)-8-[(2,6-difluorobenzyl)oxy]-2-(methoxymethyl)-6-methylimidazo[1,2-a]pyridine-3-carboxamide hydrochloride

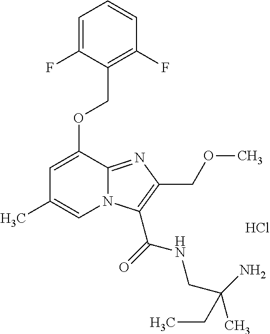 [SMILES: CCC(C)(N)CNC(=O)c1c(COC)nc2c(OCc3c(F)cccc3F)cc(C)cn12 ; Cl]

A solution of 258 mg (0.47 mmol) of rac-tert-butyl {1-[({8-[(2,6-difluorobenzyl)oxy]-2-(methoxymethyl)-6-methylimidazo[1,2-a]pyridin-3-yl}carbonyl)amino]-2-methylbutan-2-yl}carbamate (Example 16A) in 2 ml of trifluoroacetic acid and 8 ml of dichloromethane was stirred al room temperature for 18 h and then concentrated under reduced pressure. The residue was purified by chromatography on SCX-2 silica gel (mobile phase: methanol then 20% (2M ammonia in methanol) in dichloromethane) and by preparative LC-MS (Method G) and converted into the corresponding hydrochloride. This gave 150 mg of the target product (70% of theory).

LC-MS (Method B): Rₜ=2.43 min; m/z=447 (M+H)⁺

¹H-NMR (400 MHz, DMSO-d₆): δ [ppm]=0.82 (t, 3H), 1.13 (s, 3H), 1.55 (tdd, 2H), 2.37 (s, 3H), 3.19-3.28 (m, 2H), 3.73 (s, 2H), 4.02 (s, 3H), 5.35 (s, 2H), 7.22 (t, 2H), 7.58 (ddd, 1H), 8.06 (s, 4H), 8.53 (s, 1H).

### Example 9

### ent-N-(2-Amino-5,5,5-trifluoro-2-methylpentyl)-8-[(3-fluoropyridin-2-yl)methoxy]-2,6-dimethylimidazo[1,2-a]pyridine-3-carboxamide (enantiomer B)

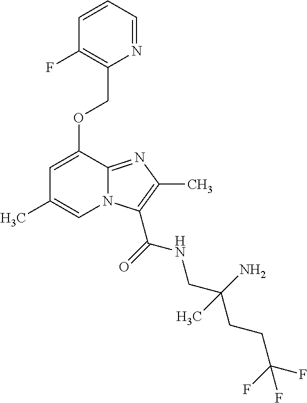 [SMILES: Cc1cc(OCc2ncccc2F)c2nc(C)c(C(=O)NCC(C)(N)CCC(F)(F)F)n2c1]

A mixture of 98 mg (0.14 mmol) of ent-benzyl {5,5,5-trifluoro-1-[({8-[(3-fluoropyridin-2-yl)methoxy]-2,6-dimethylimidazo[1,2-a]pyridin-3-yl}carbonyl)amino]-2-methylpentan-2-yl}carbamate trifluoroacetate (enantiomer B) from Example 38A and 4.4 mg of 10% palladium on activated carbon in 3.5 ml of ethanol was hydrogenated at room temperature and standard pressure for 45 min. Another 15 mg of 10% palladium on activated carbon were added, and the mixture was hydrogenated at room temperature and standard pressure for another 1 h. Subsequently, the mixture was filtered through a Millipore filter and washed with ethanol, and the filtrate was concentrated. The crude product was purified by preparative HPLC (RP-C18, mobile phase: acetonitrile/water gradient with addition of 0.1% TFA). The product fractions were taken up in dichloromethane and washed twice with saturated aqueous sodium bicarbonate solution. The combined aqueous phases were extracted twice with dichloromethane. The combined organic phases were dried over sodium sulphate, filtered and concentrated. This gave 18 mg of the title compound (28% of theory).

LC-MS (Method D): Rₜ=0.58 min

MS (ESpos): m/z=468 (M+H)⁺

¹H-NMR (500 MHz, DMSO-d₆): δ [ppm]=1.03 (s, 3H), 1.49-1.59 (m, 2H), 1.78 (br. s, 2H), 2.26-2.48 (m, 5H), 2.50 (s, 3H, overlapped by solvent peak), 3.18-3.32 (m, 2H), 5.39 (s, 2H), 6.89 (s, 1H), 7.57-7.61 (m, 1H), 7.74-7.88 (m, 2H), 8.40 (s, 1H), 8.50 (d, 1H).

### Example 10

### ent-N-(2-Amino-5,5,5-trifluoro-2-methylpentyl)-6-chloro-8-[(3-fluoropyridin-2-yl)methoxy]-2-methylimidazo[1,2-a]pyridine-3-carboxamide (enantiomer B)

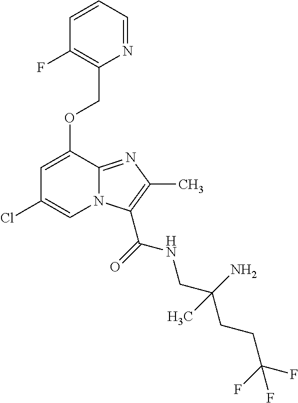 [SMILES: Cc1nc2c(OCc3ncccc3F)cc(Cl)cn2c1C(=O)NCC(C)(N)CCC(F)(F)F]

A mixture of 103 mg (0.14 mmol) of ent-benzyl {1-[({6-chloro-8-[(3-fluoropyridin-2-yl)methoxy]-2-methylimidazo [1,2-a]pyridin-3-yl}carbonyl)amino]-5,5,5-trifluoro-2-methylpentan-2-yl}carbamate trifluoroacetate (enantiomer B) from Example 39A and 4.4 mg of 10% palladium on activated carbon in 3.5 ml of ethanol was hydrogenated at room temperature and standard pressure for 45 min. Subsequently, the mixture was filtered through a Millipore filter and washed with ethanol, and the filtrate was concentrated. The crude product was purified by preparative HPLC (RP-C18, mobile phase: acetonitrile/water gradient with addition of 0.1% TFA). The product fractions were taken up in dichloromethane and a little methanol and washed twice with saturated aqueous sodium bicarbonate solution. The combined aqueous phases were extracted twice with dichloromethane. The combined organic phases were dried over sodium sulphate, filtered and concentrated by evaporation. This gave 16 mg of the title compound (23% of theory).

LC-MS (Method D): Rₜ=0.65 main

MS (ESpos): m/z=488 (M+H)⁺

¹H-NMR (500 MHz, DMSO-d₆): d [ppm]=1.02 (s, 3H), 1.48-1.57 (m, 2H), 1.63 (br. s, 2H), 2.27-2.47 (m, 2H), 2.50 (s, 3H; overlapped by solvent peak), 3.18-3.31 (m, 2H), 5.48 (s, 2H), 7.18 (s, 1H), 7.57-7.62 (m, 1H), 783-7.92 (m, 2H), 8.51 (d, 1H), 8.69 (s, 1H).

### Example 11

### ent-N-(2-Amino-5,5,5-trifluoro-2-methylpentyl)-8-[(3-fluoropyridin-2-yl)methoxy]-2-methylimidazo[1,2-a]pyridine-3-carboxamide (enantiomer B)

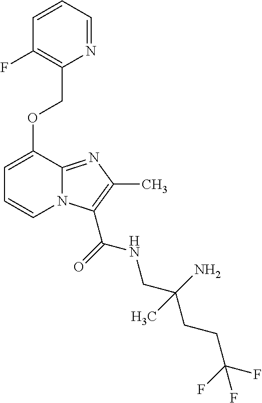 [SMILES: Cc1nc2c(OCc3ncccc3F)cccn2c1C(=O)NCC(C)(N)CCC(F)(F)F]

A mixture of 103 mg (0.14 mmol) of ent-benzyl {1-[({6-chloro-8-[(3-fluoropyridin-2-yl)methoxy]-2-methylimidazo [1,2-a]pyridin-3-yl}carbonyl)amino]-5,5,5-trifluoro-2-methylpentan-2-yl}carbamate trifluoroacetate (enantiomer B) from Example 39A and 4.4 mg of 10% palladium on activated carbon in 3.5 ml of ethanol was hydrogenated at room temperature and standard pressure for 45 min. Subsequently, the mixture was filtered through a Millipore filter and washed with ethanol, and the filtrate was concentrated. The crude product was purified by preparative HPLC (RP-C18, mobile phase: acetonitrile/water gradient with addition of 0.1% TFA). The product fractions were taken up in dichloromethane and a little methanol and washed twice with saturated aqueous sodium bicarbonate solution. The combined aqueous phases were extracted twice with dichloromethane. The combined organic phases were dried over sodium sulphate, filtered and concentrated by evaporation. This gave 11 mg of the title compound (17% of theory).

LC-MS (Method D): Rₜ=0.50 min

MS (ESpos): m/z=454 (M+H)⁺

¹H-NMR (500 MHz, DMSO-d₆): δ [ppm]=1.02 (s, 3H), 1.48-1.57 (m, 2H), 1.63 (br. s, 2H), 2.26-2.47 (m, 2H), 2.56 (s, 3H; partially overlapped by solvent peak), 3.19-3.31 (m, 2H), 5.42 (s, 2H), 6.90 (t, 1H), 6.99 (d, 1H), 7.56-7.62 (m, 1H), 7.77-7.87 (m, 2H), 8.48 (d, 1H), 8.58 (d, 1H).

### Example 12

### N-(3-Amino-2,2-difluoropropyl)-8-[(2,6-di fluorobenzyl)oxy]-2,6-dimethylimidazo [1,2-a]pyridine-3-carboxamide

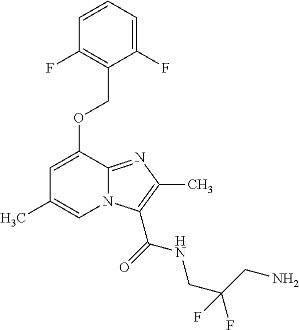 [SMILES: Cc1cc(OCc2c(F)cccc2F)c2nc(C)c(C(=O)NCC(F)(F)CN)n2c1]

163 mg (0.24 mmol, purity 93%) of tert-butyl {3-[({8-[(2,6-difluorobenzyl)oxy]-2,6-dimethylimidazo[1,2-a]pyridin-3-yl}carbonyl)amino]-2,2-difluoropropyl}carbamate trifluoroacetate from Example 40A were dissolved in 1.0 ml of diethyl ether, and 1.2 ml of a 2 N solution of hydrogen chloride in diethyl ether were added. The reaction mixture was stirred at room temperature overnight. The reaction mixture was concentrated, dissolved in acetonitrile/water, TFA was added and the product was purified by preparative HPLC (RP18 column, mobile phase: acetonitrile/water gradient with addition of 0.1% TFA). The concentrated product fractions were dissolved in dichloromethane and washed twice with saturated aqueous sodium bicarbonate solution. The combined aqueous phases were extracted twice with dichloromethane. The combined organic phases were dried over sodium sulphate, filtered and concentrated. This gave 96 mg of the target compound (94% of theory).

LC-MS (Method L): Rₜ=1.49 min

MS (ESpos): m/z=425 (M+H)⁺

¹H-NMR (600 MHz, DMSO-d₆): δ [ppm]=1.78 (br. s, 2H), 2.31 (s, 3H), 2.50 (s, 3H; overlapped by solvent peak), 2.91 (t, 2H), 3.79-3.86 (m, 2H), 5.29 (s, 2H), 6.94 (s, 1H), 7.20-7.25 (m, 2H), 7.56-7.62 (m, 1H), 8.19 (t, 1H), 8.40 (s, 1H).

### Example 13

### ent-N-[2-Amino-2-methyl-4-(trimethylsilyl)butyl]-8-[(2,6-difluorobenzyl)oxy]-2,6-dimethylimidazo[1,2-a]pyridine-3-carboxamide (enantiomer A)

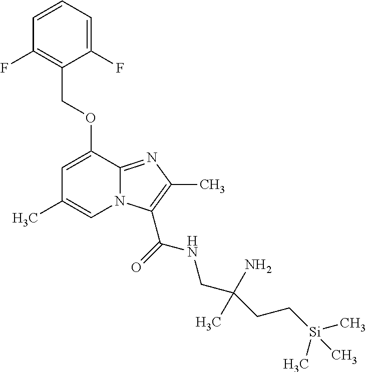 [SMILES: Cc1cc(OCc2c(F)cccc2F)c2nc(C)c(C(=O)NCC(C)(N)CC[Si](C)(C)C)n2c1]

151 mg (0.20 mmol, purity 97%) of ent-benzyl {1-[({8-[(2,6-difluorobenzyl)oxy]-2,6-dimethylimidazo [1,2-a]pyridin-3-yl}carbonyl)amino]-2-methyl-4-(trimethylsilyl)butan-2-yl}carbamate trifluoroacetate (enantiomer A) from Example 48A were dissolved in 5.2 ml of ethanol, and 77 μl (0.99 mmol) of TFA and 6.3 mg (0.006 mmol) of 10% palladium on activated carbon were added under argon and the mixture was hydrogenated at standard pressure for 2 hours. The reaction solution was filtered using a Millipore filter and washed with ethanol, and the filtrate was concentrated. The residue was taken up in acetonitrile, water and TFA and purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with saturated aqueous sodium hydrogencarbonate solution. The aqueous phase was re-extracted twice with dichloromethane. The combined organic phases were dried over sodium sulphate, filtered and concentrated. This gave 83 mg of the target compound (84% of theory).

LC-MS (Method D): Rₜ=0.78 min

MS (ESpos): m/z=489 (M+H)⁺

¹H-NMR (400 MHz, DMSO-d₆): δ=−0.04 (s, 9H), 0.45-0.56 (m, 2H), 0.97 (s, 3H), 1.26-1.34 (m, 2H), 1.42 (br. s, 2H), 2.30 (s, 3H), 3.15-3.30 (m, 2H), 5.29 (s, 2H), 6.91 (s, 1H), 7.18-7.28 (m, 2H), 7.54-7.64 (m, 2H), 8.47 (s, 1H), [further signal hidden under solvent signal].

### Example 14

### ent-N-[2-Amino-2-methyl-4-(trimethylsilyl)butyl]-8-[(2,6-difluorobenzyl)oxy]-2,6-dimethylimidazo[1,2-a]pyridine-3-carboxamide (enantiomer B)

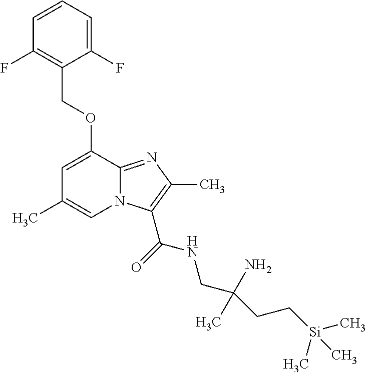 [SMILES: Cc1cc(OCc2c(F)cccc2F)c2nc(C)c(C(=O)NCC(C)(N)CC[Si](C)(C)C)n2c1]

168 mg (0.23 mmol, purity 99%) of ent-benzyl {1-[({8-[(2,6-difluorobenzyl)oxy]-2,6-dimethylimidazo[1,2-a]pyridin-3-yl}carbonyl)amino]-2-methyl-4-(trimethylsilyl)butan-2-yl}carbamate trifluoroacetate (enantiomer B) from Example 49A were dissolved in 5.9 ml of ethanol, and 87 μl (1.13 mmol) of TFA and 7.2 mg (0.007 mmol) of 10% palladium on activated carbon were added under argon and the mixture was hydrogenated at standard pressure for 2 hours. The reaction solution was filtered using a Millipore filter and washed with ethanol, and the filtrate was concentrated. The reaction solution was taken up in acetonitrile, water and TFA and purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with saturated aqueous sodium hydrogencarbonate solution. The combined aqueous phases were reextracted twice with dichloromethane. The combined organic phases were dried over sodium sulphate, filtered and concentrated. This gave 88 mg of the target compound (78% of theory).

LC-MS (Method D): Rₜ=0.75 min

MS (ESpos): m/z=489 (M+H)⁺

¹H-NMR (400 MHz, DMSO-d₆): δ=−0.04 (s, 9H), 0.45-0.58 (min, 2H), 0.98 (s, 3H), 1.25-1.39 (m, 2H), 1.90 (br. s, 2H), 2.30 (s, 3H), 3.17-3.30 (m, 2H), 5.29 (s, 2H), 6.91 (s, 1H), 7.19-7.27 (m, 2H), 7.54-7.64 (m, 2H), 8.47 (m, 1H), [further signal hidden under solvent signal].

### Example 15

### ent-N-[2-Amino-2-methyl-5-(trimethylsilyl)pentyl]-8-[(2,6-difluorobenzyl)oxy]-2,6-dimethylimidazo[1,2-a]pyridine-3-carboxamide (enantiomer A)

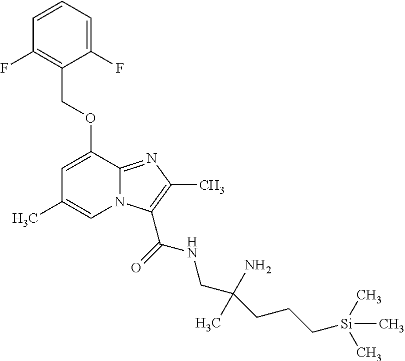 [SMILES: Cc1cc(OCc2c(F)cccc2F)c2nc(C)c(C(=O)NCC(C)(N)CCC[Si](C)(C)C)n2c1]

149 mg (0.20 mmol) of ent-benzyl {1-[({8-[(2,6-difluorobenzyl)oxy]-2,6-dimethylimidazo[1,2-a]pyridin-3-yl}carbonyl)amino]-2-methyl-5-(trimethylsilyl)pentan-2-yl}carbamate trifluoroacetate (enantiomer A) from Example 56A were dissolved in 6.7 ml of ethanol, and 76 μl (0.98 mmol) of TFA and 2 mg (0.002 mmol) of 10% palladium on activated carbon were added under argon and the mixture was hydrogenated at standard pressure for 2 hours. The reaction solution was filtered using a Millipore filter and washed with ethanol, and the filtrate was concentrated. The reaction solution was taken up in acetonitrile, water and TFA and purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with saturated aqueous sodium hydrogencarbonate solution. The combined aqueous phases were reextracted twice with dichloromethane. The combined organic phases were dried over sodium sulphate, filtered and concentrated. This gave 69 mg of the target compound (69% of theory).

LC-MS (Method O): Rₜ=1.41 min

MS (ESpos): m/z=503 (M+H)⁺

¹H-NMR (500 MHz, DMSO-d₆): δ=−0.03 (s, 9H), 0.41-0.48 (min, 2H), 0.99 (s, 3H), 1.29-1.42 (m, 4H), 1.53 (br. s, 2H), 2.30 (s, 3H), 3.16-3.24 (m, 2H), 5.29 (s, 2H), 6.91 (s, 1H), 7.19-7.27 (m, 2H), 7.53-7.63 (m, 2H), 8.48 (s, 1H), [further signal hidden under solvent signal].

### Example 16

### ent-N-[2-Amino-2-methyl-5-(trimethylsilyl)pentyl]-8-[(2,6-difluorobenzyl)oxy]-2,6-dimethylimidazo[1,2-a]pyridine-3-carboxamide (enantiomer B)

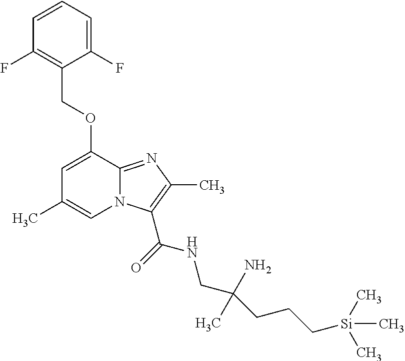 [SMILES: Cc1cc(OCc2c(F)cccc2F)c2nc(C)c(C(=O)NCC(C)(N)CCC[Si](C)(C)C)n2c1]

189 mg (0.25 mmol) of ent-benzyl {1-[({8-[(2,6-difluorobenzyl)oxy]-2,6-dimethylimidazo[1,2-a]pyridin-3-yl}carbonyl)amino]-2-methyl-5-(trimethylsilyl)pentan-2-yl}carbamate trifluoroacetate (enantiomer B) from Example 57A were dissolved in 8.5 ml of ethanol, and 96 μl (1.25 mmol) of TFA and 2.7 mg (0.002 mmol) of 10% palladium on activated carbon were added under argon and the mixture was hydrogenated at standard pressure for 2 hours. The reaction solution was filtered using a Millipore filter and washed with ethanol, and the filtrate was concentrated. The reaction solution was taken up in acetonitrile, water and TFA and purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with saturated aqueous sodium hydrogencarbonate solution. The combined aqueous phases were reextracted twice with dichloromethane. The combined organic phases were dried over sodium sulphate, filtered and concentrated. This gave 93 mg of the target compound (74% of theory).

LC-MS (Method D): Rₜ=0.84 min

MS (ESpos): m/z=503 (M+H)⁺

¹H-NMR (500 MHz, DMSO-d₆): δ=−0.03 (s, 9H), 0.41-0.48 (m, 2H), 0.99 (s, 3H), 1.29-1.42 (m, 4H), 1.48 (br. s, 2H), 2.31 (s, 3H), 3.16-3.23 (m, 2H), 5.29 (s, 2H), 6.91 (s, 1H), 7.19-7.27 (m, 2H), 7.54-7.63 (m, 2H), 8.48 (s, 1H), [further signal hidden under solvent signal].

### Example 17

### ent-N-(2-Amino-5,5,5-trifluoro-2-methylpentyl)-8-[(2,6-difluorobenzyl)oxy]-2-(3,3-difluorocyclobutyl)-6-methylimidazo[1,2-a]pyridine-3-carboxamide (enantiomer B)

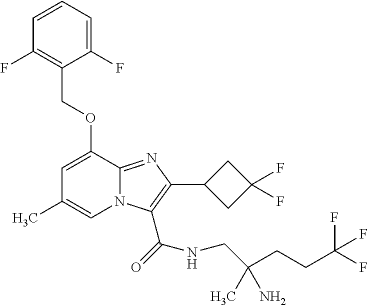 [SMILES: Cc1cc(OCc2c(F)cccc2F)c2nc(C3CC(F)(F)C3)c(C(=O)NCC(C)(N)CCC(F)(F)F)n2c1]

48 mg (0.059 mmol) of ent-benzyl {1-[({8-[(2,6-difluorobenzyl)oxy]-2-(3,3-difluorocyclobutyl)-6-methylimidazo[1,2-a]pyridin-3-yl}carbonyl)amino]-5,5,5-trifluoro-2-methylpentan-2-yl}carbamate trifluoroacetate (enantiomer B) from Example 64A were dissolved in 7 ml of ethanol, and 14 μl (0.178 mmol) of TFA and 2 mg (0.002 mmol) of 10% palladium on activated carbon were added under argon and the mixture was hydrogenated at standard pressure for 2 hours. The reaction solution was filtered through Celite and the filtrate was concentrated. The residue was dissolved in dichloromethane and washed twice with saturated aqueous sodium bicarbonate solution. The combined aqueous phases were reextracted twice with dichloromethane. The combined organic phases were dried over sodium sulphate, filtered, concentrated and lyophilized. This gave 29 mg of the target compound (87% of theory).

LC-MS (Method D): Rₜ=0.83 min

MS (ESpos): m/z=561 (M+H)⁺

¹H-NMR (400 MHz, DMSO-d₆): δ=1.06 (s, 3H), 1.53-1.62 (m, 2H), 2.31 (s, 3H), 2.32-2.46 (m, 2H), 2.87-3.01 (m, 5H), 3.76-3.87 (m, 1H), 5.34 (s, 2H), 6.97 (s, 1H), 7.20-7.28 (m, 2H), 7.54-7.64 (m, 1H), 7.99 (t, 1H), 8.30 (s, 1H), [further signal hidden under solvent signal].

### Example 18

### ent-N-(2-Amino-5,5,5-trifluoro-2-methylpentyl)-8-[(2,6-difluorobenzyl)oxy]-2-isopropyl-6-methylimidazo[1,2-a]pyridine-3-carboxamide (enantiomer B)

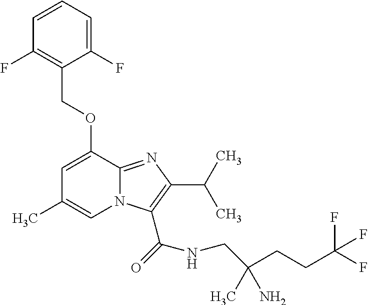 [SMILES: Cc1cc(OCc2c(F)cccc2F)c2nc(C(C)C)c(C(=O)NCC(C)(N)CCC(F)(F)F)n2c1]

37 mg (0.048 mmol) of ent-benzyl {i-[({8-[(2,6-difluorobenzyl)oxy]-2-isopropyl-6-methylmidazo[1,2-a]pyridin-3-yl}carbonyl)amino]-5,5,5-trifluoro-2-methylpentan-2-yl}carbamate trifluoroacetate (enantiomer B) from Example 67A were dissolved in 5 ml of ethanol, and 13 μl (0.172 mmol) of TFA and 2 mg (0.002 mmol) of 10% palladium on activated carbon were added under argon and the mixture was hydrogenated at standard pressure for 2 hours. The reaction solution was filtered through Celite and the filtrate was concentrated on a rotary evaporator. The residue was dissolved in dichloromethane and washed twice with saturated aqueous sodium bicarbonate solution. The combined aqueous phases were reextracted twice with dichloromethane. The combined organic phases were dried over sodium sulphate, filtered, concentrated and lyophilized. The residue was dissolved in dichloromethane and stirred with aqueous sodium bicarbonate solution overnight. The organic phase was then dried over sodium sulphate, filtered, concentrated and lyophilized. The residue was purified by preparative HPLC (column: XBridge C18 5 μm 75×30 mm, mobile phase: water, acetonitrile, acetonitrile/water 80/20+1% ammonia solution). This gave 8 mg (33% of theory) of the target compound.

LC-MS (Method K): Rₜ=2.63 min

MS (ESpos): m/z=513 (M+H)⁺

¹H-NMR (400 MHz, DMSO-d₆): δ=1.03 (s, 3H), 1.20-1.27 (m, 6H), 1.48-1.57 (m, 2H), 1.68 (s br., 2H), 2.29 (s, 3H), 2.32-2.47 (nm, 2H), 3.18-3.29 (m, 2H), 3.39-3.48 (m, 1H), 5.30 (s, 2H), 6.89 (s, 1H), 7.20-7.28 (m, 2H), 7.54-7.64 (m, 1H), 7.92 (t, 1H), 8.22 (s, 1H).

### Example 19

### ent-N-(2-Amino-5,5,5-trifluoro-2-methylpentyl)-2-chloro-8-[(2,6-difluorobenzyl)oxy]-6-methylimidazo[1,2-a]pyridine-3-carboxamide (enantiomer B)

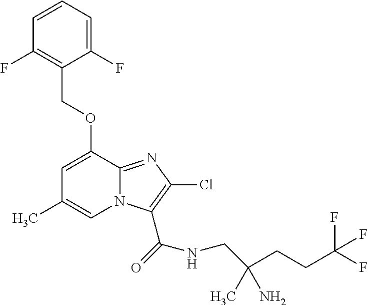 [SMILES: Cc1cc(OCc2c(F)cccc2F)c2nc(Cl)c(C(=O)NCC(C)(N)CCC(F)(F)F)n2c1]

67 mg (0.089 mmol) of ent-benzyl {1-[({2-chloro-8-[(2,6-difluorobenzyl)oxy]-6-methylmidazo[1,2-a]pyridin-3-yl}carbonyl)amino]-5,5,5-trifluoro-2-methylpentan-2-yl}carbamate trifluoroacetate (enantiomer B) from Example 73A were dissolved in 5 ml of TFA, and the mixture was stirred at room temperature for 4 days. The reaction solution was purified by preparative HPLC (RP18 column, mobile phase: acetonitrile/water gradient with addition of 0.1% TFA). The product fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulphate, filtered and concentrated. This gave 39 mg (86% of theory) of the target compound.

LC-MS (Method D): Rₜ=0.76 min

MS (ESpos): m/z=505 (M+H)⁺

¹H-NMR (400 MHz, DMSO-d₆): δ=1.03 (s, 3H), 1.51-1.60 (m, 2H), 1.82 (br. s, 2H), 2.23-2.47 (m, 5H), 3.18-3.29 (m, 2H), 5.31 (s, 2H), 7.11 (s, 1H), 7.19-7.28 (m, 2H), 7.54-7.64 (m, 1H), 7.95 (t, 1H), 8.60 (s, 1H).

### Example 20

### rac-N-(2-Amino-2-methylbutyl)-2-chloro-8-[(2,6-dichlorobenzyl)oxy]-6-methylimidazo[1,2-a]pyridine-3-carboxamide

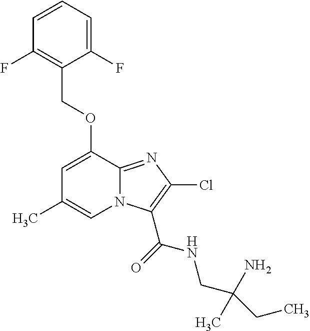 [SMILES: CCC(C)(N)CNC(=O)c1c(Cl)nc2c(OCc3c(F)cccc3F)cc(C)cn12]

40 mg (0.11 mmol) of 2-chloro-8-[(2,6-difluorobenzyl)oxy]-6-methylimidazo[1,2-a]pyridine-3-carboxylic acid from Example 71A were initially charged together with 47 mg (0.13 mmol) of HATU and 59 ii (0.34 mmol) of N,N-diisopropylethylamine in 0.4 ml of DMF, and the mixture was stirred at room temperature for 10 min. 13 mg (0.13 mmol) of rac-2-methylbutane-1,2-diamine were then added to the reaction solution and the mixture was stirred at RT for 4.5 h. The mixture was then diluted with acetonitrile and water, TFA was added and the mixture was purified by preparative HPLC (RP18 column, mobile phase: acetonitrile/water gradient with addition of 0.1% TFA). The product fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulphate, filtered and concentrated. This gave 31 ing of the target compound (61% of theory).

LC-MS (Method O): Rₜ=1.27 min

MS (ESpos): m/z=437 (M+H)⁺

¹H-NMR (500 MHz, DMSO-d₆): δ=0.86 (t, 3H), 0.98 (s, 3H), 1.31-1.42 (m, 2H), 1.49 (br. s, 2H), 2.35 (s, 3H), 3.15-3.27 (m, 2H), 5.32 (s, 2H), 7.12 (s, 1H), 7.19-7.28 (m, 2H), 7.55-7.64 (m, 1H), 7.80 (br. s, 1H), 8.72 (s, 1H).

### Example 21

### ent-N-(2-Amino-2-methylbutyl)-2-chloro-8-[(2,6-difluorobenzyl)oxy]-6-methylimidazo[1,2-a]pyridine-3-carboxamide (enantiomer A)

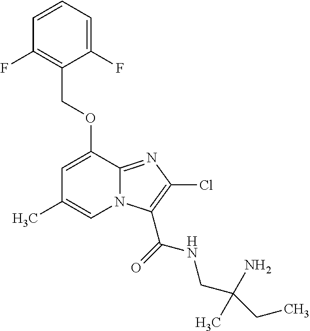 [SMILES: CCC(C)(N)CNC(=O)c1c(Cl)nc2c(OCc3c(F)cccc3F)cc(C)cn12]

25 mg of rac-N-(2-amino-2-methylbutyl)-2-chloro-8-[(2,6-difluorobenzyl)oxy]-6-methylimidazo[1,2-a]pyridine-3-carboxamide from Example 20 were separated into the enantiomers by preparative separation on a chiral phase [column: Daicel Chiralpak IF, 5 μm, 250×20 mm, mobile phase: 35% isohexane, 65% ethanol+0.2% diethylamine, flow rate: 15 ml/min, temperature: 40° C., detection: 220 nm]. The product was collected on dry ice and concentrated on a rotary evaporator.

Enantiomer A: 9 mg (>99% ee)

Rₜ=6.10 min [Daicel Chiralpak AZ-H, 250×4.6 mm, 5 μm, mobile phase: 30% isohexane, 70% ethanol+0.2% diethylamine, flow rate: 1 ml/min, temperature: 40° C., detection: 220 nm].

### Example 22

### ent-N-(2-Amino-2-methylbutyl)-2-chloro-8-[(2,6-difluorobenzyl)oxy]-6-methylimidazo[1,2-a]pyridine-3-carboxamide (enantiomer B)

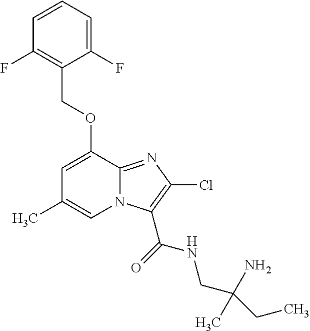 [SMILES: CCC(C)(N)CNC(=O)c1c(Cl)nc2c(OCc3c(F)cccc3F)cc(C)cn12]

25 mg of rac-N-(2-amino-2-methylbutyl)-2-chloro-8-[(2,6-difluorobenzyl)oxy]-6-methylimidazo[1,2-a]pyridine-3-carboxamide from Example 20 were separated into the enantiomers by preparative separation on a chiral phase [column: Daicel Chiralpak IF, 5 μm, 250×20 mm, mobile phase: 35% isohexane, 65% ethanol+0.2% diethylamine, flow rate: 15 ml/min, temperature: 40° C., detection: 220 nm]. The product was collected on dry ice and concentrated on a rotary evaporator.

enantiomer B: 11 mg (94% ee)

Rₜ=7.33 min [Daicel Chiralpak AZ-H, 250×4.6 mm, 5 μm, mobile phase: 30% isohexane, 70% ethanol+0.2% diethylamine, flow rate: 1 ml/min, temperature: 40° C., detection: 220 nm].

### Example 23

### rac-N-(2-Amino-2-methylpentyl)-2-chloro-8-[(2,6-difluorobenzyl)oxy]-6-methylmidazo[1,2-a]pyridine-3-carboxamide

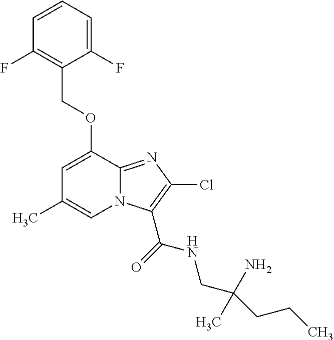 [SMILES: CCCC(C)(N)CNC(=O)c1c(Cl)nc2c(OCc3c(F)cccc3F)cc(C)cn12]

75 mg (0.21 mmol) of 2-chloro-8-[(2,6-difluorobenzyl)oxy]-6-methylimidazo[1,2-a]pyridine-3-carboxylic acid from Example 71A were initially charged together with 89 mg (0.23 mmol) of HATU and 111 μl (0.64 mmol) of N,N-diisopropylethylamine in 0.7 ml of DMF, and the mixture was stirred at room temperature for 10 min. 27 mg (0.23 mmol) of rac-2-methylpentane-1,2-diamine were then added to the reaction solution and the mixture was stirred at RT for 4.5 h. The mixture was then diluted with acetonitrile and water, TFA was added and the mixture was purified by preparative HPLC (RP18 column, mobile phase: acetonitrile/water gradient with addition of 0.1% TFA), The product fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulphate, filtered and concentrated. This gave 36 mg of the target compound (37% of theory).

LC-MS (Method D): Rₜ=0.76 min

MS (ESpos): m/z=451 (M+H)⁺

¹H-NMR (500 MHz, DMSO-d₆): δ=0.82-0.90 (m, 3H), 1.00 (s, 3H), 1.26-1.39 (m, 4H), 1.55 (br. s, 2H), 2.35 (s, 3H), 3.14-3.26 (m, 2H), 5.32 (s, 2H), 7.11 (s, 1H), 7.20-7.27 (m, 2H), 7.55-7.64 (m, 1H), 7.81 (br. s, 1H), 8.71 (s, 1H).

### Example 24

### ent-N-(2-Amino-2-methylpentyl)-2-chloro-8-[(2,6-difluorobenzyl)oxy]-6-methylimidazo[1,2-a]pyridine-3-carboxamide (enantiomer A)

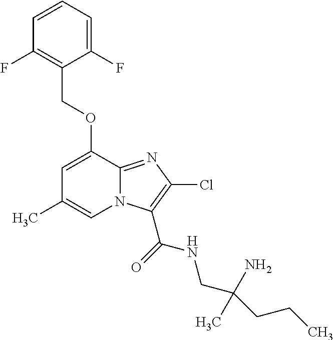 [SMILES: CCCC(C)(N)CNC(=O)c1c(Cl)nc2c(OCc3c(F)cccc3F)cc(C)cn12]

32 mg of rac-N-(2-amino-2-methylpentyl)-2-chloro-8-[(2,6-difluorobenzyl)oxy]-6-methylimidazo[1,2-a]pyridine-3-carboxamide from Example 23 were separated into the enantiomers by preparative separation on a chiral phase [column: Daicel Chiralpak IF, 5 μm, 250×20 mm, mobile phase: 50% isohexane, 50% ethanol+0.2% diethylamine, flow rate: 15 ml/min, temperature: 40° C., detection: 220 nm]. The product was collected on dry ice and concentrated on a rotary evaporator.

enantiomer A: 15 mg (>99% ee)

Rₜ=6.77 min [Daicel Chiralpak AZ-H, 250×4.6 mm, 5 μm, mobile phase: 50% isohexane, 50% ethanol+0.2% diethylamine, flow rate: 1 ml/min, temperature: 40° C., detection: 220 nm].

### Example 25

### ent-N-(2-Amino-2-methylpentyl)-2-chloro-8-[(2,6-difluorobenzyl)oxy]-6-methylimidazo[1,2-a]pyridine-3-carboxamide (enantiomer B)

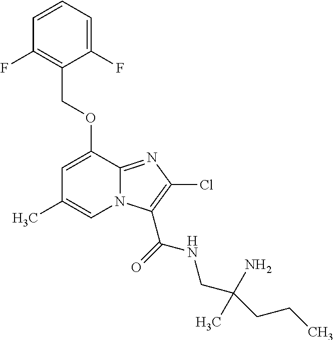 [SMILES: CCCC(C)(N)CNC(=O)c1c(Cl)nc2c(OCc3c(F)cccc3F)cc(C)cn12]

32 mg of rac-N-(2-amino-2-methylpentyl)-2-chloro-8-[(2,6-difluorobenzyl)oxy]-6-methylimidazo[1,2-a]pyridine-3-carboxamide from Example 23 were separated into the enantiomers by preparative separation on a chiral phase [column: Daicel Chiralpak IF, 5 μm, 250×20 mm, mobile phase: 50% isohexane, 50% ethanol+0.2% diethylamine, flow rate: 15 ml/min, temperature: 40° C., detection: 220 nm]. The product was collected on dry ice and concentrated on a rotary evaporator.

enantiomer B: 15 mg (98.8% ee)

Rₜ=9.05 min [Daicel Chiralpak AZ-H, 250×4.6 mm, 5 μm, mobile phase: 50% isohexane, 50% ethanol+0.2% diethylamine, flow rate: 1 ml/min, temperature: 40° C., detection: 220 nm].

### Example 26

### ent-N-(2-Amino-2-methylbutyl)-8-[(2,6-difluorobenzyl)oxy]-2-methoxy-6-methylimidazo[1,2-a]pyridine-3-carboxamide (enantiomer B)

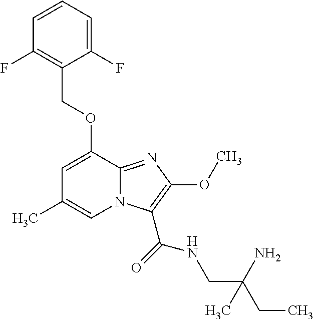 [SMILES: CCC(C)(N)CNC(=O)c1c(OC)nc2c(OCc3c(F)cccc3F)cc(C)cn12]

50 mg (0.073 mmol) of ent-benzyl {-[({8-[(2,6-difluorobenzyl)oxy]-2-methoxy-6-methylimidazo [1,2-a]pyridin-3-yl}carbonyl)amino]-2-methylbutan-2-yl}carbamate trifluoroacetate (enantiomer B) from Example 74A were dissolved in 2.5 ml of ethanol, 0.8 mg (0.001 mmol) of 10% palladium on activated carbon were added under argon and the mixture was hydrogenated at standard pressure for 3.5 hours. The reaction solution was filtered through a Millipore filter and the filtrate was concentrated. The residue was dissolved in 2.5 ml of ethanol, and 28 μl (0.367 mmol) of TFA and 0.8 mg (0.001 mmol) of 10% palladium on activated carbon were added under argon and the mixture was hydrogenated at standard pressure for 1.5 hours. The reaction solution was filtered through a Millipore filter and the filtrate was concentrated. A solution of ammonia in methanol was added to the residue and the product was purified by thick-layer chromatography (mobile phase: dichloromethane/2N ammonia in methanol 20/1.5). This gave 24 mg (72% of theory) of the target compound.

LC-MS (Method D): Rₜ=0.74 min

MS (ESpos): m/z=433 (M+H)⁺

¹H-NMR (400 MHz, DMSO-d₆): δ=0.84 (t, 3H), 0.94 (s, 3H), 1.23-1.38 (m, 2H), 1.42 (br. s, 2H), 2.34 (s, 3H), 3.10-3.23 (m, 2H), 4.05 (s, 3H), 5.30 (s, 2H), 7.07 (s, 1H), 7.21-7.30 (m, 3H), 7.54-7.64 (m, 1H), 9.02 (s, 1H).

### Example 27

### ent-N-(2-Amino-5,5,5-trifluoro-2-methylpentyl)-8-[(2,6-difluorobenzyl)oxy]-2-methoxy-6-methylimidazo[1,2-a]pyridine-3-carboxamide (enantiomer B)

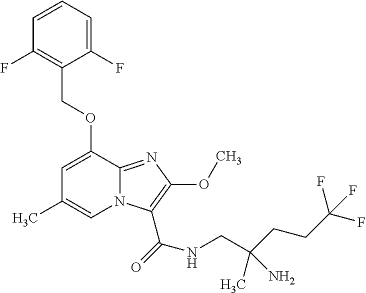 [SMILES: COc1nc2c(OCc3c(F)cccc3F)cc(C)cn2c1C(=O)NCC(C)(N)CCC(F)(F)F]

49 mg (0.065 mmol) of ent-benzyl {-[({8-[(2,6-difluorobenzyl)oxy]-2-methoxy-6-methylimidazo[1,2-a]pyridin-3-yl}carbonyl)amino]-5,5,5-tri fluoro-2-methylpentan-2-yl}carbamate trifluoroacetate (enantiomer B) from Example 75A were dissolved in 7 ml of ethanol, and 25 μl (0.327 mmol) of TFA and 2.1 mg (0.002 mmol) of 10% palladium on activated carbon were added under argon and the mixture was hydrogenated at standard pressure for 1 hour. The reaction solution was filtered through a Millipore filter and washed through with ethanol, and the filtrate was concentrated under reduced pressure. Acetonitrile, water and TFA were added to the residue and the product was purified by preparative HPLC (RP18 column, mobile phase: acetonitrile/water gradient with addition of 0.1% TFA). The product fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulphate, filtered and concentrated. This gave 31 mg of the target compound (91% of theory).

LC-MS (Method D): Rₜ=0.82 min

MS (ESpos): m/z=501 (M+H)⁺

¹H-NMR (400 MHz, DMSO-d₆): δ=0.99 (s, 3H), 1.51 (t, 2H), 1.92 (br. s, 2H), 2.24-2.44 (m, 5H), 3.14-3.29 (m, 2H), 4.05 (s, 3H), 5.31 (s, 2H), 7.08 (s, 1H), 7.19-7.30 (m, 3H), 7.54-7.64 (m, 1H), 8.98 (s, 1H).

### Example 28

### ent-2-Chloro-8-[(2,6-difluorobenzyl)oxy]-6-methyl-N-(6,6,7,7,7-pentafluoro-2-hydroxy-2-methylheptan-3-yl)imidazo[1,2-a]pyridine-3-carboxamide (enantiomer A)

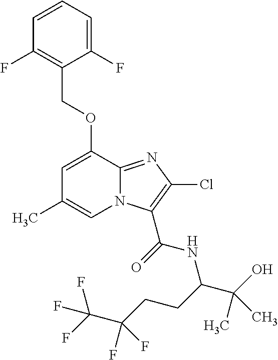 [SMILES: Cc1cc(OCc2c(F)cccc2F)c2nc(Cl)c(C(=O)NC(CCC(F)(F)C(F)(F)F)C(C)(C)O)n2c1]

40 mg (0.113 mmol) of 2-chloro-8-[(2,6-difluorobenzyl)oxy]-6-methylimidazo[1,2-a]pyridine-3-carboxylic acid from Example 71A were initially charged together with 47 mg (0.125 mmol) of HATU and 99 μl (0.567 mmol) of N,N-diisopropylethylamine in 0.4 ml of DMF, and the mixture was stirred at room temperature for 10 min. 34 mg (0.125 mmol) of ent-3-amino-6,6,7,7,7-pentafluoro-2-methylheptan-2-ol hydrochloride (enantiomer A) [described in WO2014/068104, Example No. 138A] were then added to the reaction solution and the mixture was stirred at RT for 4.5 hours. Acetonitrile, water and TFA were added and the reaction solution was purified by preparative HPLC (RP18 column, mobile phase: acetonitrile/water gradient with addition of 0.1% TFA). The product fraction was concentrated, and the residue was dissolved in dichloromethane and washed twice with saturated aqueous sodium bicarbonate solution. The combined aqueous phases were reextracted twice with dichloromethane. The combined organic phases were dried over sodium sulphate, filtered, concentrated and lyophilized. This gave 47 mg of the target compound (72% of theory).

LC-MS (Method O): Rₜ=2.30 min

MS (ESpos): m/z=570 (M+H)⁺

¹H-NMR (500 MHz, DMSO-d₆): δ=1.14 (s, 3H), 1.21 (s, 3H), 1.67-1.77 (m, 1H), 2.00-2.10 (m, 1H), 2.12-2.31 (m, 2H), 2.35 (s, 3H), 3.97-4.04 (m, 1H), 4.75 (s, 1H), 5.32 (s, 2H), 7.12 (s, 1H), 7.20-7.27 (m, 2H), 7.55-7.63 (m, 1H), 7.69 (d, 1H), 8.55 (s, 1H).

### Example 29

### 8-[(2,6-Difluorobenzyl)oxy]-N-[(2R)-1-hydroxyhexan-2-yl]-2-methoxy-6-methylmidazo[1,2-a]pyridine-3-carboxamide

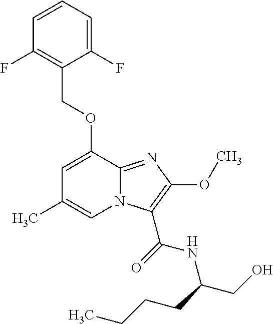 [SMILES: CCCC[C@H](CO)NC(=O)c1c(OC)nc2c(OCc3c(F)cccc3F)cc(C)cn12]

50 mg (0.144 mmol) of 8-[(2,6-difluorobenzyl)oxy]-2-methoxy-6-methylimidazo[1,2-a]pyridine-3-carboxylic acid from Example 72A were initially charged together with 71 mg (0.187 mmol) of HATU and 125 μl (0.718 mmol) of N,N-diisopropylethylamine in 0.50 ml of DMF, and the mixture was stirred at room temperature for 20 min. 22 mg (0.187 mmol) of (2R)-2-aminohexan-1-ol were then added to the reaction solution and the mixture was stirred at RT for 2 hours. Acetonitrile, water and TFA were added and the reaction solution was purified by preparative HPLC (RP18 column, mobile phase: acetonitrile/water gradient with addition of 0.1% TFA). The product fraction was concentrated, and the residue was dissolved in dichloromethane and washed twice with saturated aqueous sodium bicarbonate solution. The combined aqueous phases were reextracted twice with dichloromethane. The combined organic phases were dried over sodium sulphate, filtered, concentrated and lyophilized. This gave 22 mg of the target compound (33% of theory).

LC-MS (Method D): Rₜ=1.14 min

MS (ESpos): m/z=448 (M+H)⁺

¹H-NMR (400 MHz, DMSO-d₆): δ=0.86 (t, 3H), 1.22-1.35 (m, 4H), 1.40-1.52 (m, 1H), 1.54-1.65 (m, 1H), 2.34 (s, 3H), 3.38-3.53 (m, 2H), 3.90-4.00 (m, 1H), 4.05 (s, 3H), 4.82 (t, 1H), 5.30 (s, 2H), 6.91 (d, 1H), 7.08 (s, 1H), 7.20-7.28 (m, 2H), 7.54-7.64 (m, 1H), 9.02 (s, 1H).

### B. ASSESSMENT OF PHARMACOLOGICAL EFFICACY

The following abbreviations are used:

ATP adenosine triphosphate
Brij35 polyoxyethylene(23) lauryl ether
BSA bovine serum albumin
DTT dithiothreitol
TEA triethanolamine

The pharmacologicai action of the compounds of the invention can be demonstrated in the following assays:

### B-1. Measurement of sGC Enzyme Activity by Means of PPi Detection

Soluble guanylyl cyclase (sGC) converts GTP to cGMP and pyrophosphate (PPi) when stimulated. PPi is detected with the aid of the method described in WO 2008/061626. The signal that arises in the assay increases as the reaction progresses and serves as a measure of the sGC enzyme activity. With the aid of a PPi reference curve, the enzyme can be characterized in a known manner, for example in terms of conversion rate, stimulability or Michaelis constant

### Practice of the Test

To conduct the test, 29 μl of enzyme solution (0-10 nM soluble guanylyl cyclase (prepared according to Hönicka et al., Journal of Molecular Medicine 77(1999) 14-23), in 50 mM TEA, 2 mM magnesium chloride, 0.1% BSA (fraction V), 0.005% Brij 35, pH 7.5) were initially charged in the microplate, and 1 μl of the stimulator solution (0-10 μM 3-morpholinosydnonimine, SIN-1, Merck in DMSO) was added. The microplate was incubated at RT for 10 min. Then 20 μl of detection mix (1.2 nM Firefly Luciferase (Photinus pyralis luciferase, Promega), 29 μM dehydroluciferin (prepared according to Bitler & McElroy, Arch. Biochem. Biophys. 72 (1957) 358), 122 μM luciferin (Promega), 153 μM ATP (Sigma) and 0.4 mM DTT (Sigma) in 50 mM TEA, 2 mM magnesium chloride, 0.1% BSA (fi-action V), 0.005% Brij 35, pH 7.5) were added. The enzyme reaction was started by adding 20 μl of substrate solution (1.25 mM guanosine 5′-triphosphate (Sigma) in 50 mM TEA, 2 mM magnesium chloride, 0.1% BSA (fraction V), 0.005% Brij, pH 7.5) and analysed continuously in a luminometer.

### B-2. Effect on a Recombinant Guanylate Cyclase Reporter Cell Line

The cellular activity of the compounds of the invention is determined using a recombinant guanylate cyclase reporter cell line, as described in F. Wunder et al., Anal Biochem. 339, 104-112 (2005).

Representative MEC values (MEC=minimum effective concentration) for the compounds of the invention are shown in the table below (in some cases as mean values for individual determinations):

TABLE A

| Example | MEC [μM] |
|---|---|
| 1 | 10 |
| 2 | 1.0 |
| 3 | 3.0 |
| 4 | 3.0 |
| 5 | 3.0 |
| 6 | 10 |
| 7 | 1.0 |
| 8 | 10 |
| 9 | 0.3 |
| 10 | 0.3 |
| 11 | 1.0 |
| 13 | 0.3 |
| 14 | 0.2 |
| 15 | 0.3 |
| 16 | 1.0 |
| 17 | 3.0 |
| 18 | 2.0 |
| 19 | 0.07 |
| 20 | 0.07 |
| 21 | 0.03 |
| 22 | 0.1 |
| 23 | 0.1 |
| 24 | 0.1 |
| 25 | 0.3 |
| 26 | 2.0 |
| 27 | 3.0 |
| 28 | 0.03 |
| 29 | 0.55 |

### B-3. Vasorelaxant Effect In Vitro

Rabbits are stunned by a blow to the neck and exsanguinated. The aorta is removed, freed from adhering tissue and divided into rings of width 1.5 mm, which are placed individually under prestress into 5 ml organ baths with carbogen-sparged Krebs-Henseleit solution at 37° C. having the following composition (each mM): sodium chloride: 119; potassium chloride: 4.8; calcium chloride dihydrate: 1; magnesium sulphate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS-1802 HC, Keithley Instruments Munich), and recorded in parallel on linear recorders. To obtain a contraction, phenylephrine is added to the bath cumulatively in increasing concentration. After several control cycles, the substance to be studied is added in increasing dosage each time in every further run, and the magnitude of the contraction is compared with the magnitude of the contraction attained in the last preceding run. This is used to calculate the concentration needed to reduce the magnitude of the control value by 50% (IC₅₀ value), The standard administration volume is 5 μl; the DMSO content in the bath solution corresponds to 0.1%.

### B-4. Blood Pressure Measurement on Anaesthetized Rats

Male Wistar rats having a body weight of 300-350 g are anaesthetized with thiopental (100 mg/kg i.p.). After tracheotomy, a catheter is introduced into the femoral artery to measure the blood pressure. The substances to be tested are administered as solutions, either orally by means of a gavage or intravenously via the femoral vein (Stasch et al. Br. J. Pharmacol. 2002; 135: 344-355).

### B-5. Radiotelemetry Measurement of Blood Pressure in Conscious, Spontaneously Hypertensive Rats

A commercially available telemetry system from DATA SCIENCES INTERNATIONAL DSI, USA, is employed for the blood pressure measurement on conscious rats described below.

The system consists of 3 main components:

implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The studies are conducted on adult female spontaneously hypertensive rats (SHR Okamoto) with a body weight of >200 g. SHR/NCrl from the Okamoto Kyoto School of Medicine, 1963, were a cross of male Wistar Kyoto rats having greatly elevated blood pressure and female rats having slightly elevated blood pressure, and were handed over at F13 to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6:00 am and at 7:00 pm.

### Transmitter Implantation

The TA11 PA-C40 telemetry transmitters used are surgically implanted under aseptic conditions in the experimental animals at least 14 days before the first experimental use. The animals instrumented in this way can be used repeatedly after the wound has healed and the implant has settled.

For the implantation, the fasted animals are anaesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and the wound is closed layer by layer.

An antibiotic (Tardomyocel COMP, Bayer, 1 ml/kg s.c.) is administered postoperatively for prophylaxis of infection.

### Substances and Solutions

Unless stated otherwise, the substances to be studied are administered orally by gavage to a group of animals in each case (n=6). In accordance with an administration volume of 5 ml/kg of body weight, the test substances are dissolved in suitable solvent mixtures or suspended in 0.5% tylose.

A solvent-treated group of animals is used as control.

### Experimental Outline

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch. They are switched to transmission in the run-up to the experiment. The signals emitted can be detected online by a data acquisition system (Dataquest™ A.R.T. for WINDOWS, DSI) and processed accordingly. The data are stored in each case in a file created for this purpose and bearing the experiment number,

In the standard procedure, the following are measured for 10-second periods in each case:

systolic blood pressure (SBP)
diastolic blood pressure (DBP)
mean arterial pressure (MAP)
heart rate (HR)
activity (ACT).

The acquisition of measurements is repeated under computer control at 5-minute intervals. The source data obtained as absolute values are corrected in the diagram with the currently measured barometric pressure (Ambient Pressure Reference Monitor APR-1) and stored as individual data. Further technical details are given in the extensive documentation from the manufacturer company (DSI).

Unless indicated otherwise, the test substances are administered at 9:00 am on the day of the experiment. Following the administration, the parameters described above are measured over 24 hours.

### Evaluation

After the end of the experiment, the acquired individual data are sorted using the analysis software (DATAQUEST™ A.R.T.™ ANALYSIS). The blank value is assumed here to be the time 2 hours before administration, and so the selected data set encompasses the period from 7:00 am on the day of the experiment to 9:00 am on the following day.

The data are smoothed over a predefinable period by determination of the average (15-minute average) and transfer-red as a text file to a storage medium. The measured values presorted and compressed in this way are transferred to Excel templates and tabulated. For each day of the experiment, the data obtained are stored in a dedicated file bearing the number of the experiment. Results and test protocols are stored in files in paper form sorted by numbers.

### Literature:

Klaus Witte, Kai Hu, Johanna Swiatek, Claudia Müssig, Georg Ertl and Björn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994.

### B-6. Determination of Pharmacokinetic Parameters Following Intravenous and Oral Administration

The pharmacokinetic parameters of the compounds of the invention are determined in male CD-1 mice, male Wistar rats and female beagles. Intravenous administration in the case of mice and rats is effected by means of a species-specific plasma/DMSO formulation, and in the case of dogs by means of a water/PEG400/ethanol formulation. In all species, oral administration of the dissolved substance is performed via gavage, based on a water/PEG400/ethanol formulation. The removal of blood from rats is simplified by inserting a silicone catheter into the right Vena jugularis externa prior to substance administration. The operation is effected at least one day prior to the experiment with isofluran anaesthesia and administration of an analgesic (atropine/rimadyl (3/1) 0.1 ml s.c.). The blood is taken (generally more than 10 time points) within a time window including terminal time points of at least 24 to a maximum of 72 hours after substance administration. The blood is removed into heparinized tubes. The blood plasma is then obtained by centrifugation; if required, it can be stored at −20° C. until further processing.

An internal standard (which may also be a chemically unrelated substance) is added to the samples of the compounds of the invention, calibration samples and qualifiers, and there follows protein precipitation by means of acetonitrile in excess. Addition of a buffer solution matched to the LC conditions, and subsequent vortexing, is followed by centrifugation at 1000 g. The supernatant is analysed by LC-MS/MS using Cₘₐₓ, t_{1/2} reversed-phase columns and variable mobile phase mixtures. The substances are quantified via the peak heights or areas from extracted ion chromatograms of specific selected ion monitoring experiments.

The plasma concentration/time plots determined are used to calculate the pharmacokinetic parameters such as AUC, Cₘₐₓ, t_{1/2} (terminal half-life), F (bioavailability), MRT (mean residence time) and CL (clearance), by means of a validated pharmacokinetic calculation program.

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min. After centrifugation at 1000 g, the plasma concentration is measured (by means of LC-MS/MS; see above) and determined by calculating the ratio of the C_{blood}/Cₚₗₐₛₘₐ value.

### B-7. Metabolic Study

To determine the metabolic profile of the inventive compounds, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with a 1:100 dilution into the incubation mixture. Liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable mobile phase mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

### B-8. Caco-2 Permeability Test

The permeability of a test substance was determined with the aid of the Caco-2 cell line, an established in vitro model for permeability prediction at the gastrointestinal barrier (Artursson, P. and Karlsson, J. (1991). Correlation between oral drug absorption in humans and apparent drug permeability coefficients in human intestinal epithelial (Caco-2) cells. Biochem. Biophys. 175 (3), 880-885). The Caco-2 cells (ACC No. 169, DSMZ, Deutsche Sammiung von Mikroorganismen und Zellkulturen, Braunschweig, Germany) were sown in 24-well plates having an insert and cultivated for 14 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (PₐₚₚB-A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analysed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) was also determined as quality control.

B-9. hERG Potassium Current Assay

The hERG (human ether-a-go-go related gene) potassium current makes a significant contribution to the repolarization of the human cardiac action potential (Scheel et al., 2011). Inhibition of this current by pharmaceuticals can in rare cases cause potentially lethal cardiac arrythmia, and is therefore studied at an early stage during drug development.

The functional hERG assay used here is based on a recombinant HEK293 cell line which stably expresses the KCNH2(HERG) gene (Zhou et al., 1998). These cells are studied by means of the “whole-cell voltage-clamp” technique (Hamill et al., 1981) in an automated system (Patchliner™; Nanion, Munich, Germany), which controls the membrane voltage and measures the hERG potassium current at room temperature. The PatchControlHT™ software (Nanion) controls the Patchliner system, data capture and data analysis. The voltage is controlled by 2 EPC-10 quadro amplifiers controlled by the PatchMasterPro™ software (both: HEKA Elektronik, Lambrecht, Germany). NPC-16 chips with moderate resistance (˜2 MΩ; Nanion) serve as the planar substrate for the voltage clamp experiments.

NPC-16 chips are filled with intra- and extracellular solution (cf. Himmel, 2007) and with cell suspension. After forming a gigaohmn seal and establishing whole-cell mode (including several automated quality control steps), the cell membrane is clamped at the −80 mV holding potential. The subsequent voltage clamp protocol changes the command voltage to +20 mV (for 1000 ms), −120 mV (for 500 ms), and back to the −80 mV holding potential; this is repeated every 12 s. After an initial stabilization phase (about 5-6 minutes), test substance solution is introduced by pipette in rising concentrations (e.g. 0.1, 1, and 10 μmol/l) (exposure about 5-6 minutes per concentration), followed by several washing steps.

The amplitude of the inward “tail” current which is generated by a change in potential from +20 mV to −120 mV serves to quantify the hERG potassium current, and is described as a function of time (IgorPro™ Software). The current amplitude at the end of various time intervals (for example stabilization phase before test substance, first/second/third concentration of test substance) serves to establish a concentration/effect curve, from which the half-maximum inhibiting concentration IC₅₀ of the test substance is calculated.
Hamill O P, Marty A. Neher E, Sakmann B, Sigworth F J. Improved patch-clamp techniques for high-resolution current recording from cells and cell-free membrane patches. Pfluegers Arch 1981; 391:85-100.

Himmel H M. Suitability of commonly used excipients for electrophysiological in-vitro safety pharmacology assessment of effects on hERG potassium current and on rabbit Purkinje fiber action potential. J Pharmacol Toxicol Methods 2007; 56:145-158.
Scheel O, Himmel H, Rascher-Eggstein G, Knott T. Introduction of a modular automated voltage-clamp platform and its correlation with manual human ether-a-go-go related gene voltage-clamp data. Assay Drug Dev Technol 2011; 9:600-607.
Zhou Z F, Gong Q, Ye B, Fan Z, Makielski J C, Robertson G A, January C T. Properties of hERG channels stably expressed in HEK293 cells studied at physiological temperature. Biophys J 1998; 74:230-241.
   B-10. In Vitro Clearance Determinations with Hepatocytes

Incubations with fresh primary hepatocytes were carried out at 37° C. in a total volume of 1.5 ml with a modified Janus® robot (Perkin Elmer) while shaking. The incubations typically contained 1 million living liver cells/ml, approx 1 μM substrate and 0.05 M potassium phosphate buffer (pH=7.4). The final acetonitrile concentration in the incubation was <1%. Aliquots of 125 μl were withdrawn from the incubations after 2, 10, 20, 30, 50, 70 and 90 min and transferred into 96-well filter plates (0.45 μm low-binding hydrophilic PTFE; Millipore: MultiScreen Solvinert). Each of these contained 250 μl of acetonitrile to stop the reaction. After the centrifugation, the filtrates were analysed by MS/MS (typically API 3000).

The in vitro clearances were calculated from the half-lives of the substance degradation, using the following equations:

CL′_{intrinsic}[ml/(min·kg)]=(0.693/in vitro t_{1/2} [min])·(liver weight [g liver/kg body weight])×(cell number [1.1·10̂8]/liver weight [g])/(cell number [1·10̂6]/incubation volume [ml])

CL_{blood} was calculated without taking into account the free fraction (“nonrestricted well stirred model”) by the following equation:

CL_{blood} well-stirred [1/(h·kg)]=(Q_{H}[1/(h·kg)]×CL′_{intrinsic}[1/(h·kg)])/(Q_{H}[1/(h·kg)]+CL′_{intrinsic} [1/(h·kg)])

The species-specific extrapolation factors used for the calculation are summarized in the following table:

| | male/female | | | | | |
|---|---|---|---|---|---|---|
| | | | | Dog | | Man |
| | Mouse m | Mouse f | Rat m/f | m/f | Cyno f | m/f |
| Cell number/g | 110 | 110 | 110 | 110 | 110 | 110 |
| Liver [millions | | | | | | |
| of cells] | | | | | | |
| Liver [g]/ | 50 | 43 | 32 | 39 | 30 | 21 |
| kg Body Weight | | | | | | |
| Liver Blood | 5.4 | 5.4 | 4.2 | 2.1 | 2.5 | 1.3 |
| Flow [1/(h · kg)] | | | | | | |

Fmax values which state the maximum possible bioavailability—based on the hepatic extraction were calculated as follows:

Fₘₐₓ well-stirred [%]=(1−(CL_{blood} well-stirred [1/(h·kg)]/Q_{H}[1/(h·kg)]))×100

### C. WORKING EXAMPLES OF PHARMACEUTICAL COMPOSITIONS

The compounds of the invention can be converted to pharmaceutical formulations as follows:

### Tablet:

### Composition:

100 mg of the compound of the invention, 50 mg of lactose (monohydrate), 50 mg of corn starch (native), 10 ing of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Production:

The mixture of compound of the invention, lactose and starch is granulated with a 5% solution (w/w) of the PVP in water. The granules are dried and then mixed with the magnesium stearate for 5 minutes. This mixture is compressed using a conventional tabletting press (see above for format of the tablet). The guide value used for the pressing is a pressing force of 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol; the compound of the invention is added to the suspension. The water is added while stirring. The mixture is stirred for about 6 h until the swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The resulting solution is subjected to sterile filtration and dispensed into sterile and pyrogen-free injection vessels.

1. Compound of the formula (I) 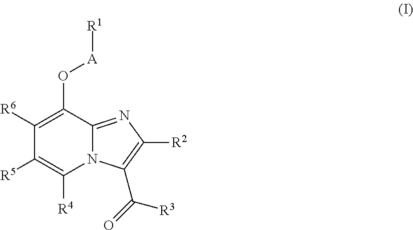
in which
A represents CH₂, CD₂ or CH(CH₃),
R¹ represents (C₃-C₇)-cycloalkyl, pyridyl or phenyl,
where (C₃-C₇)-cycloalkyl may be substituted by 1 to 4 substituents independently of one another selected from the group consisting of fluorine, trifluoromethyl and (C₁-C₄)-alkyl,
and
where phenyl may be substituted by 1 to 4 substituents independently of one another selected from the group consisting of halogen, cyano, monofluoromethyl, difluoromethyl, trifluoromethyl, (C₁-C₄)-alkyl, (C₃-C₅)-cycloalkyl, (C₁-C₄)-alkoxy, difluoromethoxy and trifluoromethoxy,
where pyridyl may be substituted by 1 to 4 substituents independently of one another selected from the group consisting of fluorine, chlorine, monofluoromethyl, difluoromethyl, trifluoromethyl and (C₁-C₄)-alkyl,
R² represents hydrogen, chlorine, (C₁-C₄)-alkyl, (C₁-C₄)-alkoxy, cyclopropyl, cyclobutyl, monofluoromethyl, difluoromethyl or trifluoromethyl,
where (C₁-C₄)-alkyl may be substituted by (C₁-C₄)-alkoxy,
where cyclopropyl and cyclobutyl may be up to disubstituted by fluorine,
R³ represents a group of the formula 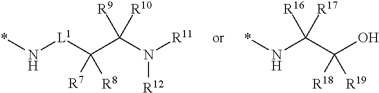
where
* represents the point of attachment to the carbonyl group,
L¹ represents a bond or (C₁-C₄)-alkanediyl,
R⁷ represents hydrogen, fluorine or (C₁-C₆)-alkyl,
in which (C₁-C₆)-alkyl may be substituted up to five times by fluorine,
R⁸ represents hydrogen, fluorine, methyl or ethyl,
R⁹ represents hydrogen or (C₁-C₆)-alkyl,
in which (C₁-C₆)-alkyl may be substituted up to five times by fluorine,
in which (C₁-C₆)-alkyl may be substituted by trimethylsilyl,
R¹⁰ represents hydrogen or (C₁-C₄)-alkyl,
R¹¹ represents hydrogen or (C₁-C₃)-alkyl,
R¹² represents hydrogen or (C₁-C₃)-alkyl,
R¹⁶ represents hydrogen, (C₁-C₆)-alkyl or 5-membered heteroaryl,
in which (C₁-C₆)-alkyl may be substituted up to five times by fluorine,
in which 5-membered heteroaryl is substituted by methyl, difluoromethyl or trifluoromethyl,
R¹⁷ represents hydrogen or methyl,
R¹⁸ represents hydrogen or (C₁-C₄)-alkyl,
in which (C₁-C₆)-alkyl may be substituted up to five times by fluorine,
R¹⁹ represents hydrogen or (C₁-C₄)-alkyl,
R⁴ represents hydrogen,
R⁵ represents hydrogen, chlorine, (C₁-C₄)-alkyl, (C₂-C₄)-alkynyl, (C₃-C₅)-cycloalkyl, difluoromethoxy, trifluoromethoxy or 5- or 6-membered heteroaryl,
where (C₁-C₄)-alkyl may be substituted by (C₁-C₄)-alkoxy,
R⁶ represents hydrogen,
and the N-oxides, salts, solvates, salts of the N-oxides and solvates of the N-oxides and salts thereof.

2. Compound of the formula (I) according to claim 1 in which
A represents CH₂,
R¹ represents pyridyl,
where pyridyl may be substituted by 1 to 3 substituents independently of one another selected from the group consisting of fluorine, difluoromethyl, trifluoromethyl and methyl,
R² represents hydrogen, (C₁-C₄)-alkyl, cyclopropyl, cyclobutyl, monofluoromethyl, difluoromethyl or trifluoromethyl,
where (C₁-C₄)-alkyl may be substituted by (C₁-C₄)-alkoxy,
R³ represents a group of the formula 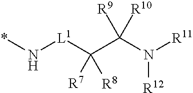
where
* represents the point of attachment to the carbonyl group,
L¹ represents a bond or (C₁-C₄)-alkanediyl,
R⁷ represents hydrogen, fluorine or (C₁-C₆)-alkyl,
in which (C₁-C₆)-alkyl may be substituted up to five times by fluorine,
R⁸ represents hydrogen, fluorine, methyl or ethyl,
R⁹ represents hydrogen or (C₁-C₆)-alkyl,
in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethyl, ethynyl, cyclopropyl, difluoromethoxy, trifluoromethoxy or 5- or 6-membered heteroaryl,
where methyl may be substituted by a methoxy substituent,
R⁶ represents hydrogen,
and the N-oxides, salts, solvates, salts of the N-oxides and solvates of the N-oxides and salts thereof.

3. Compound of the formula (I) according to claim 1 in which
A represents CH₂,
R¹ represents cyclohexyl, pyridyl or phenyl,
where phenyl may be substituted by 1 to 4 substituents independently of one another selected from the group consisting of fluorine, chlorine, bromine and methyl,
and
where pyridyl may be substituted by 1 to 3 substituents independently of one another selected from the group consisting of fluorine, chlorine, bromine and methyl,
R² represents chlorine, (C₁-C₄)-alkyl, methoxy or cyclobutyl,
where (C₁-C₄)-alkyl is substituted by (C₁-C₄)-alkoxy,
where cyclobutyl is disubstituted by fluorine,
R³ represents a group of the formula 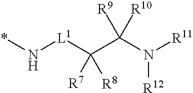
where
* represents the point of attachment to the carbonyl group,
L¹ represents a bond or (C₁-C₄)-alkanediyl,
R⁷ represents hydrogen or fluorine,
R⁸ represents hydrogen or fluorine,
R⁹ represents hydrogen or (C₁-C₆)-alkyl,
in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethyl, ethynyl, cyclopropyl, difluoromethoxy or 5- or 6-membered heteroaryl,
where methyl may be substituted by a methoxy substituent,
R⁶ represents hydrogen,
and the N-oxides, salts, solvates, salts of the N-oxides and solvates of the N-oxides and salts thereof.

4. Compound of the formula (I) according to claim 1 in which
A represents CH₂,
R¹ represents cyclohexyl, pyridyl or phenyl,
where phenyl may be substituted by 1 to 4 substituents independently of one another selected from the group consisting of fluorine, chlorine, bromine and methyl,
where pyridyl may be substituted by 1 to 3 substituents independently of one another selected from the group consisting of fluorine, chlorine, bromine and methyl,
R² represents hydrogen, chlorine, (C₁-C₄)-alkyl, methoxy, cyclopropyl, cyclobutyl, monofluoromethyl, difluoromethyl or trifluoromethyl,
where (C₁-C₄)-alkyl may be substituted by (C₁-C₄)-alkoxy,
R³ represents a group of the formula 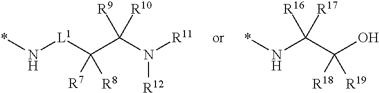
where
* represents the point of attachment to the carbonyl group,
L¹ represents (C₁-C₄)-alkanediyl,
R⁷ represents fluorine,
R⁸ represents fluorine,
R⁹ represents hydrogen or (C₁-C₆)-alkyl,
in which (C₁-C₆)-alkyl may be substituted up to five times by fluorine,
in which (C₁-C₆)-alkyl may be substituted by trimethylsilyl,
R¹⁰ represents hydrogen or (C₁-C₄)-alkyl,
R¹¹ represents hydrogen,
R¹² represents hydrogen,
R¹⁶ represents hydrogen, (C₁-C₆)-alkyl or 5-membered heteroaryl,
in which (C₁-C₆)-alkyl may be substituted up to five times by fluorine, in which 5-membered heteroaryl is substituted by methyl, difluoromethyl or trifluoromethyl,
R¹⁷ represents hydrogen or methyl,
R¹⁸ represents hydrogen or (C₁-C₄)-alkyl,
in which (C₁-C₆)-alkyl may be substituted up to five times by fluorine,
R¹⁹ represents hydrogen or (C₁-C₄)-alkyl,
R⁴ represents hydrogen,
R⁵ represents hydrogen, chlorine, methyl, ethyl, ethynyl, cyclopropyl, difluoromethoxy or 5- or 6-membered heteroaryl,
where methyl may be substituted by a methoxy substituent,
R⁶ represents hydrogen,
and the N-oxides, salts, solvates, salts of the N-oxides and solvates of the N-oxides and salts thereof.

5. Compound of the formula (I) according to claim 1 in which
A represents CH₂,
R¹ represents cyclohexyl, pyridyl or phenyl,
where phenyl may be substituted by 1 to 4 substituents independently of one another selected from the group consisting of fluorine, chlorine, bromine and methyl,
and
where pyridyl may be substituted by 1 to 3 substituents independently of one another selected from the group consisting of fluorine, chlorine, bromine and methyl,
R² represents hydrogen, (C₁-C₄)-alkyl, cyclopropyl, cyclobutyl, monofluoromethyl, difluoromethyl or trifluoromethyl,
where (C₁-C₄)-alkyl may be substituted by (C₁-C₄)-alkoxy,
R³ represents a group of the formula 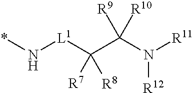
where
* represents the point of attachment to the carbonyl group,
L¹ represents a bond or (C₁-C₄)-alkanediyl,
R⁷ represents hydrogen, fluorine or (C₁-C₆)-alkyl,
in which (C₁-C₆)-alkyl may be substituted up to five times by fluorine,
R⁸ represents hydrogen, fluorine, methyl or ethyl,
R⁹ represents hydrogen or (C₁-C₆)-alkyl,
in which (C₁-C₆)-alkyl may be substituted up to five times by fluorine,
R¹⁰ represents hydrogen or (C₁-C₄)-alkyl,
R¹¹ represents hydrogen,
R¹² represents hydrogen,
R⁴ represents hydrogen,
R⁵ represents hydrogen, chlorine, methyl, ethynyl, cyclopropyl, difluoromethoxy, pyridyl, 1H-pyrazol-1-yl, 1-methyl-1H-pyrazol-4-yl or 1,3-oxazol-5-yl,
where methyl is substituted by methoxy,
R⁶ represents hydrogen,
and the N-oxides, salts, solvates, salts of the N-oxides and solvates of the N-oxides and salts thereof.

6. Process for preparing the compound of the formula (I) as defined in claim 1, comprising:
[A] reacting a compound of the formula (II) 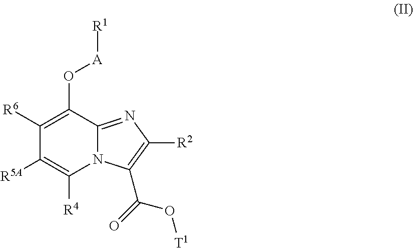
in which A, R¹, R², R⁴ and R⁶ are each as defined in claim 1,
R^{5A} has the meanings given for R⁵ or represents bromine,
and
T¹ represents (C₁-C₄)-alkyl or benzyl,
in an inert solvent in the presence of a suitable base or acid to give a carboxylic acid of the formula (III) 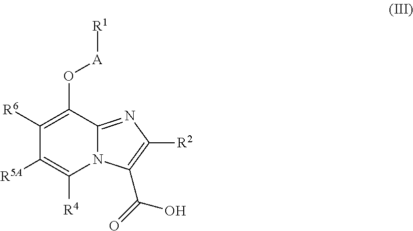
in which A, R¹, R², R⁴ and R⁶ are each as defined above,
and
R^{5A} has the meanings given for R⁵ or represents bromine,
and subsequently reacting the carboxylic acid of the formula III in an inert solvent under amide coupling conditions with an amine of the formula (IV) 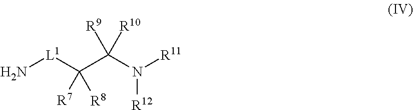
in which L¹, R⁷, R⁸, R⁹, R¹⁰, R¹¹ and R¹² each have the meanings given above,
and, if
R^{5A} represents bromine,
the compounds are reacted in an inert solvent in the presence of a suitable transition metal catalyst, optionally in the presence of a suitable base, with a compound of the formula (IV-A) 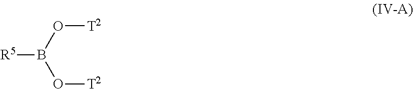
in which
R⁵ has the meaning given above
and
T² represents hydrogen or (C₁-C₄)-alkyl, or the two T² radicals together form a —C(CH₃)₂—C(CH₃)₂— bridge,
or
[B] converting a compound of the formula (III-A) 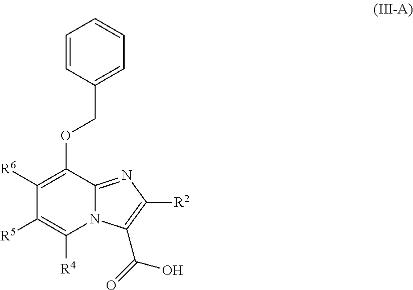
in which R², R⁴, R⁵ and R⁶ each have the meanings given above,
in an inert solvent under amide coupling conditions with an amine of the formula (IV) into a compound of the formula (I-A) 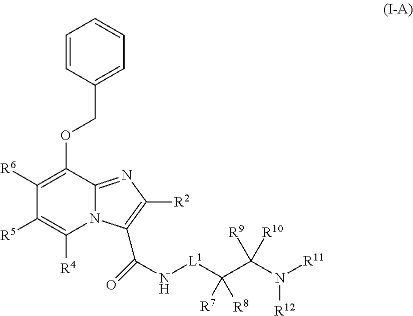
in which L¹, R², R⁴, R⁵, R⁶, R⁷, R⁸, R⁹, R¹⁰, R¹¹ and R¹² each have the meanings given above,
and detaching the benzyl group therefrom by the methods known to the person skilled in the art and reacting the resulting compound of the formula (V) 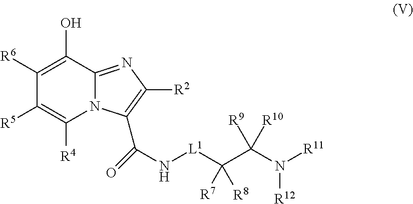
in which L¹, R², R⁴, R⁵, R⁶, R⁷, R⁸, R⁹, R¹⁰, R¹¹ and R¹² each have the meanings given above,
in an inert solvent in the presence of a suitable base with a compound of the formula (VI) 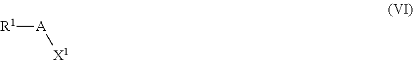
in which A and R¹ have the meaning given above and
X¹ represents a suitable leaving group, in particular chlorine, bromine, iodine, mesylate, triflate or tosylate,
and detaching any protecting groups present, and optionally converting the resulting compounds of the formula (I) with the appropriate (i) solvents and/or (ii) acids or bases to the solvates, salts and/or solvates of the salts thereof.

7. (canceled)

8. (canceled)

9. Medicament comprising the compound of the formula (I) as defined in claim 1 in combination with an inert, non-toxic, pharmaceutically suitable auxiliary.

10. Medicament comprising the compound of the formula (I) as defined in claim 1 in combination with a further active compound selected from the group consisting of organic nitrates, NO donors, cGMP-PDE inhibitors, antithrombotic agents, hypotensive agents and lipid metabolism modifiers.

11. (canceled)

12. Method for treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of at least one compound of the formula (I) as defined in claim 1 to a human or animal in need thereof.

13. Method for treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of the medicament of claim 9 to a human or animal in need thereof.

14. Method for treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of the medicament of claim 10 to a human or animal in need thereof.
